SEQUENCE LISTING (35 sequences)

SEQ ID NO: 1 - DNA, 22 bp, Homo sapiens
  source 1..22: mol_type = unassigned DNA
gcatgactggagcctccatagg

SEQ ID NO: 2 - DNA, 22 bp, Homo sapiens
  source 1..22: mol_type = unassigned DNA
ccagcttccctgacatcccagg

SEQ ID NO: 3 - DNA, 22 bp, Homo sapiens
  source 1..22: mol_type = unassigned DNA
ttcacgtggagcagcaggctgg

SEQ ID NO: 4 - DNA, 22 bp, Homo sapiens
  source 1..22: mol_type = unassigned DNA
cggagcctgagctgtcagatgg

SEQ ID NO: 5 - RNA, 21 nt, synthetic construct
  source 1..21: mol_type = other RNA
  modified_base 20..21: mod_base = OTHER; note = thymine
ggatggaaataaggctaaatt

SEQ ID NO: 6 - RNA, 21 nt, synthetic construct
  source 1..21: mol_type = other RNA
  modified_base 20..21: mod_base = OTHER; note = thymine
ggttcacagatgatacgtttt

SEQ ID NO: 7 - RNA, 21 nt, synthetic construct
  source 1..21: mol_type = other RNA
  modified_base 20..21: mod_base = OTHER; note = thymine
ggaaaatcgtgaagtcgattt

SEQ ID NO: 8 - RNA, 21 nt, synthetic construct
  source 1..21: mol_type = other RNA
tttagccttatttccatccac

SEQ ID NO: 9 - RNA, 21 nt, synthetic construct
  source 1..21: mol_type = other RNA
  modified_base 20: mod_base = OTHER; note = thymine
aacgtatcatctgtgaacctc

SEQ ID NO: 10 - RNA, 21 nt, synthetic construct
  source 1..21: mol_type = other RNA
  modified_base 20..21: mod_base = OTHER; note = thymine
atcgacttcacgattttcctt

SEQ ID NO: 11 - DNA, 22 bp, Homo sapiens
  source 1..22: mol_type = unassigned DNA
taactcccagctattatagagg

SEQ ID NO: 12 - DNA, 22 bp, Homo sapiens
  source 1..22: mol_type = unassigned DNA
gctattatagaggtgcacaggg

SEQ ID NO: 13 - RNA, 21 nt, synthetic construct
  source 1..21: mol_type = other RNA
  modified_base 9: mod_base = OTHER; note = 2&apos;-deoxy 2&apos;-fluoro ribonucleotide
  modified_base 21: mod_base = OTHER; note = inverted abasic nucleotide
  modified_base 20..21: mod_base = OTHER; note = phosphorothiodiester linkage
  modified_base 11..14: mod_base = OTHER; note = 2&apos;-deoxy 2&apos;-fluoro ribonucleotide
tcctatgactgtagattttan

SEQ ID NO: 14 - RNA, 21 nt, synthetic construct
  source 1..21: mol_type = other RNA
  modified_base 1: mod_base = OTHER; note = 2&apos;-deoxy 2&apos;-fluoro ribonucleotide
  modified_base 3: mod_base = OTHER; note = 2&apos;-deoxy 2&apos;-fluoro ribonucleotide
  modified_base 5: mod_base = OTHER; note = 2&apos;-deoxy 2&apos;-fluoro ribonucleotide
  modified_base 7: mod_base = OTHER; note = 2&apos;-deoxy 2&apos;-fluoro ribonucleotide
  modified_base 9: mod_base = OTHER; note = 2&apos;-deoxy 2&apos;-fluoro ribonucleotide
  modified_base 11: mod_base = OTHER; note = 2&apos;-deoxy 2&apos;-fluoro ribonucleotide
  modified_base 13: mod_base = OTHER; note = 2&apos;-deoxy 2&apos;-fluoro ribonucleotide
  modified_base 15: mod_base = OTHER; note = 2&apos;-deoxy 2&apos;-fluoro ribonucleotide
  modified_base 17: mod_base = OTHER; note = 2&apos;-deoxy 2&apos;-fluoro ribonucleotide
  modified_base 19: mod_base = OTHER; note = 2&apos;-deoxy 2&apos;-fluoro ribonucleotide
  modified_base 21: mod_base = OTHER; note = 2&apos;-deoxy 2&apos;-fluoro ribonucleotide
tcctatgactgtagattttat

SEQ ID NO: 15 - RNA, 21 nt, synthetic construct
  source 1..21: mol_type = other RNA
  modified_base 9: mod_base = OTHER; note = 2&apos;-deoxy 2&apos;-fluoro ribonucleotide
  modified_base 1..3: mod_base = OTHER; note = phosphorothiodiester linkage
  modified_base 20..21: mod_base = OTHER; note = phosphorothiodiester linkage
  modified_base 11..14: mod_base = OTHER; note = 2&apos;-deoxy 2&apos;-fluoro ribonucleotide
  modified_base 21: mod_base = OTHER; note = inverted abasic nucleotide
tcctatgactgtagattttan

SEQ ID NO: 16 - RNA, 21 nt, synthetic construct
  source 1..21: mol_type = other RNA
  modified_base 9: mod_base = OTHER; note = 2&apos;-deoxy 2&apos;-fluoro ribonucleotide
  modified_base 1..2: mod_base = OTHER; note = phosphorothiodiester linkage
  modified_base 20..21: mod_base = OTHER; note = phosphorothiodiester linkage
  modified_base 21: mod_base = OTHER; note = inverted abasic nucleotide
  modified_base 11..14: mod_base = OTHER; note = 2&apos;-deoxy 2&apos;-fluoro ribonucleotide
tcctatgactgtagattttan

SEQ ID NO: 17 - RNA, 21 nt, synthetic construct
  source 1..21: mol_type = other RNA
  modified_base 9: mod_base = OTHER; note = 2&apos;-deoxy 2&apos;-fluoro ribonucleotide
  modified_base 20..21: mod_base = OTHER; note = phosphorothiodiester linkage
  modified_base 21: mod_base = OTHER; note = inverted abasic nucleotide
  modified_base 11..14: mod_base = OTHER; note = 2&apos;-deoxy 2&apos;-fluoro ribonucleotide
ttggaaagactgttccaaaan

SEQ ID NO: 18 - RNA, 21 nt, synthetic construct
  source 1..21: mol_type = other RNA
  modified_base 9: mod_base = OTHER; note = 2&apos;-deoxy 2&apos;-fluoro ribonucleotide
  modified_base 20..21: mod_base = OTHER; note = phosphorothiodiester linkage
  modified_base 21: mod_base = OTHER; note = inverted abasic nucleotide
  modified_base 11..14: mod_base = OTHER; note = 2&apos;-deoxy 2&apos;-fluoro ribonucleotide
gggaagaaagatgaagtcgcn

SEQ ID NO: 19 - RNA, 23 nt, synthetic construct
  source 1..23: mol_type = other RNA
  modified_base 7: mod_base = OTHER; note = 2&apos;-deoxy 2&apos;-fluoro ribonucleotide
  modified_base 1..3: mod_base = OTHER; note = phosphorothiodiester linkage
  modified_base 12: mod_base = OTHER; note = 2&apos;-deoxy 2&apos;-fluoro ribonucleotide
  modified_base 21..23: mod_base = OTHER; note = phosphorothiodiester linkage
  modified_base 2: mod_base = OTHER; note = 2&apos;-deoxy 2&apos;-fluoro ribonucleotide
  modified_base 14: mod_base = OTHER; note = 2&apos;-deoxy 2&apos;-fluoro ribonucleotide
ataaaatctacagtcataggatt

SEQ ID NO: 20 - RNA, 23 nt, synthetic construct
  source 1..23: mol_type = other RNA
  modified_base 2: mod_base = OTHER; note = 2&apos;-deoxy 2&apos;-fluoro ribonucleotide
  modified_base 21..23: mod_base = OTHER; note = phosphorothiodiester linkage
  modified_base 4: mod_base = OTHER; note = 2&apos;-deoxy 2&apos;-fluoro ribonucleotide
  modified_base 6: mod_base = OTHER; note = 2&apos;-deoxy 2&apos;-fluoro ribonucleotide
  modified_base 8: mod_base = OTHER; note = 2&apos;-deoxy 2&apos;-fluoro ribonucleotide
  modified_base 10: mod_base = OTHER; note = 2&apos;-deoxy 2&apos;-fluoro ribonucleotide
  modified_base 12: mod_base = OTHER; note = 2&apos;-deoxy 2&apos;-fluoro ribonucleotide
  modified_base 14: mod_base = OTHER; note = 2&apos;-deoxy 2&apos;-fluoro ribonucleotide
  modified_base 16: mod_base = OTHER; note = 2&apos;-deoxy 2&apos;-fluoro ribonucleotide
  modified_base 18: mod_base = OTHER; note = 2&apos;-deoxy 2&apos;-fluoro ribonucleotide
  modified_base 20: mod_base = OTHER; note = 2&apos;-deoxy 2&apos;-fluoro ribonucleotide
  modified_base 22: mod_base = OTHER; note = 2&apos;-deoxy 2&apos;-fluoro ribonucleotide
ataaaatctacagtcataggaat

SEQ ID NO: 21 - RNA, 23 nt, synthetic construct
  source 1..23: mol_type = other RNA
  modified_base 2: mod_base = OTHER; note = 2&apos;-deoxy 2&apos;-fluoro ribonucleotide
  modified_base 1..3: mod_base = OTHER; note = phosphorothiodiester linkage
  modified_base 7: mod_base = OTHER; note = 2&apos;-deoxy 2&apos;-fluoro ribonucleotide
  modified_base 21..23: mod_base = OTHER; note = phosphorothiodiester linkage
  modified_base 12: mod_base = OTHER; note = 2&apos;-deoxy 2&apos;-fluoro ribonucleotide
  modified_base 14: mod_base = OTHER; note = 2&apos;-deoxy 2&apos;-fluoro ribonucleotide
ataaaatctacagtcataggatt

SEQ ID NO: 22 - RNA, 23 nt, synthetic construct
  source 1..23: mol_type = other RNA
  modified_base 12: mod_base = OTHER; note = 2&apos;-deoxy 2&apos;-fluoro ribonucleotide
  modified_base 1..3: mod_base = OTHER; note = phosphorothiodiester linkage
  modified_base 7: mod_base = OTHER; note = 2&apos;-deoxy 2&apos;-fluoro ribonucleotide
  modified_base 21..23: mod_base = OTHER; note = phosphorothiodiester linkage
  modified_base 2: mod_base = OTHER; note = 2&apos;-deoxy 2&apos;-fluoro ribonucleotide
  modified_base 14: mod_base = OTHER; note = 2&apos;-deoxy 2&apos;-fluoro ribonucleotide
ataaaatctacagtcataggatt

SEQ ID NO: 23 - RNA, 23 nt, synthetic construct
  source 1..23: mol_type = other RNA
  modified_base 12: mod_base = OTHER; note = 2&apos;-deoxy 2&apos;-fluoro ribonucleotide
  modified_base 7: mod_base = OTHER; note = 2&apos;-deoxy 2&apos;-fluoro ribonucleotide
  modified_base 1..3: mod_base = OTHER; note = phosphorothiodiester linkage
  modified_base 2: mod_base = OTHER; note = 2&apos;-deoxy 2&apos;-fluoro ribonucleotide
  modified_base 21..23: mod_base = OTHER; note = phosphorothiodiester linkage
  modified_base 14: mod_base = OTHER; note = 2&apos;-deoxy 2&apos;-fluoro ribonucleotide
tttttggaacagtctttccaatt

SEQ ID NO: 24 - RNA, 23 nt, synthetic construct
  source 1..23: mol_type = other RNA
  modified_base 2: mod_base = OTHER; note = 2&apos;-deoxy 2&apos;-fluoro ribonucleotide
  modified_base 7: mod_base = OTHER; note = 2&apos;-deoxy 2&apos;-fluoro ribonucleotide
  modified_base 1..3: mod_base = OTHER; note = phosphorothiodiester linkage
  modified_base 21..23: mod_base = OTHER; note = phosphorothiodiester linkage
  modified_base 12: mod_base = OTHER; note = 2&apos;-deoxy 2&apos;-fluoro ribonucleotide
  modified_base 14: mod_base = OTHER; note = 2&apos;-deoxy 2&apos;-fluoro ribonucleotide
agcgacttcatctttcttccctt

SEQ ID NO: 25 - RNA, 19 nt, synthetic construct
  source 1..19: mol_type = other RNA
tttagccttatttccatcc

SEQ ID NO: 26 - RNA, 19 nt, synthetic construct
  source 1..19: mol_type = other RNA
aacgtatcatctgtgaacc

SEQ ID NO: 27 - RNA, 19 nt, synthetic construct
  source 1..19: mol_type = other RNA
atcgacttcacgattttcc

SEQ ID NO: 28 - RNA, 22 nt, synthetic construct
  source 1..22: mol_type = other RNA
cctctataatagctgggagtta

SEQ ID NO: 29 - RNA, 22 nt, synthetic construct
  source 1..22: mol_type = other RNA
ccctgtgcacctctataatagc

SEQ ID NO: 30 - DNA, 19 bp, synthetic construct
  source 1..19: mol_type = other DNA
aaaatctacagtcatagga

SEQ ID NO: 31 - DNA, 19 bp, synthetic construct
  source 1..19: mol_type = other DNA
ataaaatctacagtcatag

SEQ ID NO: 32 - DNA, 19 bp, synthetic construct
  source 1..19: mol_type = other DNA
gggcatatctggaagccat

SEQ ID NO: 33 - DNA, 19 bp, synthetic construct
  source 1..19: mol_type = other DNA
  modified_base 4..6: mod_base = OTHER; note = Beta-D-Methyleneoxy nucleotide
  modified_base 17..19: mod_base = OTHER; note = Beta-D-Methyleneoxy nucleotide
  modified_base 6: mod_base = m5c
  modified_base 4..19: mod_base = OTHER; note = phosphorothiodiester linkage
aaaatctacagtcatagga

SEQ ID NO: 34 - DNA, 19 bp, synthetic construct
  source 1..19: mol_type = other DNA
  modified_base 17..19: mod_base = OTHER; note = Beta-D-Methyleneoxy nucleotide
  modified_base 4..6: mod_base = OTHER; note = Beta-D-Methyleneoxy nucleotide
  modified_base 4..19: mod_base = OTHER; note = phosphorothiodiester linkage
ataaaatctacagtcatag

SEQ ID NO: 35 - DNA, 19 bp, synthetic construct
  source 1..19: mol_type = other DNA
  modified_base 17..19: mod_base = OTHER; note = Beta-D-Methyleneoxy nucleotide
  modified_base 4..6: mod_base = OTHER; note = Beta-D-Methyleneoxy nucleotide
  modified_base 4: mod_base = m5c
  modified_base 4..19: mod_base = OTHER; note = phosphorothiodiester linkage
  modified_base 17: mod_base = m5c
gggcatatctggaagccat

(19) United States
(12) Patent Application Publication
(10) Pub. No.: US 2025/0011779 A1
(43) Pub. Date: Jan. 9, 2025

(54) COMPOSITIONS AND METHODS FOR ENHANCING GENE SILENCING ACTIVITY OF OLIGONUCLEOTIDE COMPOUNDS

(71) Applicant: AMGEN INC., Thousand Oaks, CA (US)

(72) Inventors: Jiamiao LU, Mountain View, CA (US); Michael OLLMANN, San Carlos, CA (US); Patrick COLLINS, Millbrae, CA (US); Chi-Ming LI, Thousand Oaks, CA (US); Songli WANG, Palo Alto, CA (US)

(73) Assignee: AMGEN INC., Thousand Oaks, CA (US)

(21) Appl. No.: 18/697,901

(22) PCT Filed: Oct. 4, 2022
(86) PCT No.: PCT/US2022/045664

§ 371 Date: Apr. 2, 2024

Related U.S. Application Data
(60) Provisional application No. 63/252,596, filed on Oct. 5, 2021.

(51) Int. Cl. C12N 15/113

(52) U.S. Cl. CPC C12N 15/113; C12N 15/1137; C12Y 306/05002; C12N 2310/11; C12N 2310/14; C12N 2310/315; C12N 2310/321; C12N 2310/322; C12N 2310/3231; C12N 2310/351

(57) ABSTRACT

The present invention relates to compositions and methods for enhancing the gene silencing activity of oligonucleotide compounds. In particular, the invention relates to inhibiting the expression or activity of suppressor proteins, such as RAB18, ZW10, STX18, SCFD2, NAPG, SAMD4B, or VPS37A, to increase the efficacy of ligand-conjugated oligonucleotide compounds in reducing the expression of target genes in a cell.

### CROSS-REFERENCE TO RELATED APPLICATIONS

This application claims the benefit of U.S. Provisional Application No. 63/252,596, filed Oct. 5, 2021, which is hereby incorporated by reference in its entirety.

### DESCRIPTION OF THE TEXT FILE SUBMITTED ELECTRONICALLY

The present application contains a Sequence Listing, which has been submitted electronically in XML format and is hereby incorporated by reference in its entirety. The computer readable format copy of the Sequence Listing, which was created on Oct. 3, 2022, is named A-2846-WO01-SEC_ST26.xml and is 81.2 kilobytes in size.

### FIELD OF THE INVENTION

The present invention relates to the identification of proteins that act to suppress the gene silencing activity of oligonucleotide compounds in a cell. More specifically, the present invention relates to compositions and methods for enhancing the efficacy of ligand-conjugated oligonucleotide compounds in reducing the expression of target genes in a cell by inhibiting the expression or activity of such suppressor proteins, such as RAB18, ZW10, STX18, SCFD2, NAPG, SAMD4B, or VPS37A. The described methods are particularly useful for increasing the potency of ligand-conjugated oligonucleotide compounds administered for therapeutic purposes.

### BACKGROUND OF THE INVENTION

Nucleic acid-based therapeutics, such as small interfering RNA (siRNA) molecules and antisense oligonucleotides, have developed rapidly in recent years, despite the challenges associated with delivery of large, highly charged nucleic acids. Compared with traditional drug molecules, siRNA molecules and antisense oligonucleotides are highly potent and capable of acting on previously “non-druggable” targets (Juliano, Nucleic Acids Res, Vol. 44:6518-6548, 2016; Dowdy, Nat Biotechnol, Vol. 35:222-229, 2017; and Khvorova and Watts, Nat Biotechnol, Vol. 35:238-248, 2017). More impressively, the duration of siRNA-mediated gene silencing in particular has been shown to last for several months (Nair et al., Nucleic Acids Res, Vol. 45:10969-10977, 2017; Juliano, 2016; Dowdy, 2017; and Khvorova and Watts, 2017, supra).

Delivery of oligonucleotide therapeutic molecules to the liver has been established, with conjugation of the oligonucleotide to ligands comprising N-acetylgalactosamine (GalNAc), which binds to the asialoglycoprotein receptor (ASGPR) that is highly expressed on the surface of hepatocytes (see, e.g., Nair et al., J Am Chem Soc, Vol. 136:16958-16961, 2014). The siRNA molecule is then delivered across the plasma membrane by a receptor-mediated endocytosis mechanism into endosomes (see, e.g., Baenziger and Fiete, Cell, Vol. 22:611-620, 1980; Prakash et al., Nucleic Acids Res, Vol. 42:8796-8807, 2014). As endosomes mature, the internal pH drops and causes GalNAc-conjugated oligonucleotides to be released from ASGPRs, which are then quickly recycled back to the cell surface, while the GalNAc-conjugated oligonucleotides remain inside the endosome (Prakash et al., 2014, supra). To access the target mRNA to effectively inhibit protein expression, the oligonucleotides must escape the endosome into the cytosol and engage the RNA induced silencing complex (RISC), in the case of siRNA molecules, or RNase H, in the case of antisense oligonucleotides. Less than 1% of the oligonucleotide molecules within the endosome are capable of escape into the cytosol (Gilleron et al., Nat Biotechnol, Vol. 31:638-646, 2013). The intracellular trafficking and escape steps for therapeutic oligonucleotide molecules are very inefficient, and the underlying mechanisms are not fully understood (Springer and Dowdy, Nucleic Acid Ther, Vol. 28:109-118, 2018; Prakash et al., 2014, supra).

Thus, there remains a need in the art to improve the intracellular delivery of therapeutic oligonucleotides to their site of action in the cytosol, which in turn may improve the potency of these molecules.

### SUMMARY OF THE INVENTION

The present invention is based, in part, on the identification of cellular proteins that act to inhibit or suppress the gene silencing activity of oligonucleotide compounds, particularly ligand-conjugated oligonucleotide compounds. Accordingly, the methods of the invention described herein provide means for enhancing the gene silencing activity of ligand-conjugated oligonucleotide compounds, particularly therapeutic oligonucleotide compounds, by inhibiting the expression or activity of such suppressor proteins in a target cell or subject.

In some embodiments, the methods comprise inhibiting the expression or activity of a suppressor protein in a cell, for example by contacting the cell with an inhibitor of the suppressor protein, and contacting the cell with an oligonucleotide compound comprising a sequence that is substantially or fully complementary to a target gene sequence (e.g. a target gene-directed oligonucleotide compound), wherein the oligonucleotide compound is covalently attached to a ligand of a receptor expressed on the surface of the cell. The cell may be in vitro or in vivo. In some embodiments, the cell is in a subject in need of reduced expression of the target gene. Thus, in certain embodiments, the present invention also includes methods for reducing expression of a target gene in a subject comprising administering to the subject an inhibitor of a suppressor protein and an oligonucleotide compound comprising a sequence that is substantially or fully complementary to a sequence of the target gene (e.g. a target gene-directed oligonucleotide compound), wherein the oligonucleotide compound is covalently attached to a ligand. In some embodiments, the target gene is a human gene and may be a gene expressed in liver cells or tissue. In these and other embodiments, the expression of the target gene is associated with a disease or disorder in the subject and therefore, the oligonucleotide compound may be therapeutic.

The target gene-directed oligonucleotide compounds used in the methods of the invention can be single-stranded or double-stranded. For instance, in some embodiments, the oligonucleotide compounds are single-stranded antisense oligonucleotides comprising a sequence that is substantially or fully complementary to a target gene sequence. In such embodiments, the antisense oligonucleotides may be about 15 to about 30 nucleotides in length. In other embodiments, the oligonucleotide compounds are siRNA molecules comprising a sense strand and an antisense strand, wherein the antisense strand comprises a sequence that is substantially or fully complementary to a sequence of the target gene. In some embodiments, the sense strand may comprise a sequence that is sufficiently complementary to the sequence of the antisense strand to form a duplex region of about 15 to about 30 base pairs in length. In these and other embodiments, the sense and antisense strands are each independently about 19 to about 30 nucleotides in length.

The target gene-directed oligonucleotide compounds used in the methods of the invention may comprise one or more modified nucleotides, including nucleotides having modifications to the ribose ring, nucleobase, or phosphodiester backbone. In some embodiments, the oligonucleotide compounds comprise one or more 2′-modified nucleotides. Such 2′-modified nucleotides can include 2′-fluoro modified nucleotides, 2′-O-methyl modified nucleotides, 2′-O-methoxyethyl modified nucleotides, 2′-O-alkyl modified nucleotides, 2′-O-allyl modified nucleotides, bicyclic nucleic acids (BNA), deoxyribonucleotides, or combinations thereof. In some embodiments, all of the nucleotides in the oligonucleotide compounds are modified nucleotides. In certain embodiments, the target gene-directed oligonucleotide compounds used in the methods of the invention comprise at least one backbone modification, such as a modified internucleotide or internucleoside linkage. For instance, in some embodiments, the oligonucleotide compounds comprise one or more phosphorothioate internucleotide linkages.

In certain embodiments of the methods of the invention, the target gene-directed oligonucleotide compounds are covalently attached to a ligand of a receptor expressed in the cell or tissue into which the oligonucleotide compounds are intended to be delivered. In some embodiments, the ligand comprises a cholesterol moiety, a vitamin, a steroid, a bile acid, a folate moiety, a fatty acid, a carbohydrate, a glycoside, or antibody or antigen-binding fragment thereof. In particular embodiments, the ligand targets delivery of the oligonucleotide compounds to liver cells (e.g. hepatocytes). In these and other embodiments, the ligand may be a ligand of the asialoglycoprotein receptor and comprise galactose, galactosamine, or N-acetyl-galactosamine (GalNAc). In certain embodiments, the ligand comprises a multivalent galactose or multivalent GalNAc moiety, such as a trivalent or tetravalent galactose or GalNAc moiety. The ligand may be covalently attached to the 5′ or 3′ end of an oligonucleotide of the oligonucleotide compound, optionally through a linker.

Inhibitors of a suppressor protein can be any type of molecule or agent that reduces the expression or activity of the suppressor protein in the cell to which the target gene-directed oligonucleotide compound will be delivered. In some embodiments of the methods of the invention, the suppressor protein is RAB18, ZW10, STX18, SCFD2, NAPG, SAMD4B, VPS37A, YAP1, CCNE1, SLC30A9, TEDC1, HIF1AN, or TRAF2. In certain embodiments of the methods of the invention, the suppressor protein is RAB18, ZW10, STX18, SCFD2, NAPG, SAMD4B, or VPS37A. In certain other embodiments of the methods of the invention, the suppressor protein is RAB18, ZW10, or STX18. In one particular embodiment, the suppressor protein is RAB18.

In some embodiments of the methods of the invention, inhibitors of the suppressor protein, such as any of the suppressor proteins described herein, may be oligonucleotide-based inhibitors that reduce the expression of nucleic acids (e.g. mRNA) encoding the suppressor protein. For instance, in some embodiments, the inhibitor of the suppressor protein is an oligonucleotide compound as described herein, wherein the oligonucleotide compound comprises a sequence that is substantially or fully complementary to an mRNA sequence encoding the suppressor protein (e.g. suppressor protein-directed oligonucleotide compound). Such suppressor protein-directed oligonucleotide compounds may be single-stranded, for example, be a single-stranded antisense oligonucleotide comprising a sequence that is substantially or fully complementary to an mRNA sequence encoding the suppressor protein. In alternative embodiments, the suppressor protein-directed oligonucleotide compounds may be double-stranded and comprise, for example, an siRNA or shRNA. In some embodiments of the methods of the invention, the suppressor protein-directed oligonucleotide compounds are siRNA molecules comprising a sense strand and antisense strand, wherein the antisense strand comprises a sequence that is substantially or fully complementary to an mRNA sequence encoding the suppressor protein. The suppressor protein-directed oligonucleotide compounds may comprise one or more modified nucleotides (e.g. 2′-modified nucleotides) or modified internucleotide or internucleoside linkages (e.g. phosphorothioate internucleotide linkages) as described herein. In some embodiments, the suppressor protein-directed oligonucleotide compounds may be covalently attached to any of the ligands described herein. In one such embodiment, the ligand covalently attached to the suppressor protein-directed oligonucleotide compound may be the same as the ligand covalently attached to the target gene-directed oligonucleotide compound. In another embodiment, the ligand covalently attached to the suppressor protein-directed oligonucleotide compound may be different than the ligand covalently attached to the target gene-directed oligonucleotide compound, but both ligands are ligands of a receptor(s) expressed in the same cell type or tissue.

In other embodiments of the methods of the invention, inhibitors of the suppressor protein, such as any of the suppressor proteins described herein, are gene modifying agents that modify a gene encoding the suppressor protein to encode a variant of the suppressor protein that has reduced activity or function or to eliminate expression of the gene entirely (i.e. knockout out the gene). In certain embodiments, the gene modifying agent comprises a transcription activator-like effector nuclease (TALEN) or a zinc finger nuclease (ZFN) or a vector/polynucleotide encoding the nuclease. In certain other embodiments, the gene modifying agent comprises: (i) a Cas nuclease or vector/polynucleotide encoding the nuclease and (ii) a guide RNA or vector/polynucleotide comprising a guide RNA expression cassette, wherein the guide RNA comprises a sequence that is complementary to a portion of the gene sequence encoding the suppressor protein. Vectors encoding the nuclease and/or guide RNA expression cassette can be viral vectors, such as lentiviral vectors.

### BRIEF DESCRIPTION OF THE DRAWINGS

FIG. 1A depicts bar graphs of SLC3A2 expression in a Hep3B parental cell line or one of three different stable Hep3BCas9 cell lines following transduction with one of two gRNA lentiviral vectors, SLC3A2-83 or SLC3A2-84.

FIG. 1B depicts bar graphs of ASGR1 expression in a Hep3B parental cell line or one of three different stable Hep3BCas9 cell lines following transduction with one of two gRNA lentiviral vectors, ASGR1-77 or ASGR1-78.

FIGS. 2A and 2B are bar graphs of viable cell counts measured on day 3 (FIG. 2A) and day 6 (FIG. 2B) post-treatment with 100 μM 6-thioguanine (6TG) in one of four different treatment groups. Hep3BCas9 cells transduced with a gRNA lentivirus library were treated with a GalNAc moiety-conjugated HPRT1 siRNA alone (HPRT1-si), the GalNAc moiety-conjugated HPRT1 siRNA with 6TG (HPRT1-si+6-TG), 6-TG alone (6-TG), or neither the siRNA nor 6TG (negative control). The viable cell count measured by ViCell on day 3 and day 6 post-6TG treatment for each treatment group was normalized by negative control group readings. The resulting normalized viability percentage of each group at both time points is shown as average±standard deviation.

FIG. 3A is a scatterplot depicting enriched genes in both 150 nM siRNA+6TG treated samples (150si6TGd9) vs. no siRNA but 6TG treated samples (nosi6TGd9) and 750 nM siRNA+6TG treated samples (750si6TGd9) vs. no siRNA but 6TG treated samples (nosi6TGd9). A total of 17 genes were identified with a false discovery rate (FDR)<0.2 (labeled black solid dots).

FIG. 3B is a scatterplot depicting enriched genes from 750 nM siRNA+6TG treated samples (750si6TGd9) vs. no siRNA but 6TG treated samples (nosi6TGd9) with depleted genes in 6TG only vs no siRNA no 6TG samples. The horizontal axis indicates the sensitivity to 6TG. The dashed box outlines 8 genes with FDR<0.2 that were heavily depleted upon 6TG treatment.

FIG. 4A is a line graph showing the percentage of RAB18 mRNA levels in Hep3B cells treated for 24 hours with various concentrations of one of three different RAB18-targeted siRNA molecules. RNA samples were extracted from Hep3B cells treated with three different siRNA molecules targeting RAB18 gene at various concentrations at 24 hr post treatment. The cDNA samples synthesized from RNA through reverse transcription were then subject to ddPCR analysis. The RAB18 ddPCR readings were normalized to those for housekeeping TBP gene to calculate the percentage of RAB18 mRNA level.

FIG. 4B is a bar graph of RAB18 mRNA levels in Hep3B cells transfected with a non-targeting control siRNA molecule (siNTC) or a RAB18-targeted siRNA molecule (siRAB18) four days post treatment with a GalNAc moiety-conjugated HPRT1 siRNA molecule. Hep3B cells were pretreated with siRAB18-3 or siNTC molecules through transfection. 24 hr later, after washing off the transfection media, the cells were treated with a GalNAc moiety-conjugated HPRT1 siRNA molecule (duplex no. 8172) at various concentrations. On day 4 post treatment with duplex no. 8172, the cells were harvested for ddPCR measurement of RAB18 mRNA level.

FIG. 4C shows dose response curves for a GalNAc moiety-conjugated HPRT1 siRNA molecule (duplex no. 8172) in Hep3B cells transfected with either a non-targeting control siRNA molecule (siNTC) or a RAB18-targeted siRNA molecule (siRAB18). On day 4 post treatment with duplex no. 8172, HPRT1 mRNA levels were measured by ddPCR from the cells harvested from the experiment described in FIG. 4B. HPRT1 mRNA levels are expressed as a percentage normalized by housekeeping TBP gene readings and no siRNA (PBS only) treated control group.

FIG. 5 is a graph of percent cell lysis as a function of concentration of the indicated GalNAc moiety-conjugated siRNA molecules. Hep3BCas9 cells and two different RAB18 knockout cell pools (RAB18 KO_1 and RAB18 KO_2) were treated for 3 days with various concentrations of either a GalNAc-HPRT1 siRNA conjugate molecule (HPRT1-si) or a GalNAc-PPIB siRNA conjugate molecule (PPIB-si). Cells were then subject to a live/dead selection screen using 100 μM 6TG. Viable cells were detected using CellTiter-Glo reagents on day 6 post-6TG treatment.

FIG. 6A shows dose response curves for a GalNAc moiety-conjugated HPRT1 siRNA molecule (duplex no. 8172) in Hep3BCas9 cells and two different RAB18 knockout cell pools (RAB18 KO_1 and RAB18 KO_2). Cells were treated for 4 days with various concentrations of either a GalNAc-HPRT1 siRNA conjugate molecule (HPRT1-si) or a GalNAc-PPIB siRNA conjugate molecule (PPIB-si) as a control. mRNA levels were measured by ddPCR. HPRT1 mRNA levels are expressed as a percentage normalized by housekeeping TBP gene readings and no siRNA (PBS only) treated control group.

FIG. 6B shows dose response curves for a GalNAc moiety-conjugated ASGR1 siRNA molecule (duplex no. 16084) in Hep3BCas9 cells and two different RAB18 knockout cell pools (RAB18 KO_1 and RAB18 KO_2). Cells were treated for 4 days with various concentrations of either a GalNAc-ASGR1 siRNA conjugate molecule (ASGR1-si) or a GalNAc-PPIB siRNA conjugate molecule (PPIB-si) as a control. mRNA levels were measured by ddPCR. ASGR1 mRNA levels are expressed as a percentage normalized by housekeeping TBP gene readings and no siRNA (PBS only) treated control group.

FIG. 6C shows dose response curves for a GalNAc moiety-conjugated PPIB siRNA molecule (duplex no. 8714) in Hep3BCas9 cells and two different RAB18 knockout cell pools (RAB18 KO_1 and RAB18 KO_2). Cells were treated for 4 days with various concentrations of either a GalNAc-PPIB siRNA conjugate molecule (PPIB-si) or a GalNAc-HPRT1 siRNA conjugate molecule (HPRT1-si) as a control. mRNA levels were measured by ddPCR. PPIB mRNA levels are expressed as a percentage normalized by housekeeping TBP gene readings and no siRNA (PBS only) treated control group.

FIG. 7 depicts dose response curves for a GalNAc moiety-conjugated HPRT1 siRNA molecule (duplex no. 8172) in Hep3BCas9 cells and two different RAB18 knockout cell pools (RAB18 KO_1 and RAB18 KO_2). Cells were pre-treated with an anti-ASGR1 antibody (7E11), an isotype control antibody (isotype), or no antibody for 30 minutes. The GalNAc-HPRT1 siRNA conjugate was then added to the cells at various concentrations. mRNA levels were measured by ddPCR on day 4 post-siRNA treatment. HPRT1 mRNA levels are expressed as a percentage normalized by housekeeping TBP gene readings and no siRNA (PBS only) treated control group.

FIG. 8 depicts dose response curves for an unconjugated HPRT1 siRNA molecule (duplex no. 17629) with and without lipofectamine reagent (RNAiMAX) in Hep3BCas9 cells and RAB18 knockout cells (RAB18 KO). Cells were treated for 4 days with various concentrations of the unconjugated HPRT1 siRNA molecule (HPRT1-si 17629) alone or with lipofectamine RNAiMAX reagent. mRNA levels were measured by ddPCR. HPRT1 mRNA levels are expressed as a percentage normalized by housekeeping TBP gene readings and no siRNA (PBS only) treated control group.

FIG. 9 shows dose response curves for GalNAc moiety-conjugated HPRT1 single-stranded antisense oligonucleotide (ASO) molecules (compound nos. 15469 and 15470) and a control GalNAc moiety-conjugated PNPLA3 ASO molecule (compound no. 15472) in Hep3BCas9 cells and RAB18 knockout cells (RAB18 KO). Cells were treated for 4 days with various concentrations of the different GalNAc-ASO conjugate molecules. mRNA levels were measured by ddPCR. HPRT1 mRNA levels are expressed as a percentage normalized by housekeeping TBP gene readings and no ASO (PBS only) treated control group.

### DETAILED DESCRIPTION

The present invention is based, in part, on the identification of intracellular proteins that negatively impact the gene silencing activity of oligonucleotide compounds, such as siRNA molecules. As described further herein, suppression or inhibition of the expression or activity of such suppressor proteins significantly increases the gene silencing activity of the oligonucleotide compounds, thereby potentially expanding the therapeutic utility of the oligonucleotide compounds. The methods of the invention are particularly useful for enhancing or increasing the gene silencing activity of ligand-conjugated oligonucleotide compounds, which enter a cell through a receptor-mediated endocytosis pathway, as some of the identified suppressor proteins, such as RAB18, are believed to play a role in intracellular trafficking of endosomes. Accordingly, in certain embodiments, the present invention provides methods for enhancing the silencing activity of an oligonucleotide compound in a cell comprising inhibiting the expression or activity of a suppressor protein in the cell and contacting the cell with the oligonucleotide compound, wherein the oligonucleotide compound comprises a sequence that is substantially complementary to the sequence of a target gene.

As used herein, an “oligonucleotide compound” is a compound comprising at least one oligonucleotide that has a nucleotide sequence that is sufficiently complementary to a target nucleic acid sequence to hybridize to the target nucleic acid and cause gene silencing activity. “Hybridize” or “hybridization” refers to the pairing of complementary oligonucleotides, typically via hydrogen bonding (e.g. Watson-Crick, Hoogsteen or reversed Hoogsteen hydrogen bonding) between complementary bases in the two oligonucleotides. As used herein, a first sequence is “complementary” to a second sequence if an oligonucleotide comprising the first sequence can hybridize to an oligonucleotide comprising the second sequence to form a duplex region under certain conditions, such as physiological conditions. Other such conditions can include moderate or stringent hybridization conditions, which are known to those of skill in the art. A first sequence is considered to be fully complementary (100% complementary) to a second sequence if an oligonucleotide comprising the first sequence base pairs with an oligonucleotide comprising the second sequence over the entire length of one or both nucleotide sequences without any mismatches. A sequence is “substantially complementary” to a target sequence if the sequence is at least about 80%, 85%, 90%, 95%, 96%, 97%, 98%, or 99% complementary to a target sequence. Percent complementarity can be calculated by dividing the number of bases in a first sequence that are complementary to bases at corresponding positions in a second or target sequence by the total length of the first sequence. A sequence may also be said to be substantially complementary to another sequence if there are no more than 5, 4, 3, or 2 mismatches over a 30 base pair duplex region when the two sequences are hybridized.

The oligonucleotide compounds used in the methods of the invention comprise at least one oligonucleotide that has a region having a sequence that is substantially or fully complementary to a target gene sequence. A target gene sequence generally refers to a nucleic acid sequence that comprises a partial or complete coding sequence for a polypeptide. The target gene sequence may also include a non-coding region, such as the 5′ or 3′ untranslated region (UTR) or promoter region. In certain embodiments, the target gene sequence is a messenger RNA (mRNA) sequence. An mRNA sequence refers to any messenger RNA sequence, including splice variants, encoding a protein, protein variants, or isoforms from any species (e.g. mouse, rat, non-human primate, human). In one embodiment, the target gene sequence is an mRNA sequence encoding a human protein. A target gene sequence can also be an RNA sequence other than an mRNA sequence, such as a tRNA sequence, microRNA sequence, or viral RNA sequence.

In certain embodiments of the methods of the invention, the oligonucleotide compound comprises at least one oligonucleotide that has a region that is substantially complementary or fully complementary to at least 10 consecutive nucleotides of a target gene sequence. In some embodiments, the region of the target gene sequence to which the oligonucleotide comprises a region of complementarity can range from about 10 to about 30 consecutive nucleotides, from about 15 to about 30 consecutive nucleotides, from about 16 to about 28 consecutive nucleotides, from about 18 to about 26 consecutive nucleotides, from about 17 to about 24 consecutive nucleotides, from about 15 to about 20 consecutive nucleotides, from about 19 to about 30 consecutive nucleotides, from about 19 to about 25 consecutive nucleotides, from about 19 to about 23 consecutive nucleotides, or from about 19 to about 21 consecutive nucleotides.

“Gene silencing activity” or “silencing activity” refers to downregulation or reduction of expression of a target gene at the level of transcription or translation. Gene silencing activity encompasses reduction of gene expression via an RNA interference mechanism, RNase H-mediated degradation, and steric inhibition. RNA interference is the process by which a nucleic acid molecule induces the cleavage and degradation of a target RNA molecule (e.g. messenger RNA or mRNA molecule) in a sequence-specific manner, e.g. through an RNA-induced silencing complex (RISC) pathway. RNase H-mediated degradation results when an oligonucleotide comprising a stretch or gap of deoxyribonucleotides hybridizes to a target RNA molecule (e.g. mRNA molecule) creating a DNA/RNA hybrid, which is a substrate for the ribonuclease RNase H, thereby causing the cleavage of the target RNA molecule by RNase H. Gene silencing activity can also occur through steric inhibition where an oligonucleotide hybridizes to a target nucleic acid sequence and prevents transcription by RNA polymerase (e.g. when the target nucleic acid sequence is a promoter region) or prevents translation by the ribosome when the target nucleic acid sequence is an mRNA molecule.

In some embodiments of the methods of the invention, the oligonucleotide compound is single-stranded. For example, the oligonucleotide compound comprises or consists of a single oligonucleotide that does not comprise any duplex regions or self-complementary regions. In certain embodiments, the oligonucleotide compound is a single-stranded antisense oligonucleotide comprising a sequence that is substantially complementary or fully complementary to the sequence of a target gene. The single-stranded antisense oligonucleotide may be from about 10 to about 30 nucleotides in length, from about 15 to about 30 nucleotides in length, from about 12 to about 28 nucleotides in length, from about 18 to about 26 nucleotides in length, from about 20 to about 30 nucleotides in length, from about 15 to about 20 nucleotides in length, form about 19 to about 25 nucleotides in length, from about 19 to about 23 nucleotides in length, from about 19 to about 21 nucleotides in length, from about 21 to about 25 nucleotides in length, or from about 20 to about 23 nucleotides in length. In some embodiments, the single-stranded antisense oligonucleotide is about 18, about 19, about 20, about 21, about 22, about 23, about 24, or about 25 nucleotides in length.

In other embodiments of the methods of the invention, the oligonucleotide compound is double-stranded. In some such embodiments, the oligonucleotide compound comprises or consists of two antiparallel oligonucleotides that are sufficiently complementary to each other to hybridize to form a duplex region. The oligonucleotide comprising a region having a sequence that is substantially complementary or fully complementary to a target gene sequence (e.g. target mRNA) is referred to as the “antisense strand” or “guide strand.” The “sense strand” or “passenger strand” refers to the oligonucleotide that includes a region that is substantially complementary or fully complementary to a region of the antisense strand. In some embodiments, the sense strand may comprise a region that has a sequence that is substantially identical to the target gene sequence.

A double-stranded oligonucleotide compound (e.g. double-stranded RNA molecule) may include chemical modifications to ribonucleotides, including modifications to the ribose sugar, base, or backbone components of the ribonucleotides, such as those described herein or known in the art. Any such modifications, as used in a double-stranded RNA molecule (e.g. siRNA, shRNA, or the like), are encompassed by the term “double-stranded RNA” for the purposes of this disclosure.

In embodiments in which the oligonucleotide compound is double-stranded, a region of the antisense strand comprises a sequence that is substantially or fully complementary to a region of the target gene sequence (e.g. target mRNA). In such embodiments, the sense strand may comprise a sequence that is fully complementary to the sequence of the antisense strand. In other such embodiments, the sense strand may comprise a sequence that is substantially complementary to the sequence of the antisense strand, e.g. having 1, 2, 3, 4, or 5 mismatches in the duplex region formed by the sense and antisense strands. In certain embodiments, it is preferred that any mismatches occur within the terminal regions (e.g. within 6, 5, 4, 3, or 2 nucleotides of the 5′ and/or 3′ ends of the strands). In one embodiment, any mismatches in the duplex region formed from the sense and antisense strands occur within 6, 5, 4, 3, or 2 nucleotides of the 5′ end of the antisense strand.

In certain embodiments of the methods of the invention, the sense strand and antisense strand of the oligonucleotide compound may be two separate molecules that hybridize to form a duplex region but are otherwise unconnected. Such double-stranded RNA molecules formed from two separate strands are referred to as “small interfering RNAs” or “short interfering RNAs” (siRNAs). Thus, in some embodiments, the oligonucleotide compounds used in the methods of the invention comprise or consist of an siRNA.

In other embodiments, the sense strand and the antisense strand that hybridize to form a duplex region may be part of a single oligonucleotide, i.e. the sense and antisense strands are part of a self-complementary region of a single oligonucleotide. In such cases, the oligonucleotide compound comprises or consists of a single oligonucleotide that comprises a duplex region (also referred to as a stem region) and a loop region. The 3′ end of the sense strand is connected to the 5′ end of the antisense strand by a contiguous sequence of unpaired nucleotides, which will form the loop region. The loop region is typically of a sufficient length to allow the oligonucleotide to fold back on itself such that the antisense strand can base pair with the sense strand to form the duplex or stem region. The loop region can comprise from about 3 to about 25, from about 5 to about 15, or from about 8 to about 12 unpaired nucleotides. Such oligonucleotides (e.g. RNA molecules) with at least partially self-complementary regions are referred to as “short hairpin RNAs” (shRNAs). In certain embodiments, the oligonucleotide compounds used in the methods of the invention comprise or consist of a shRNA. The length of a single, at least partially self-complementary oligonucleotide can be from about 40 nucleotides to about 100 nucleotides, from about 45 nucleotides to about 85 nucleotides, or from about 50 nucleotides to about 60 nucleotides and comprise a duplex region and loop region each having the lengths recited herein.

In embodiments in which the oligonucleotide compound is double-stranded (e.g. comprises an siRNA), the sense strand typically comprises a sequence that is sufficiently complementary to the sequence of the antisense strand such that the two strands hybridize under physiological conditions to form a duplex region. A “duplex region” refers to the region in two complementary or substantially complementary oligonucleotides that form base pairs with one another, either by Watson-Crick base pairing or other hydrogen bonding interaction, to create a duplex between the two oligonucleotides. The duplex region of the oligonucleotide compound should be of sufficient length to allow the compound to enter the RNA interference pathway, e.g. by engaging the Dicer enzyme and/or the RISC complex. For instance, in some embodiments, the duplex region is about 15 to about 30 base pairs in length. Other lengths for the duplex region within this range are also suitable, such as about 15 to about 28 base pairs, about 15 to about 26 base pairs, about 15 to about 24 base pairs, about 15 to about 22 base pairs, about 17 to about 28 base pairs, about 17 to about 26 base pairs, about 17 to about 24 base pairs, about 17 to about 23 base pairs, about 17 to about 21 base pairs, about 19 to about 25 base pairs, about 19 to about 23 base pairs, or about 19 to about 21 base pairs. In certain embodiments, the duplex region is about 17 to about 24 base pairs in length. In other embodiments, the duplex region is about 19 to about 21 base pairs in length. In one embodiment, the duplex region is about 19 base pairs in length. In another embodiment, the duplex region is about 21 base pairs in length.

For embodiments in which the sense strand and antisense strand are two separate oligonucleotides (e.g. oligonucleotide compound comprises or consists of an siRNA), the sense strand and antisense strand need not be the same length as the length of the duplex region. For instance, one or both strands may be longer than the duplex region and have one or more unpaired nucleotides or mismatches flanking the duplex region. Thus, in some embodiments, the oligonucleotide compound (e.g. siRNA molecule) comprises at least one nucleotide overhang. As used herein, a “nucleotide overhang” refers to the unpaired nucleotide or nucleotides that extend beyond the duplex region at the terminal ends of the strands. Nucleotide overhangs are typically created when the 3′ end of one strand extends beyond the 5′ end of the other strand or when the 5′ end of one strand extends beyond the 3′ end of the other strand. The length of a nucleotide overhang is generally between 1 and 6 nucleotides, 1 and 5 nucleotides, 1 and 4 nucleotides, 1 and 3 nucleotides, 2 and 6 nucleotides, 2 and 5 nucleotides, or 2 and 4 nucleotides. In some embodiments, the nucleotide overhang comprises 1, 2, 3, 4, 5, or 6 nucleotides. In one particular embodiment, the nucleotide overhang comprises 1 to 4 nucleotides. In certain embodiments, the nucleotide overhang comprises 2 nucleotides. In certain other embodiments, the nucleotide overhang comprises a single nucleotide.

The nucleotides in the overhang can be ribonucleotides or modified nucleotides as described herein. In some embodiments, the nucleotides in the overhang are 2′-modified nucleotides (e.g. 2′-fluoro modified nucleotides, 2′-O-methyl modified nucleotides), deoxyribonucleotides, abasic nucleotides, inverted nucleotides (e.g. inverted abasic nucleotides, inverted deoxyribonucleotides), or combinations thereof. For instance, in one embodiment, the nucleotides in the overhang are deoxyribonucleotides, e.g. deoxythymidine. In another embodiment, the nucleotides in the overhang are 2′-O-methyl modified nucleotides, 2′-fluoro modified nucleotides, 2′-methoxyethyl modified nucleotides, or combinations thereof. In other embodiments, the overhang comprises a 5′-uridine-uridine-3′ (5′-UU-3′) dinucleotide. In such embodiments, the UU dinucleotide may comprise ribonucleotides or modified nucleotides, e.g. 2′-modified nucleotides. In other embodiments, the overhang comprises a 5′-deoxythymidine-deoxythymidine-3′ (5′-d′TdT-3′) dinucleotide. When a nucleotide overhang is present in the antisense strand, the nucleotides in the overhang can be complementary to the target gene sequence, form a mismatch with the target gene sequence, or comprise some other sequence (e.g. polypyrimidine or polypurine sequence, such as UU, TT, AA, GG, etc.).

The nucleotide overhang can be at the 5′ end or 3′ end of one or both strands. For example, in one embodiment, the oligonucleotide compound (e.g. siRNA molecule) comprises a nucleotide overhang at the 5′ end and the 3′ end of the antisense strand. In another embodiment, the oligonucleotide compound (e.g. siRNA molecule) comprises a nucleotide overhang at the 5′ end and the 3′ end of the sense strand. In some embodiments, the oligonucleotide compound (e.g. siRNA molecule) comprises a nucleotide overhang at the 5′ end of the sense strand and the 5′ end of the antisense strand. In other embodiments, the oligonucleotide compound (e.g. siRNA molecule) comprises a nucleotide overhang at the 3′ end of the sense strand and the 3′ end of the antisense strand.

The oligonucleotide compounds (e.g. siRNA molecules) used in the methods of the invention may comprise a single nucleotide overhang at one end of the double-stranded molecule and a blunt end at the other. A “blunt end” means that the sense strand and antisense strand are fully base-paired at the end of the molecule and there are no unpaired nucleotides that extend beyond the duplex region. In some embodiments, the oligonucleotide compound (e.g. siRNA molecule) comprises a nucleotide overhang at the 3′ end of the sense strand and a blunt end at the 5′ end of the sense strand and 3′ end of the antisense strand. In other embodiments, the oligonucleotide compound (e.g. siRNA molecule) comprises a nucleotide overhang at the 3′ end of the antisense strand and a blunt end at the 5′ end of the antisense strand and the 3′ end of the sense strand. In certain embodiments, the oligonucleotide compound (e.g. siRNA molecule) used in the methods of the invention comprises a blunt end at both ends of the double-stranded molecule. In such embodiments, the sense strand and antisense strand have the same length and the duplex region is the same length as the sense and antisense strands (i.e. the molecule is double-stranded over its entire length).

In embodiments in which the oligonucleotide compound comprises a sense strand and an antisense strand (e.g. the oligonucleotide compound comprises or consists of an siRNA molecule), the sense strand and antisense strand can each independently be about 15 to about 30 nucleotides in length, about 19 to about 30 nucleotides in length, about 18 to about 28 nucleotides in length, about 19 to about 27 nucleotides in length, about 19 to about 25 nucleotides in length, about 19 to about 23 nucleotides in length, about 19 to about 21 nucleotides in length, about 21 to about 25 nucleotides in length, or about 21 to about 23 nucleotides in length. In certain embodiments, the sense strand and antisense strand are each independently about 18, about 19, about 20, about 21, about 22, about 23, about 24, or about 25 nucleotides in length. In some embodiments, the sense strand and antisense strand have the same length but form a duplex region that is shorter than the strands such that the oligonucleotide compound has two nucleotide overhangs. For instance, in one embodiment, the oligonucleotide compound comprises (i) a sense strand and an antisense strand that are each 21 nucleotides in length, (ii) a duplex region that is 19 base pairs in length, and (iii) nucleotide overhangs of 2 unpaired nucleotides at both the 3′ end of the sense strand and the 3′ end of the antisense strand. In another embodiment, the oligonucleotide compound comprises (i) a sense strand and an antisense strand that are each 23 nucleotides in length, (ii) a duplex region that is 21 base pairs in length, and (iii) nucleotide overhangs of 2 unpaired nucleotides at both the 3′ end of the sense strand and the 3′ end of the antisense strand. In other embodiments, the sense strand and antisense strand have the same length and form a duplex region over their entire length such that there are no nucleotide overhangs on either end of the double-stranded molecule. In one such embodiment, the oligonucleotide compound is blunt ended (e.g. has two blunt ends) and comprises (i) a sense strand and an antisense strand, each of which is 21 nucleotides in length, and (ii) a duplex region that is 21 base pairs in length. In another such embodiment, the oligonucleotide compound is blunt ended (e.g. has two blunt ends) and comprises (i) a sense strand and an antisense strand, each of which is 23 nucleotides in length, and (ii) a duplex region that is 23 base pairs in length. In still another such embodiment, the oligonucleotide compound is blunt ended (e.g. has two blunt ends) and comprises (i) a sense strand and an antisense strand, each of which is 19 nucleotides in length, and (ii) a duplex region that is 19 base pairs in length.

In other embodiments of the methods of the invention, the sense strand or the antisense strand of the oligonucleotide compound is longer than the other strand and the two strands form a duplex region having a length equal to that of the shorter strand such that the oligonucleotide compound (e.g. siRNA molecule) comprises at least one nucleotide overhang. For example, in one embodiment, the oligonucleotide compound comprises (i) a sense strand that is 19 nucleotides in length, (ii) an antisense strand that is 21 nucleotides in length, (iii) a duplex region of 19 base pairs in length, and (iv) a nucleotide overhang of 2 unpaired nucleotides at the 3′ end of the antisense strand. In another embodiment, the oligonucleotide compound comprises (i) a sense strand that is 21 nucleotides in length, (ii) an antisense strand that is 23 nucleotides in length, (iii) a duplex region of 21 base pairs in length, and (iv) a nucleotide overhang of 2 unpaired nucleotides at the 3′ end of the antisense strand.

The oligonucleotide compounds used in the methods of the invention may comprise one or more modified nucleotides. A “modified nucleotide” refers to a nucleotide that has one or more chemical modifications to the nucleoside, nucleobase, pentose ring, or phosphate group. As used herein, modified nucleotides do not encompass ribonucleotides containing adenosine monophosphate, guanosine monophosphate, uridine monophosphate, and cytidine monophosphate. However, the oligonucleotide compounds may comprise combinations of modified nucleotides and ribonucleotides. Incorporation of modified nucleotides into oligonucleotide compounds can improve the in vivo stability of the oligonucleotide molecules, e.g., by reducing the molecules' susceptibility to nucleases and other degradation processes. The potency of oligonucleotide compounds for reducing expression of the target gene can also be enhanced by incorporation of modified nucleotides.

In certain embodiments, the modified nucleotides have a modification of the ribose sugar. These sugar modifications can include modifications at the 2′ and/or 5′ position of the pentose ring as well as bicyclic sugar modifications. A 2′-modified nucleotide refers to a nucleotide having a pentose ring with a substituent at the 2′ position other than OH. Such 2′-modifications include, but are not limited to, 2′-H (e.g. deoxyribonucleotides), 2′-O-alkyl (e.g. O—C₁-C₁₀ or O—C₁-C₁₀ substituted alkyl), 2′-O-allyl (O—CH₂CH═CH₂), 2′-C-allyl, 2′-deoxy-2′-fluoro (also referred to as 2′-F or 2′-fluoro), 2′-O-methyl (OCH₃), 2′-O-methoxyethyl (O—(CH₂)₂OCH₃), 2′-OCF₃, 2′-O(CH₂)₂SCH₃, 2′-O-aminoalkyl, 2′-amino (e.g. NH₂), 2′-O-ethylamine, and 2′-azido. Modifications at the 5′ position of the pentose ring include, but are not limited to, 5′-methyl (R or S); 5′-vinyl, and 5′-methoxy.

A “bicyclic sugar modification” refers to a modification of the pentose ring where a bridge connects two atoms of the ring to form a second ring resulting in a bicyclic sugar structure. In some embodiments the bicyclic sugar modification comprises a bridge between the 4′ and 2′ carbons of the pentose ring. Nucleotides comprising a sugar moiety with a bicyclic sugar modification are referred to herein as bicyclic nucleic acids or BNAs. Exemplary bicyclic sugar modifications include, but are not limited to, α-L-Methyleneoxy (4′-CH₂—O-2′) bicyclic nucleic acid (BNA); β-D-Methyleneoxy (4′-CH₂—O-2′) BNA (also referred to as a locked nucleic acid or LNA); Ethyleneoxy (4′-(CH₂) 2-O-2′) BNA; Aminooxy (4′-CH₂—O—N(R)-2′) BNA; Oxyamino (4′-CH₂—N(R)—O-2′) BNA; Methyl(methyleneoxy) (4′-CH(CH₃)—O-2′) BNA (also referred to as constrained ethyl or cEt); methylene-thio (4′-CH₂—S-2′) BNA; methylene-amino (4′-CH₂—N(R)-2′) BNA; methyl carbocyclic (4′-CH₂—CH(CH₃)-2′) BNA; propylene carbocyclic (4′-(CH₂) 3-2′) BNA; and Methoxy (ethyleneoxy) (4′-CH(CH₂OMe)-O-2′) BNA (also referred to as constrained MOE or cMOE). These and other sugar-modified nucleotides that can be incorporated into the oligonucleotide compounds used in the methods of the invention are described in U.S. Pat. No. 9,181,551, U.S. Patent Publication No. 2016/0122761, and Deleavey and Damha, Chemistry and Biology, Vol. 19:937-954, 2012, all of which are hereby incorporated by reference in their entireties.

In some embodiments, the oligonucleotide compounds comprise one or more 2′-fluoro modified nucleotides, 2′-O-methyl modified nucleotides, 2′-O-methoxyethyl modified nucleotides, 2′-O-alkyl modified nucleotides, 2′-O)-allyl modified nucleotides, bicyclic nucleic acids (BNAs), deoxyribonucleotides, or combinations thereof. In certain embodiments, the oligonucleotide compounds comprise one or more 2′-fluoro modified nucleotides, 2′-O-methyl modified nucleotides, 2′-O-methoxyethyl modified nucleotides, or combinations thereof. In one particular embodiment, the oligonucleotide compounds comprise one or more 2′-fluoro modified nucleotides, 2′-O-methyl modified nucleotides or combinations thereof. In another particular embodiment, the oligonucleotide compounds comprise one or more 2′-O-methoxyethyl modified nucleotides, BNAs, deoxyribonucleotides, or combinations thereof.

In embodiments in which the oligonucleotide compounds used in the methods of the invention comprise a sense strand and an antisense strand (e.g. oligonucleotide compounds comprise or consist of an siRNA), both the sense and antisense strands can comprise one or multiple modified nucleotides. For instance, in some embodiments, the sense strand comprises 1, 2, 3, 4, 5, 6, 7, 8, 9, 10 or more modified nucleotides. In certain embodiments, all nucleotides in the sense strand are modified nucleotides. In some embodiments, the antisense strand comprises 1, 2, 3, 4, 5, 6, 7, 8, 9, 10 or more modified nucleotides. In other embodiments, all nucleotides in the antisense strand are modified nucleotides. In certain other embodiments, all nucleotides in the sense strand and all nucleotides in the antisense strand are modified nucleotides. In these and other embodiments, the modified nucleotides can be 2′-fluoro modified nucleotides, 2′-O-methyl modified nucleotides, or combinations thereof.

In embodiments in which the oligonucleotide compounds used in the methods of the invention comprise or consist of a single-stranded antisense oligonucleotide, the antisense oligonucleotide can comprise 1, 2, 3, 4, 5, 6, 7, 8, 9, 10 or more modified nucleotides. In some embodiments, all the nucleotides in the single-stranded antisense oligonucleotide are modified nucleotides. In such embodiments, the single-stranded antisense oligonucleotide may be a gapmer oligonucleotide. A gapmer oligonucleotide comprises a 5′ terminal segment and a 3′ terminal segment, each terminal segment comprising 2 to 5 modified nucleotides (e g. 2′-O-methoxyethyl modified nucleotides or BNAs), wherein the terminal segments flank a center “gap” region comprising 8 to 10 deoxyribonucleotides. In one embodiment, the gapmer oligonucleotide comprises, in 5′ to 3′ order: 5 2′-O-methoxyethyl modified nucleotides, 10 deoxyribonucleotides, and 5 2′-O-methoxyethyl modified nucleotides. In another embodiment, the gapmer oligonucleotide comprises, in 5′ to 3′ order: 3 BNAs (e.g. LNAs), 10 deoxyribonucleotides, and 3 BNAs (e.g. LNAs).

In certain embodiments, the modified nucleotides incorporated into oligonucleotide compounds used in the methods of the invention have a modification of the nucleobase (also referred to herein as “base”). A “modified nucleobase” or “modified base” refers to a base other than the naturally occurring purine bases adenine (A) and guanine (G) and pyrimidine bases thymine (T), cytosine (C), and uracil (U). Modified nucleobases can be synthetic or naturally occurring modifications and include, but are not limited to, universal bases, 5-methylcytosine (5-me-C), 5-hydroxymethyl cytosine, xanthine (X), hypoxanthine (I), 2-aminoadenine, 6-methyladenine, 6-methylguanine, and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

In some embodiments, the modified base is a universal base. A “universal base” refers to a base analog that indiscriminately forms base pairs with all of the natural bases in RNA and DNA without altering the double helical structure of the resulting duplex region. Universal bases are known to those of skill in the art and include, but are not limited to, inosine, C-phenyl, C-naphthyl and other aromatic derivatives, azole carboxamides, and nitroazole derivatives, such as 3-nitropyrrole, 4-nitroindole, 5-nitroindole, and 6-nitroindole.

Other suitable modified bases that can be incorporated into the oligonucleotide compounds used in the methods of the invention include those described in Herdewijn, Antisense Nucleic Acid Drug Dev., Vol. 10:297-310, 2000 and Peacock et al., J. Org. Chem., Vol. 76:7295-7300, 2011, both of which are hereby incorporated by reference in their entireties. The skilled person is well aware that guanine, cytosine, adenine, thymine, and uracil may be replaced by other nucleobases, such as the modified nucleobases described above, without substantially altering the base pairing properties of an oligonucleotide comprising a nucleotide bearing such replacement nucleobase.

In some embodiments, the oligonucleotide compounds may comprise one or more abasic nucleotides. An “abasic nucleotide” or “abasic nucleoside” is a nucleotide or nucleoside that lacks a nucleobase at the 1′ position of the ribose sugar. In certain embodiments, the abasic nucleotides are incorporated into the terminal ends of the one or more oligonucleotides of the oligonucleotide compounds. For instance, in one embodiment in which the oligonucleotide compound comprises or consists of an siRNA, the sense strand comprises an abasic nucleotide as the terminal nucleotide at its 3′ end, its 5′ end, or both its 3′ and 5′ ends. In another embodiment, the antisense strand comprises an abasic nucleotide as the terminal nucleotide at its 3′ end, its 5′ end, or both its 3′ and 5′ ends. In such embodiments in which the abasic nucleotide is a terminal nucleotide, it may be an inverted nucleotide—that is, linked to the adjacent nucleotide through a 3′-3′ internucleotide linkage (when on the 3′ end of a strand) or through a 5′-5′ internucleotide linkage (when on the 5′ end of a strand) rather than the natural 3′-5′ internucleotide linkage. Abasic nucleotides may also comprise a sugar modification, such as any of the sugar modifications described above. In certain embodiments, abasic nucleotides comprise a 2′-modification, such as a 2′-fluoro modification, 2′-O-methyl modification, or a 2′-H (deoxy) modification. In one embodiment, the abasic nucleotide comprises a 2′-O-methyl modification. In another embodiment, the abasic nucleotide comprises a 2′-H modification (i.e. a deoxy abasic nucleotide).

The oligonucleotide compounds used in the methods of the invention may also comprise one or more modified internucleotide linkages. As used herein, the term “modified internucleotide linkage” refers to an internucleotide linkage other than the natural 3′ to 5′ phosphodiester linkage. In some embodiments, the modified internucleotide linkage is a phosphorous-containing internucleotide linkage, such as a phosphotriester, aminoalkylphosphotriester, an alkylphosphonate (e.g. methylphosphonate, 3′-alkylene phosphonate), a phosphinate, a phosphoramidate (e.g. 3′-amino phosphoramidate and aminoalkylphosphoramidate), a phosphorothioate (P═S), a chiral phosphorothioate, a phosphorodithioate, a thionophosphoramidate, a thionoalkylphosphonate, a thionoalkylphosphotriester, and a boranophosphate. In one embodiment, a modified internucleotide linkage is a 2′ to 5′ phosphodiester linkage. In other embodiments, the modified internucleotide linkage is a non-phosphorous-containing internucleotide linkage and thus can be referred to as a modified internucleoside linkage. Such non-phosphorous-containing linkages include, but are not limited to, morpholino linkages (formed in part from the sugar portion of a nucleoside); siloxane linkages (—O—Si(H)₂—O—); sulfide, sulfoxide and sulfone linkages; formacetyl and thioformacetyl linkages, alkene containing backbones; sulfamate backbones; methylenemethylimino (—CH₂—N(CH₃)—O—CH₂—) and methylenehydrazino linkages; sulfonate and sulfonamide linkages; amide linkages; and others having mixed N, O, S and CH₂ component parts. In one embodiment, the modified internucleoside linkage is a peptide-based linkage (e.g. aminoethylglycine) to create a peptide nucleic acid or PNA, such as those described in U.S. Pat. Nos. 5,539,082; 5,714,331; and 5,719,262. Other suitable modified internucleotide and internucleoside linkages that may be employed in the oligonucleotide compounds are described in U.S. Pat. Nos. 6,693,187, 9,181,551, U.S. Patent Publication No. 2016/0122761, and Deleavey and Damha, Chemistry and Biology, Vol. 19:937-954, 2012, all of which are hereby incorporated by reference in their entireties.

In certain embodiments, the oligonucleotide compounds used in the methods of the invention comprise one or more phosphorothioate internucleotide linkages. In some embodiments, the oligonucleotide compounds comprise 1, 2, 3, 4, 5, 6, 7, 8, 9, 10, or more phosphorothioate internucleotide linkages. In embodiments in which the oligonucleotide compounds are double-stranded (e.g. the oligonucleotide compound comprises an siRNA), the phosphorothioate internucleotide linkages may be present in the sense strand, antisense strand, or both strands of the oligonucleotide compounds. For instance, in some embodiments, the sense strand comprises 1, 2, 3, 4, 5, 6, 7, 8, or more phosphorothioate internucleotide linkages. In other embodiments, the antisense strand comprises 1, 2, 3, 4, 5, 6, 7, 8, or more phosphorothioate internucleotide linkages. In still other embodiments, both strands comprise 1, 2, 3, 4, 5, 6, 7, 8, or more phosphorothioate internucleotide linkages. The oligonucleotide compounds can comprise one or more phosphorothioate internucleotide linkages at the 3′-end, the 5′-end, or both the 3′- and 5′-ends of the sense strand, the antisense strand, or both strands. For instance, in certain embodiments, the oligonucleotide compound comprises about 1 to about 6 or more (e.g., about 1, 2, 3, 4, 5, 6 or more) consecutive phosphorothioate internucleotide linkages at the 3′-end of the sense strand, the antisense strand, or both strands. In other embodiments, the oligonucleotide compound comprises about 1 to about 6 or more (e.g., about 1, 2, 3, 4, 5, 6 or more) consecutive phosphorothioate internucleotide linkages at the 5′-end of the sense strand, the antisense strand, or both strands. In one particular embodiment, the antisense strand comprises at least 1 but no more than 6 phosphorothioate internucleotide linkages and the sense strand comprises at least 1 but no more than 4 phosphorothioate internucleotide linkages. In another particular embodiment, the antisense strand comprises at least 1 but no more than 4 phosphorothioate internucleotide linkages and the sense strand comprises at least 1 but no more than 2 phosphorothioate internucleotide linkages.

In some embodiments, the oligonucleotide compound comprises a single phosphorothioate internucleotide linkage between the terminal nucleotides at the 3′ end of the sense strand. In other embodiments, the oligonucleotide compound comprises two consecutive phosphorothioate internucleotide linkages between the terminal nucleotides at the 3′ end of the sense strand. In one embodiment, the oligonucleotide compound comprises a single phosphorothioate internucleotide linkage between the terminal nucleotides at the 3′ end of the sense strand and a single phosphorothioate internucleotide linkage between the terminal nucleotides at the 3′ end of the antisense strand. In another embodiment, the oligonucleotide compound comprises two consecutive phosphorothioate internucleotide linkages between the terminal nucleotides at the 3′ end of the antisense strand (i.e. a phosphorothioate internucleotide linkage at the first and second internucleotide linkages at the 3′ end of the antisense strand). In another embodiment, the oligonucleotide compound comprises two consecutive phosphorothioate internucleotide linkages between the terminal nucleotides at both the 3′ and 5′ ends of the antisense strand. In yet another embodiment, the oligonucleotide compound comprises two consecutive phosphorothioate internucleotide linkages between the terminal nucleotides at both the 3′ and 5′ ends of the antisense strand and two consecutive phosphorothioate internucleotide linkages at the 5′ end of the sense strand. In still another embodiment, the oligonucleotide compound comprises two consecutive phosphorothioate internucleotide linkages between the terminal nucleotides at both the 3′ and 5′ ends of the antisense strand and two consecutive phosphorothioate internucleotide linkages between the terminal nucleotides at the 3′ end of the sense strand. In another embodiment, the oligonucleotide compound comprises two consecutive phosphorothioate internucleotide linkages between the terminal nucleotides at both the 3′ and 5′ ends of the antisense strand and two consecutive phosphorothioate internucleotide linkages between the terminal nucleotides at both the 3′ and 5′ ends of the sense strand (i.e. a phosphorothioate internucleotide linkage at the first and second internucleotide linkages at both the 5′ and 3′ ends of the antisense strand and a phosphorothioate internucleotide linkage at the first and second internucleotide linkages at both the 5′ and 3′ ends of the sense strand). In yet another embodiment, the oligonucleotide compound comprises two consecutive phosphorothioate internucleotide linkages between the terminal nucleotides at both the 3′ and 5′ ends of the antisense strand and a single phosphorothioate internucleotide linkage between the terminal nucleotides at the 3′ end of the sense strand. In any of the embodiments in which one or both strands comprise one or more phosphorothioate internucleotide linkages, the remaining internucleotide linkages within the strands can be the natural 3′ to 5′ phosphodiester linkages. For instance, in some embodiments, each internucleotide linkage of the sense and antisense strands is selected from phosphodiester and phosphorothioate, wherein at least one internucleotide linkage is a phosphorothioate. Likewise, in embodiments in which the oligonucleotide compound comprises or consists of a single oligonucleotide (e.g. a single-stranded antisense oligonucleotide), each internucleotide linkage in the oligonucleotide is selected from phosphodiester and phosphorothioate, wherein at least one internucleotide linkage is a phosphorothioate. In other embodiments, all internucleotide linkages in the single-stranded oligonucleotide are phosphorothioate internucleotide linkages.

In embodiments in which the oligonucleotide compound comprises a nucleotide overhang, two or more of the unpaired nucleotides in the overhang can be connected by a phosphorothioate internucleotide linkage. In certain embodiments, all the unpaired nucleotides in a nucleotide overhang at the 3′ end of the antisense strand and/or the sense strand are connected by phosphorothioate internucleotide linkages. In other embodiments, all the unpaired nucleotides in a nucleotide overhang at the 5′ end of the antisense strand and/or the sense strand are connected by phosphorothioate internucleotide linkages. In still other embodiments, all the unpaired nucleotides in any nucleotide overhang are connected by phosphorothioate internucleotide linkages.

The modified nucleotides that can be incorporated into the oligonucleotide compounds used in the methods of the invention may have more than one chemical modification described herein. For instance, the modified nucleotide may have a modification to the ribose sugar as well as a modification to the nucleobase. By way of example, a modified nucleotide may comprise a 2′ sugar modification (e.g. 2′-fluoro, 2′-O-methyl, 2′-O-methoxyethyl, or BNA) and comprise a modified base (e.g. 5-methyl cytosine or pseudouracil). In other embodiments, the modified nucleotide may comprise a sugar modification in combination with a modification to the 5′ phosphate that would create a modified internucleotide or internucleoside linkage when the modified nucleotide was incorporated into a polynucleotide. For instance, in some embodiments, the modified nucleotide may comprise a sugar modification, such as a 2′-fluoro modification, a 2′-O-methyl modification, 2′-O-methoxyethyl modification, or a bicyclic sugar modification, as well as a 5′ phosphorothioate group. Accordingly, in some embodiments, one or both oligonucleotides of the oligonucleotide compounds used in the methods of the invention comprise a combination of 2′ modified nucleotides or BNAs and phosphorothioate internucleotide linkages. In certain embodiments, both the sense and antisense strands of the double-stranded oligonucleotide compounds comprise a combination of 2′-fluoro modified nucleotides, 2′-O-methyl modified nucleotides, and phosphorothioate internucleotide linkages.

The oligonucleotide compounds used in the methods of the invention can readily be made using techniques known in the art, for example, using conventional nucleic acid solid phase synthesis. The oligonucleotides of the oligonucleotide compounds can be assembled on a suitable nucleic acid synthesizer utilizing standard nucleotide or nucleoside precursors (e.g. phosphoramidites). Automated nucleic acid synthesizers are sold commercially by several vendors, including DNA/RNA synthesizers from Applied Biosystems (Foster City, CA), MerMade synthesizers from BioAutomation (Irving, TX), and OligoPilot synthesizers from GE Healthcare Life Sciences (Pittsburgh, PA).

A 2′ silyl protecting group can be used in conjunction with acid labile dimethoxytrityl (DMT) at the 5′ position of ribonucleosides to synthesize oligonucleotides via phosphoramidite chemistry. Final deprotection conditions are known not to significantly degrade RNA products. All syntheses can be conducted in any automated or manual synthesizer on large, medium, or small scale. The syntheses may also be carried out in multiple well plates, columns, or glass slides.

The 2′-O-silyl group can be removed via exposure to fluoride ions, which can include any source of fluoride ion, e.g., those salts containing fluoride ion paired with inorganic counterions e.g., cesium fluoride and potassium fluoride or those salts containing fluoride ion paired with an organic counterion, e.g., a tetraalkylammonium fluoride. A crown ether catalyst can be utilized in combination with the inorganic fluoride in the deprotection reaction. Preferred fluoride ion sources are tetrabutylammonium fluoride or aminohydrofluorides (e.g., combining aqueous HF with triethylamine in a dipolar aprotic solvent, e.g., dimethylformamide).

The choice of protecting groups for use on the phosphite triesters and phosphotriesters can alter the stability of the triesters towards fluoride. Methyl protection of the phosphotriester or phosphitetriester can stabilize the linkage against fluoride ions and improve process yields.

Since ribonucleosides have a reactive 2′ hydroxyl substituent, it can be desirable to protect the reactive 2′ position in RNA with a protecting group that is orthogonal to a 5′-O-dimethoxytrityl protecting group, e.g., one stable to treatment with acid. Silyl protecting groups meet this criterion and can be readily removed in a final fluoride deprotection step that can result in minimal RNA degradation.

Tetrazole catalysts can be used in the standard phosphoramidite coupling reaction. Preferred catalysts include, e.g., tetrazole, S-ethyl-tetrazole, benzylthiotetrazole, p-nitrophenyltetrazole.

As can be appreciated by the skilled artisan, further methods of synthesizing the oligonucleotide compounds described herein will be evident to those of ordinary skill in the art. Additionally, the various synthetic steps may be performed in an alternate sequence or order to give the desired compounds. Other synthetic chemistry transformations, protecting groups (e.g., for hydroxyl, amino, etc. present on the bases) and protecting group methodologies (protection and deprotection) useful in synthesizing the oligonucleotide compounds described herein are known in the art and include, for example, the methods described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. Custom synthesis of oligonucleotide compounds is also available from several commercial vendors, including Dharmacon, Inc. (Lafayette, CO), AxoLabs GmbH (Kulmbach, Germany), and Ambion, Inc. (Foster City, CA).

In certain embodiments of the methods of the invention, the oligonucleotide compounds are covalently attached to a ligand. As used herein, a “ligand” refers to any compound or molecule that binds specifically and reversibly to another compound or molecule to form a complex. The interaction of a ligand with another compound or molecule may elicit a biological response (e.g. initiate a signal transduction cascade, induce receptor-mediated endocytosis) or may just be a physical association. In some embodiments, the ligand is a ligand of a receptor expressed on the surface of a cell, such as a cell to which the oligonucleotide compound is intended to be specifically delivered. The ligand can modify one or more properties of the oligonucleotide compound to which it is attached, such as the pharmacodynamic, pharmacokinetic, binding, absorption, cellular distribution, cellular uptake, charge and/or clearance properties of the oligonucleotide compound.

The ligand may comprise a serum protein (e.g., human serum albumin, low-density lipoprotein, globulin), a cholesterol moiety, a vitamin (biotin, vitamin E, vitamin B₁₂), a folate moiety, a steroid, a bile acid (e.g. cholic acid), a fatty acid (e.g., palmitic acid, myristic acid), a carbohydrate (e.g., a dextran, pullulan, chitin, chitosan, inulin, cyclodextrin or hyaluronic acid), a glycoside, a phospholipid, or antibody or binding fragment thereof (e.g. antibody or binding fragment that targets the oligonucleotide compound to a specific cell type, such as liver). Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., adamantane acetic acid, 1-pyrene butyric acid, dihydrotestosterone, 1,3-Bis-O (hexadecyl)glycerol, geranyloxyhexyl group, hexadecylglycerol, borneol, menthol, 1,3-propanediol, heptadecyl group, O3-(oleoyl) lithocholic acid, O3-(oleoyl) cholenic acid, dimethoxytrityl, or phenoxazine), peptides (e.g., antennapedia peptide, Tat peptide, RGD peptides), alkylating agents, polymers, such as polyethylene glycol (PEG) (e.g., PEG-40K), polyamino acids, and polyamines (e.g. spermine, spermidine).

In some embodiments, the ligand comprises a lipid or other hydrophobic molecule. In one embodiment, the ligand comprises a cholesterol moiety or other steroid. Cholesterol-conjugated oligonucleotides have been reported to be more active than their unconjugated counterparts (Manoharan, Antisense Nucleic Acid Drug Development, Vol. 12:103-228, 2002). Ligands comprising cholesterol moieties and other lipids for conjugation to nucleic acid molecules have also been described in U.S. Pat. Nos. 7,851,615; 7,745,608; and 7,833,992, all of which are hereby incorporated by reference in their entireties. In another embodiment, the ligand comprises a folate moiety. Oligonucleotides conjugated to folate moieties can be taken up by cells via a receptor-mediated endocytosis pathway. Such folate-oligonucleotide conjugates are described in U.S. Pat. No. 8,188,247, which is hereby incorporated by reference in its entirety.

In certain embodiments, the ligand specifically binds to a receptor or other protein expressed on the surface of a target cell to which the oligonucleotide compound is intended to be delivered. In some such embodiments, the ligand is an antibody or antigen-binding fragment thereof (e.g. Fab, scFv) that specifically binds to a cell surface receptor, such as the asialoglycoprotein receptor (ASGPR) or low-density lipoprotein (LDL) receptor for delivery to the liver, the transferrin receptor for delivery to skeletal muscle, cardiac muscle, and central nervous system, and epidermal growth factor receptor for delivery to tumor tissue.

In some embodiments of the methods of the invention, the oligonucleotide compound is covalently attached to a ligand of a receptor expressed on the surface of a liver cell, such as a hepatocyte. In one such embodiment, the oligonucleotide compound is covalently attached to a ligand that binds to the ASGPR or component thereof (e.g. ASGR1, ASGR2). In one particular embodiment, the ligand comprises an antibody or binding fragment thereof that specifically binds to ASGR1 and/or ASGR2. In another embodiment, the ligand comprises a Fab fragment of an antibody that specifically binds to ASGR1 and/or ASGR2. A “Fab fragment” is comprised of one immunoglobulin light chain (i.e. light chain variable region (VL) and constant region (CL)) and the CHI region and variable region (VH) of one immunoglobulin heavy chain. In another embodiment, the ligand comprises a single-chain variable antibody fragment (scFv fragment) of an antibody that specifically binds to ASGR1 and/or ASGR2. An “scFv fragment” comprises the VH and VL regions of an antibody, wherein these regions are present in a single polypeptide chain, and optionally comprising a peptide linker between the VH and VL regions that enables the Fv to form the desired structure for antigen binding. Exemplary antibodies and binding fragments thereof that specifically bind to ASGR1 that can be used as ligands for targeting the oligonucleotide compounds to the liver are described in WIPO Publication No. WO 2017/058944, which is hereby incorporated by reference in its entirety. Other antibodies or binding fragments thereof that specifically bind to ASGR1, LDL receptor, or other liver surface-expressed proteins suitable for use as ligands that can be covalently attached to the oligonucleotide compounds used in the methods of the invention are commercially available.

In certain embodiments, the ligand comprises a carbohydrate. A “carbohydrate” refers to a compound made up of one or more monosaccharide units having at least 6 carbon atoms (which can be linear, branched or cyclic) with an oxygen, nitrogen or sulfur atom bonded to each carbon atom. Carbohydrates include, but are not limited to, the sugars (e.g., monosaccharides, disaccharides, trisaccharides, tetrasaccharides, and oligosaccharides containing from about 4, 5, 6, 7, 8, or 9 monosaccharide units), and polysaccharides, such as starches, glycogen, cellulose and polysaccharide gums. In some embodiments, the carbohydrate incorporated into the ligand is a monosaccharide selected from a pentose, hexose, or heptose and di- and tri-saccharides including such monosaccharide units. In other embodiments, the carbohydrate incorporated into the ligand is an amino sugar, such as galactosamine, glucosamine, N-acetylgalactosamine, and N-acetylglucosamine.

In some embodiments, the ligand comprises a hexose or hexosamine. The hexose may be selected from glucose, galactose, mannose, fucose, or fructose. The hexosamine may be selected from fructosamine, galactosamine, glucosamine, or mannosamine. In certain embodiments, the ligand comprises glucose, galactose, galactosamine, or glucosamine. In one embodiment, the ligand comprises glucose, glucosamine, or N-acetylglucosamine. In another embodiment, the ligand comprises galactose, galactosamine, or N-acetyl-galactosamine. In particular embodiments, the ligand comprises N-acetyl-galactosamine. Ligands comprising glucose, galactose, and N-acetyl-galactosamine (GalNAc) are particularly effective in targeting compounds to liver cells because such ligands bind to the ASGPR expressed on the surface of hepatocytes. See, e.g., D'Souza and Devarajan, J. Control Release, Vol. 203:126-139, 2015. Examples of GalNAc- or galactose-containing ligands that can be covalently attached to an oligonucleotide of the oligonucleotide compounds are described in U.S. Pat. Nos. 7,491,805; 8,106,022; and 8,877,917; U.S. Patent Publication No. 20030130186; and WIPO Publication No. WO 2013166155, all of which are hereby incorporated by reference in their entireties.

In certain embodiments, the ligand comprises a multivalent carbohydrate moiety. As used herein, a “multivalent carbohydrate moiety” refers to a moiety comprising two or more carbohydrate units capable of independently binding or interacting with other molecules. For example, a multivalent carbohydrate moiety comprises two or more binding domains comprised of carbohydrates that can bind to two or more different molecules or two or more different sites on the same molecule. The valency of the carbohydrate moiety denotes the number of individual binding domains within the carbohydrate moiety. For instance, the terms “monovalent,” “bivalent,” “trivalent,” and “tetravalent” with reference to the carbohydrate moiety refer to carbohydrate moieties with one, two, three, and four binding domains, respectively. The multivalent carbohydrate moiety may comprise a multivalent lactose moiety, a multivalent galactose moiety, a multivalent glucose moiety, a multivalent N-acetyl-galactosamine moiety, a multivalent N-acetyl-glucosamine moiety, a multivalent mannose moiety, or a multivalent fucose moiety. In some embodiments, the ligand comprises a multivalent galactose moiety. In other embodiments, the ligand comprises a multivalent N-acetyl-galactosamine moiety. In these and other embodiments, the multivalent carbohydrate moiety can be bivalent, trivalent, or tetravalent. In such embodiments, the multivalent carbohydrate moiety can be bi-antennary or tri-antennary. In one particular embodiment, the multivalent N-acetyl-galactosamine moiety is trivalent or tetravalent. In another particular embodiment, the multivalent galactose moiety is trivalent or tetravalent. Exemplary trivalent and tetravalent GalNAc-containing ligands for covalent attachment to oligonucleotide compounds for use in the methods of the invention are described in detail below.

The ligand can be covalently attached or conjugated to the oligonucleotide compound directly or indirectly. For instance, in some embodiments in which the oligonucleotide compound is double-stranded (e.g. the oligonucleotide compound comprises an siRNA), the ligand is covalently attached directly to the sense or antisense strand of the oligonucleotide compound. In other embodiments, the ligand is covalently attached via a linker to the sense or antisense strand of the oligonucleotide compound. The ligand can be attached to nucleobases, sugar moieties, or internucleotide linkages of oligonucleotides comprised in the oligonucleotide compounds used in the methods of the invention. Conjugation or attachment to purine nucleobases or derivatives thereof can occur at any position including, endocyclic and exocyclic atoms. In certain embodiments, the 2-, 6-, 7-, or 8-positions of a purine nucleobase are attached to a ligand. Conjugation or attachment to pyrimidine nucleobases or derivatives thereof can also occur at any position. In some embodiments, the 2-, 5-, and 6-positions of a pyrimidine nucleobase can be attached to a ligand. Conjugation or attachment to sugar moieties of nucleotides can occur at any carbon atom. Exemplary carbon atoms of a sugar moiety that can be attached to a ligand include the 2′, 3′, and 5′ carbon atoms. The 1′ position can also be attached to a ligand, such as in an abasic nucleotide. Internucleotide linkages can also support ligand attachments. For phosphorus-containing linkages (e.g., phosphodiester, phosphorotbioate, phosphorodithiotate, phosphoroamidate, and the like), the ligand can be attached directly to the phosphorus atom or to an O, N, or S atom bound to the phosphorus atom. For amine- or amide-containing internucleoside linkages (e.g., PNA), the ligand can be attached to the nitrogen atom of the amine or amide or to an adjacent carbon atom.

In some embodiments, the ligand may be attached to the 3′ or 5′ end of a single-stranded oligonucleotide compound (e.g. a single-stranded antisense oligonucleotide compound). In embodiments in which the oligonucleotide compound is double-stranded (e.g. the oligonucleotide compound comprises an siRNA), the ligand may be attached to the 3′ or 5′ end of either the sense or antisense strand. In certain embodiments, the ligand is covalently attached to the 5′ end of the sense strand. In such embodiments, the ligand is attached to the 5′-terminal nucleotide of the sense strand. In these and other embodiments, the ligand is attached at the 5′-position of the 5′-terminal nucleotide of the sense strand. In other embodiments, the ligand is covalently attached to the 3′ end of the sense strand. For example, in some embodiments, the ligand is attached to the 3′-terminal nucleotide of the sense strand. In certain such embodiments, the ligand is attached at the 3′-position of the 3′-terminal nucleotide of the sense strand. In alternative embodiments, the ligand is attached near the 3′ end of the sense strand, but before one or more terminal nucleotides (i.e. before 1, 2, 3, or 4 terminal nucleotides). In some embodiments, the ligand is attached at the 2′-position of the sugar of the 3′-terminal nucleotide of the sense strand. In other embodiments, the ligand is attached at the 2′-position of the sugar of the 5′-terminal nucleotide of the sense strand.

In certain embodiments, the ligand is attached to the oligonucleotide compound via a linker. A “linker” is an atom or group of atoms that covalently joins a ligand to an oligonucleotide component of the oligonucleotide compound. The linker may be from about 1 to about 30 atoms in length, from about 2 to about 28 atoms in length, from about 3 to about 26 atoms in length, from about 4 to about 24 atoms in length, from about 6 to about 20 atoms in length, from about 7 to about 20 atoms in length, from about 8 to about 20 atoms in length, from about 8 to about 18 atoms in length, from about 10 to about 18 atoms in length, and from about 12 to about 18 atoms in length. In some embodiments, the linker may comprise a bifunctional linking moiety, which generally comprises an alkyl moiety with two functional groups One of the functional groups is selected to bind to the compound of interest (e.g. an oligonucleotide of the oligonucleotide compound) and the other is selected to bind essentially any selected group, such as a ligand as described herein. In certain embodiments, the linker comprises a chain structure or an oligomer of repeating units, such as ethylene glycol or amino acid units. Examples of functional groups that are typically employed in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like.

Linkers that may be used to attach a ligand to an oligonucleotide in the oligonucleotide compounds used in the methods of the invention include, but are not limited to, pyrrolidine, 8-amino-3,6-dioxaoctanoic acid, succinimidyl 4-(N-maleimidomethyl)cyclohexane-1-carboxylate, 6-aminohexanoic acid, substituted C₁-C₁₀ alkyl, substituted or unsubstituted C₂-C₁₀ alkenyl or substituted or unsubstituted C₂-C₁₀ alkynyl. Preferred substituent groups for such linkers include, but are not limited to, hydroxyl, amino, alkoxy, carboxy, benzyl, phenyl, nitro, thiol, thioalkoxy, halogen, alkyl, aryl, alkenyl and alkynyl.

In certain embodiments, the linkers are cleavable. A cleavable linker is one which is sufficiently stable outside the cell, but which upon entry into a target cell is cleaved to release the two parts the linker is holding together. In some embodiments, the cleavable linker is cleaved at least 10 times, 20 times, 30 times, 40 times, 50 times, 60 times, 70 times, 80 times, 90 times, or more, or at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linkers are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linker by reduction; esterases; endosomes or agents that can create an acidic environment, e.g., those that result in a pH of five or lower; enzymes that can hydrolyze or degrade an acid cleavable linker by acting as a general acid, peptidases (which can be substrate specific), and phosphatases.

A cleavable linker may comprise a moiety that is susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable group that is cleaved at a preferred pH, thereby releasing the oligonucleotide compound from the ligand inside the cell, or into the desired compartment of the cell.

A linker can include a cleavable group that is cleavable by a particular enzyme. The type of cleavable group incorporated into a linker can depend on the cell to be targeted. For example, liver-targeting ligands can be linked to oligonucleotide compounds through a linker that includes an ester group. Liver cells are rich in esterases, and therefore the linker will be cleaved more efficiently in liver cells than in cell types that are not esterase-rich. Other types of cells rich in esterases include cells of the lung, renal cortex, and testis. Linkers that contain peptide bonds can be used when targeting cells rich in peptidases, such as liver cells and synoviocytes.

In general, the suitability of a candidate cleavable linker can be evaluated by testing the ability of a degradative agent (or condition) to cleave the candidate linker. It will also be desirable to also test the candidate cleavable linker for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In some embodiments, useful candidate linkers are cleaved at least 2, 4, 10, 20, 50, 70, or 100 times faster in the cell (or under in vitro conditions selected to mimic intracellular conditions) as compared to blood or serum (or under in vitro conditions selected to mimic extracellular conditions).

In other embodiments, redox cleavable linkers are utilized. Redox cleavable linkers are cleaved upon reduction or oxidation. An example of a reductively cleavable group is a disulfide linking group (—S—S—). To determine if a candidate cleavable linker is a suitable “reductively cleavable linker,” or for example is suitable for use with a particular oligonucleotide compound and particular ligand, one can use one or more methods described herein. For example, a candidate linker can be evaluated by incubation with dithiothreitol (DTT), or other reducing agent known in the art, which mimics the rate of cleavage that would be observed in a cell, e.g., a target cell. The candidate linkers can also be evaluated under conditions which are selected to mimic blood or serum conditions. In a specific embodiment, candidate linkers are cleaved by at most 10% in the blood.

In yet other embodiments, phosphate-based cleavable linkers, which are cleaved by agents that degrade or hydrolyze the phosphate group, are employed to covalently attach a ligand to an oligonucleotide of the oligonucleotide compound. An example of an agent that hydrolyzes phosphate groups in cells are enzymes, such as phosphatases in cells. Examples of phosphate-based cleavable groups are —O—P(O)(ORk)-O—, —O—P(S)(ORk)-O—, —O—P(S)(SRk)-O—, —S—P(O) (ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, and —O—P(S)(Rk)-S—, where Rk can be hydrogen or alkyl. Specific embodiments include —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—; —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—. Another specific embodiment is —O—P(O)(OH)—O—. These candidate linkers can be evaluated using methods analogous to those described above.

In other embodiments, the linkers may comprise acid cleavable groups, which are groups that are cleaved under acidic conditions. In some embodiments, acid cleavable groups are cleaved in an acidic environment with a pH of about 6.5 or lower (e.g., about 6.0, 5.5, 5.0, or lower), or by agents, such as enzymes that can act as a general acid. In a cell, specific low pH organelles, such as endosomes and lysosomes, can provide a cleaving environment for acid cleavable groups. Examples of acid cleavable linking groups include, but are not limited to, hydrazones, esters, and esters of amino acids. Acid cleavable groups can have the general formula —C═NN—, C(O)O, or —OC(O). A specific embodiment is when the carbon attached to the oxygen of the ester (the alkoxy group) is an aryl group, substituted alkyl group, or tertiary alkyl group such as dimethyl, pentyl or t-butyl. These candidates can be evaluated using methods analogous to those described above.

In other embodiments, the linkers may comprise ester-based cleavable groups, which are cleaved by enzymes, such as esterases and amidases in cells. Examples of ester-based cleavable groups include, but are not limited to, esters of alkylene, alkenylene and alkynylene groups. Ester cleavable groups have the general formula —C(O)O—, or —OC(O)—. These candidate linkers can be evaluated using methods analogous to those described above.

In further embodiments, the linkers may comprise peptide-based cleavable groups, which are cleaved by enzymes, such as peptidases and proteases in cells. Peptide-based cleavable groups are peptide bonds formed between amino acids to yield oligopeptides (e.g., dipeptides, tripeptides etc.) and polypeptides. Peptide-based cleavable groups include the amide group (—C(O)NH—). The amide group can be formed between any alkylene, alkenylene or alkynylene. A peptide bond is a special type of amide bond formed between amino acids to yield peptides and proteins. The peptide-based cleavage group is generally limited to the peptide bond (i.e., the amide bond) formed between amino acids yielding peptides and proteins. Peptide-based cleavable linking groups have the general formula —NHCHR^{A}C(O)NHCHR^{B}C(O)—, where RA and RB are the side chains of the two adjacent amino acids. These candidates can be evaluated using methods analogous to those described above.

Other types of linkers suitable for attaching ligands to an oligonucleotide of the oligonucleotide compounds used in the methods of the invention are known in the art and can include the linkers described in U.S. Pat. Nos. 7,723,509; 8,017,762; 8,828,956; 8,877,917; and 9,181,551, all of which are hereby incorporated by reference in their entireties.

In certain embodiments, the ligand covalently attached to an oligonucleotide of the oligonucleotide compound comprises a GalNAc moiety, e.g., a multivalent GalNAc moiety. In some embodiments, the multivalent GalNAc moiety is a trivalent GalNAc moiety and is attached to the 3′ end of the oligonucleotide (e.g. sense strand in double-stranded oligonucleotide compounds). In other embodiments, the multivalent GalNAc moiety is a trivalent GalNAc moiety and is attached to the 5′ end of the oligonucleotide (e.g. sense strand in double-stranded oligonucleotide compounds). In yet other embodiments, the multivalent GalNAc moiety is a tetravalent GalNAc moiety and is attached to the 3′ end of the oligonucleotide (e.g. sense strand in double-stranded oligonucleotide compounds). In still other embodiments, the multivalent GalNAc moiety is a tetravalent GalNAc moiety and is attached to the 5′ end of the oligonucleotide (e.g. sense strand in double-stranded oligonucleotide compounds).

Exemplary trivalent and tetravalent GalNAc moieties and linkers that can be attached to an oligonucleotide of the oligonucleotide compounds used in the methods of the invention are provided in the structural formulas I-IX below. “Ac” in the formulas listed herein represents an acetyl group.

In one embodiment, the oligonucleotide compound comprises a ligand and linker having the following structure of Formula I, wherein each n is independently 1 to 3, k is 1 to 3, m is 1 or 2, j is 1 or 2, and the ligand is attached to the 3′ end of an oligonucleotide of the oligonucleotide compound (e.g. sense strand in a double-stranded oligonucleotide compound) (represented by the solid wavy line):

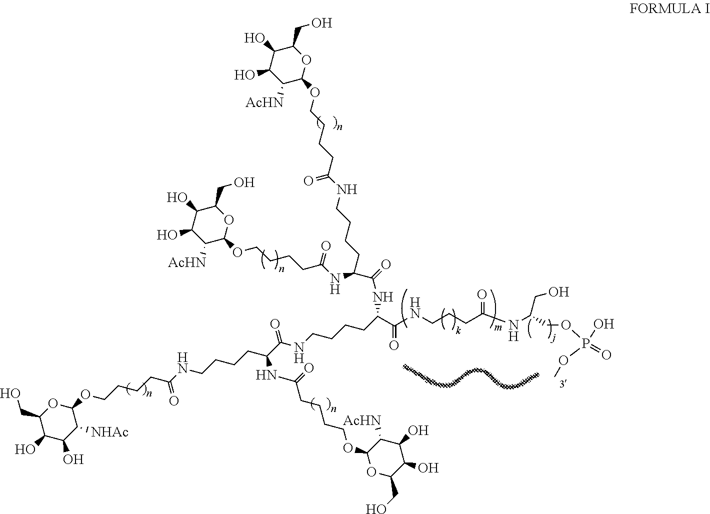

In another embodiment, the oligonucleotide compound comprises a ligand and linker having the following structure of Formula II, wherein each n is independently 1 to 3, k is 1 to 3, m is 1 or 2, j is 1 or 2, and the ligand is attached to the 3′ end of an oligonucleotide of the oligonucleotide compound (e.g. sense strand in a double-stranded oligonucleotide compound) (represented by the solid wavy line):

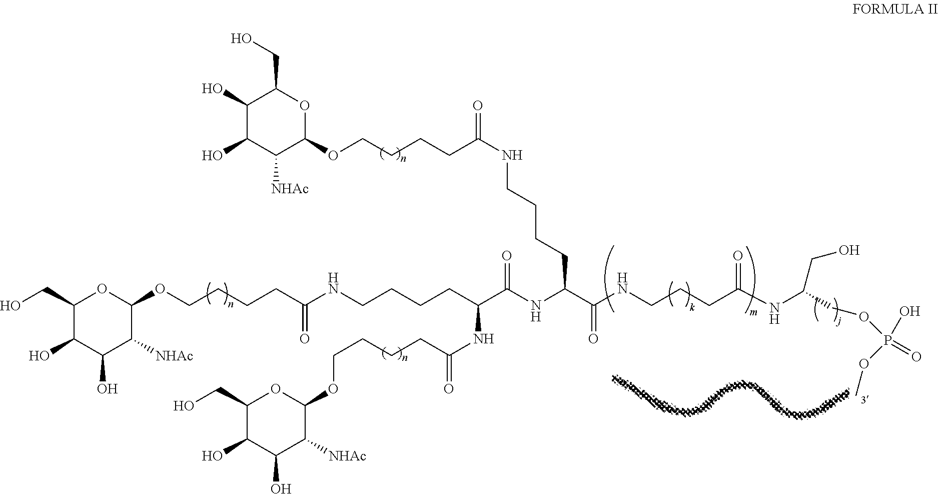

In yet another embodiment, the oligonucleotide compound comprises a ligand and linker having the following structure of Formula III, wherein the ligand is attached to the 3′ end of an oligonucleotide of the oligonucleotide compound (e.g. sense strand in a double-stranded oligonucleotide compound) (represented by the solid wavy line):

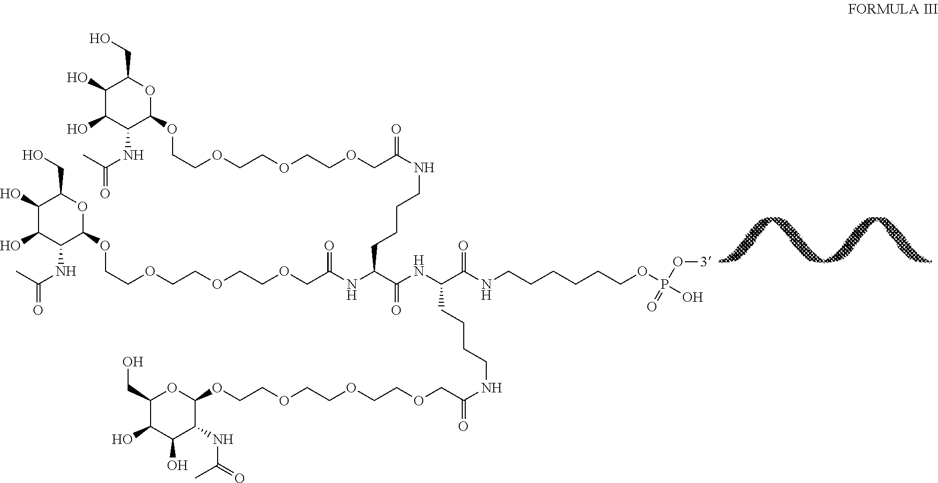

In still another embodiment, the oligonucleotide compound comprises a ligand and linker having the following structure of Formula IV, wherein the ligand is attached to the 3′ end of an oligonucleotide of the oligonucleotide compound (e.g. sense strand in a double-stranded oligonucleotide compound) (represented by the solid wavy line):

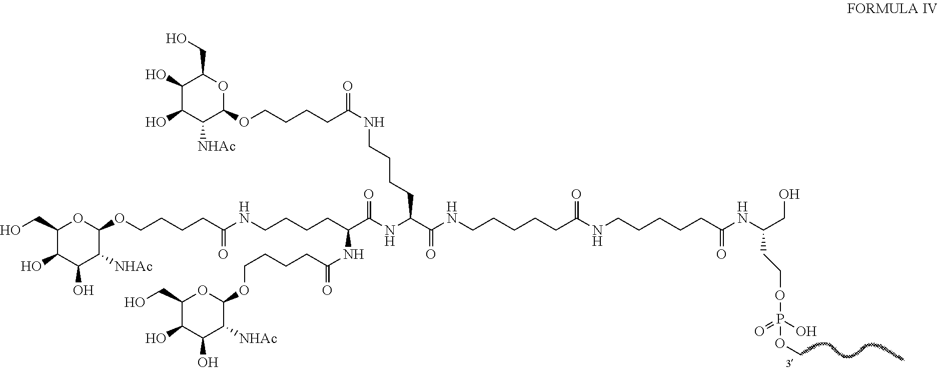 [SMILES: COP(=O)(O)OCC[C@@H](CO)NC(=O)CCCCCNC(=O)CCCCCNC(=O)[C@H](CCCCNC(=O)CCCCO[C@@H]1O[C@H](CO)[C@H](O)[C@H](O)[C@H]1NC(C)=O)NC(=O)[C@H](CCCCNC(=O)CCCCO[C@@H]1O[C@H](CO)[C@H](O)[C@H](O)[C@H]1NC(C)=O)NC(=O)CCCCO[C@@H]1O[C@H](CO)[C@H](O)[C@H](O)[C@H]1NC(C)=O]

In certain embodiments, the oligonucleotide compound comprises a ligand and linker having the following structure of Formula V, wherein each n is independently 1 to 3, k is 1 to 3, and the ligand is attached to the 5′ end of an oligonucleotide of the oligonucleotide compound (e.g. sense strand in a double-stranded oligonucleotide compound) (represented by the solid wavy line):

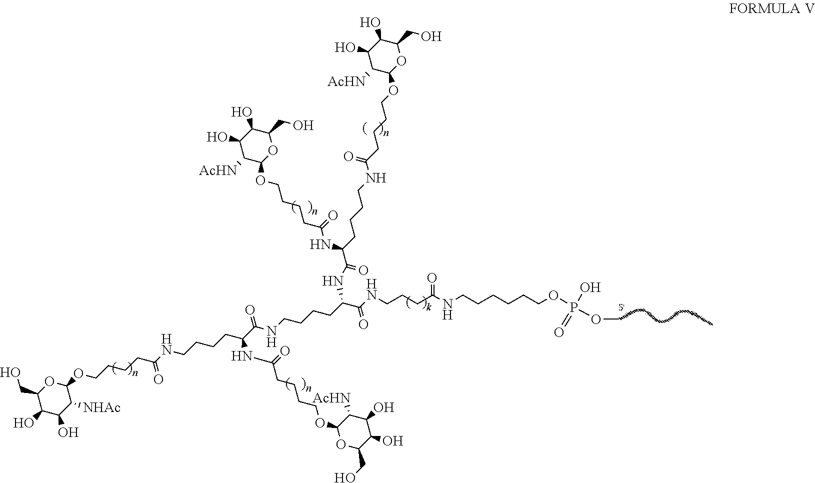

In other embodiments, the oligonucleotide compound comprises a ligand and linker having the following structure of Formula VI, wherein each n is independently 1 to 3, k is 1 to 3, and the ligand is attached to the 5′ end of an oligonucleotide of the oligonucleotide compound (e.g. sense strand in a double-stranded oligonucleotide compound) (represented by the solid wavy line):

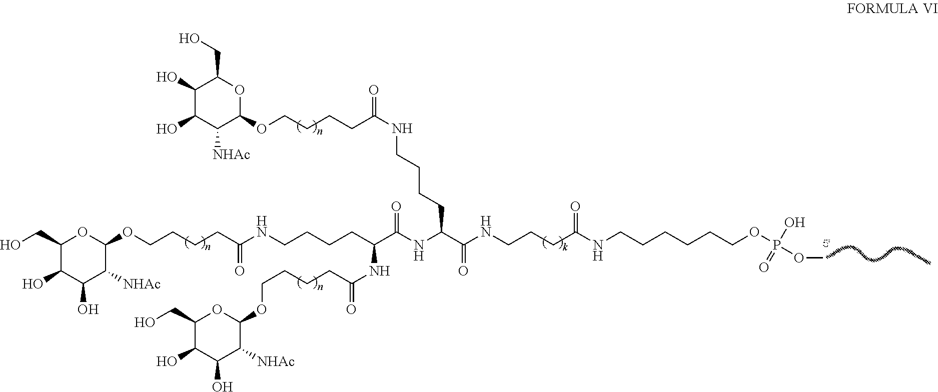

In one particular embodiment, the oligonucleotide compound comprises a ligand and linker having the following structure of Formula VII, wherein X═O or S and wherein the ligand is attached to the 5′ end of an oligonucleotide of the oligonucleotide compound (e.g. sense strand in a double-stranded oligonucleotide compound) (represented by the squiggly line):

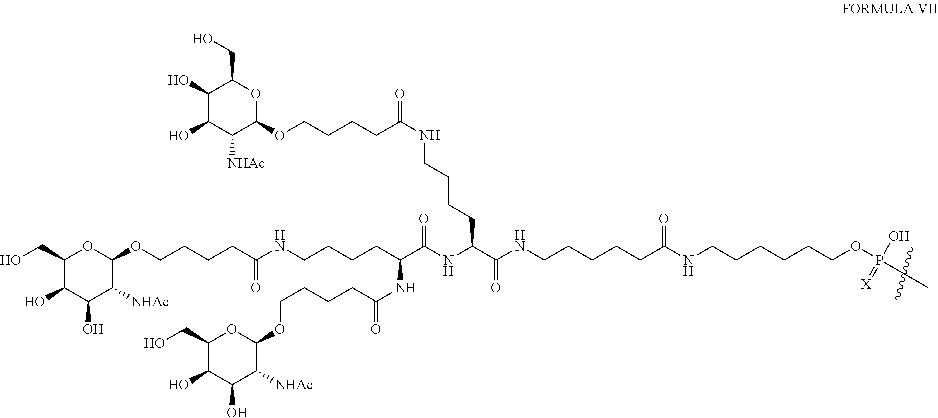

In some embodiments, the oligonucleotide compound comprises a ligand and linker having the following structure of Formula VIII, wherein each n is independently 1 to 3 and the ligand is attached to the 5′ end of an oligonucleotide of the oligonucleotide compound (e.g. sense strand in a double-stranded oligonucleotide compound) (represented by the solid wavy line):

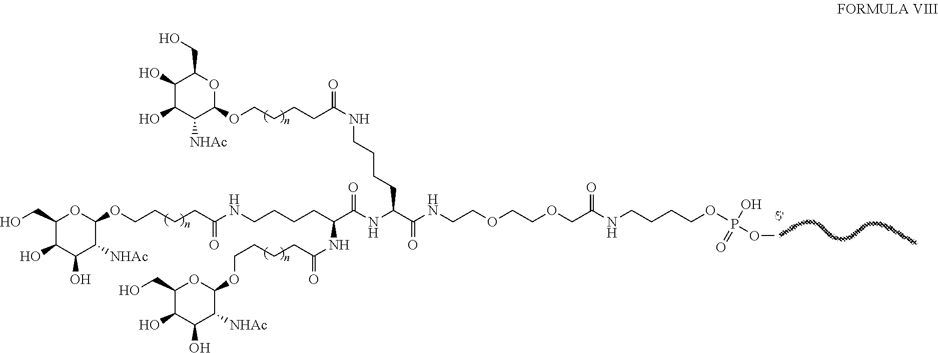

In certain embodiments, the oligonucleotide compound comprises a ligand and linker having the following structure of Formula IX, wherein the ligand is attached to the 5′ end of an oligonucleotide of the oligonucleotide compound (e.g. sense strand in a double-stranded oligonucleotide compound) (represented by the solid wavy line):

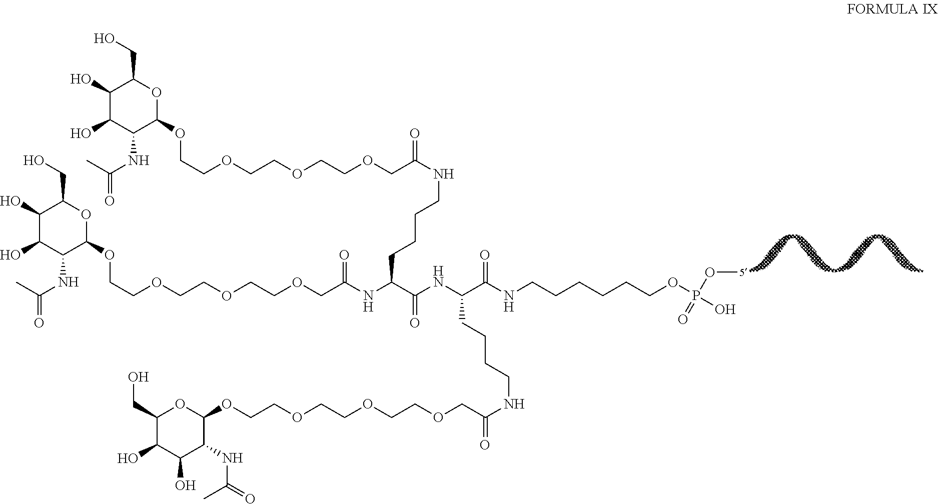 [SMILES: COP(=O)(O)OCCCCCCNC(=O)[C@H](CCCCNC(=O)COCCOCCOCCO[C@@H]1O[C@H](CO)[C@H](O)[C@H](O)[C@H]1NC(C)=O)NC(=O)[C@H](CCCCNC(=O)COCCOCCOCCO[C@@H]1O[C@H](CO)[C@H](O)[C@H](O)[C@H]1NC(C)=O)NC(=O)COCCOCCOCCO[C@@H]1O[C@H](CO)[C@H](O)[C@H](O)[C@H]1NC(C)=O]

A phosphorothioate bond can be substituted for the phosphodiester bond shown in any one of Formulas I-IX to covalently attach the ligand and linker to the oligonucleotide.

The methods of the invention comprise inhibiting the expression or activity of a suppressor protein in a cell. As used herein, a suppressor protein refers to a protein the presence or activity of which reduces or prevents gene silencing activity of an oligonucleotide compound. In certain embodiments, a suppressor protein negatively regulates vesicle trafficking, particularly endosome trafficking, within a cell, thereby reducing or preventing gene silencing activity of ligand-conjugated oligonucleotide compounds that are internalized by receptor-mediated endocytosis. In some embodiments of the methods of the invention, the suppressor protein is Ras-related protein Rab-18 (RAB18), Zw10 kinetochore protein (ZW10), syntaxin 18 (STX18), Sec1 family domain-containing protein 2 (SCFD2), NSF attachment protein gamma (NAPG), sterile alpha motif domain-containing protein 4B (SAMD4B), vacuolar protein sorting-associated protein 37A (VPS37A), yes associated protein 1 (YAP1), cyclin E1 (CCNE1), solute carrier family 30 member 9 protein (SLC30A9), tubulin epsilon and delta complex protein 1 (TEDC1; also known as C14orf80), hypoxia-inducible factor 1-alpha inhibitor protein (HIF1AN), or TNF receptor associated factor 2 (TRAF2). In certain embodiments, the suppressor protein is RAB18, ZW10, STX18, SCFD2, NAPG, SAMD4B, or VPS37A. In certain other embodiments, the suppressor protein is RAB18, ZW10, or STX18. In one particular embodiment of the methods of the invention, the suppressor protein is RAB18.

The expression or activity of a suppressor protein can be inhibited by contacting a cell with an inhibitor of the suppressor protein. An inhibitor of the suppressor protein reduces the intracellular amount or activity of the suppressor protein by at least 30%, at least 35%, at least 40%, at least 45%, at least 50%, at least 55%, at least 60%, at least 65%, at least 70%, at least 75%, at least 80%, at least 85%, at least 90%, or at least 95% relative to the intracellular amount or activity of the suppressor protein in a cell not contacted with the inhibitor. In certain embodiments, the inhibitor of the suppressor protein reduces the intracellular amount or activity of the suppressor protein by about 75, 76, 77, 78, 79, 80, 81, 82, 83, 84, 85, 86, 87, 88, 89, 90, 91, 92, 93, 94, 95, 96, 97, 98, 99, or 100% relative to the intracellular amount or activity of the suppressor protein in a cell not contacted with the inhibitor. In one embodiment, the suppressor protein reduces the intracellular amount or activity of the suppressor protein by at least 75% relative to the intracellular amount or activity of the suppressor protein in a cell not contacted with the inhibitor. In another embodiment, the suppressor protein reduces the intracellular amount or activity of the suppressor protein by at least 80% relative to the intracellular amount or activity of the suppressor protein in a cell not contacted with the inhibitor. In another embodiment, the suppressor protein reduces the intracellular amount or activity of the suppressor protein by at least 90% relative to the intracellular amount or activity of the suppressor protein in a cell not contacted with the inhibitor.

In certain embodiments of the methods of the invention, inhibitors of the suppressor protein may be oligonucleotide-based inhibitors that reduce the expression of nucleic acids (e.g. mRNA) encoding the suppressor protein. For instance, in some embodiments, the inhibitor of the suppressor protein is an oligonucleotide compound as described herein, wherein the oligonucleotide compound comprises a sequence that is substantially or fully complementary to an mRNA sequence encoding the suppressor protein. In some such embodiments, the oligonucleotide compound may be a single-stranded antisense oligonucleotide comprising a sequence that is substantially or fully complementary to an mRNA sequence encoding the suppressor protein. In other embodiments, the inhibitor of the suppressor protein is a double-stranded oligonucleotide compound, such as an siRNA or shRNA as described herein. In one embodiment, the double-stranded oligonucleotide compound is an siRNA molecule comprising a sense strand and an antisense strand, wherein the antisense strand comprises a sequence that is substantially or fully complementary to an mRNA sequence encoding the suppressor protein. In another embodiment, the double-stranded oligonucleotide compound is an shRNA molecule comprising a sense strand and an antisense strand linked together by a loop region, wherein the antisense strand comprises a sequence that is substantially or fully complementary to an mRNA sequence encoding the suppressor protein.

An mRNA sequence encoding the suppressor protein can be any messenger RNA sequence, including allelic variants and splice variants, encoding the suppressor protein, including variants or isoforms of the suppressor protein from any species (e.g. non-human primate, human). An mRNA sequence encoding the suppressor protein also includes the transcript sequence expressed as its complementary DNA (cDNA) sequence. A cDNA sequence refers to the sequence of an mRNA transcript expressed as DNA bases (e.g. guanine, adenine, thymine, and cytosine) rather than RNA bases (e.g. guanine, adenine, uracil, and cytosine). Thus, in some embodiments, the inhibitors of the suppressor protein can be oligonucleotide compounds comprising a region having a sequence that is substantially or fully complementary to a mRNA sequence or cDNA sequence encoding the suppressor protein. References in the Ensembl Genome or National Center for Biotechnology Information (NCBI) databases for exemplary mRNA and cDNA sequences for select suppressor proteins to which the oligonucleotide compound may be substantially or fully complementary are listed below in Table 1.

TABLE 1
Exemplary transcript sequences encoding select suppressor proteins

| Suppressor | |
|---|---|
| Protein | Transcript sequences (mRNA/cDNA) |
| RAB18 | NCBI Reference sequence: NM_021252.5 |
| | Ensembl transcript no. ENST00000356940.11 |
| ZW10 | NCBI Reference sequence: NM_004724.4 |
| | Ensembl transcript no. ENST00000200135.8 |
| STX18 | NCBI Reference sequence: NM_016930.4 |
| | Ensembl transcript no. ENST00000306200.7 |
| SCFD2 | NCBI Reference sequence: NM_152540.4 |
| | Ensembl transcript no. ENST00000401642.8 |
| NAPG | NCBI Reference sequence: NM_003826.3 |
| | Ensembl transcript no. ENST00000322897.11 |
| SAMD4B | NCBI Reference sequence: NM_018028.4 |
| | Ensembl transcript no. ENST00000610417.4 |
| VPS37A | NCBI Reference sequence: NM_152415.3 |
| | Ensembl transcript no. ENST00000324849.9 |

In some embodiments of the methods of the invention, the suppressor protein is RAB18 and the inhibitor of RAB18 is a single-stranded antisense oligonucleotide comprising or consisting of a nucleotide sequence selected from: SEQ ID NOs: 8 to 10, 5′-UUUAGCCUUAUUUCCAUCC-3′ (SEQ ID NO: 25), 5′-AACGUAUCAUCUGUGAACC-3′ (SEQ ID NO: 26), 5′-AUCGACUUCACGAUUUUCC-3′ (SEQ ID NO: 27), 5′-CCUCUAUAAUAGCUGGGAGUUA-3′ (SEQ ID NO: 28), and 5′-CCCUGUGCACCUCUAUAAUAGC-3′ (SEQ ID NO: 29). In certain embodiments, the inhibitor of RAB18 is a single-stranded antisense oligonucleotide comprising or consisting of a nucleotide sequence of any one of SEQ ID NOs: 25 to 29 where thymine is substituted for uracil. In other embodiments, the suppressor protein is RAB18 and the inhibitor of RAB18 is an siRNA comprising a sense strand and an antisense strand, wherein the antisense strand comprises or consists of a nucleotide sequence selected from SEQ ID NOs: 8 to 10 and 25 to 29. In such embodiments, the nucleotide sequence of the sense strand is substantially or fully complementary to the sequence of the antisense strand. In certain embodiments, the inhibitor of RAB18 is an siRNA comprising a sense strand and an antisense strand, wherein: (i) the sense strand comprises or consists of the nucleotide sequence of SEQ ID NO: 5 and the antisense strand comprises or consists of the nucleotide sequence of SEQ ID NO: 8; (ii) the sense strand comprises or consists of the nucleotide sequence of SEQ ID NO: 6 and the antisense strand comprises or consists of the nucleotide sequence of SEQ ID NO: 9; or (iii) the sense strand comprises or consists of the nucleotide sequence of SEQ ID NO: 7 and the antisense strand comprises or consists of the nucleotide sequence of SEQ ID NO: 10.

In some embodiments of the methods of the invention, inhibiting the expression or activity of the suppressor protein in a cell can include modifying the gene encoding the suppressor protein using any suitable known genome editing technique, including but not limited to CRISPR-Cas-based methods, transcription activator-like effector nuclease (TALEN)-based methods, and zinc finger nuclease (ZFN)-based methods (see, e.g., Porteus, Annual Review of Pharmacology and Toxicology, Vol. 56:163-190, 2016; Maeder and Gersbach, Mol Ther., Vol. 24:430-446, 2016). The gene encoding the suppressor protein may be modified such that the gene encodes a variant of the suppressor protein that has reduced activity or function, or the gene may be modified to eliminate expression of the gene entirely (i.e. knock out the gene). Thus, in such embodiments, the inhibitor of the suppressor protein may be a gene modifying agent. Depending on the genome editing technique employed, the gene modifying agent may comprise a nuclease (e.g. Cas nuclease, TALEN, or ZFN) or a vector encoding the nuclease and/or a guide RNA. A guide RNA refers to a polynucleotide comprising a sequence having sufficient complementarity with a target nucleic acid sequence to hybridize to the target sequence and direct sequence-specific binding of a Cas nuclease to the target sequence. For TALEN-based or ZFN-based methods, the nuclease is engineered to recognize a portion of the gene sequence encoding the suppressor protein, such as any of the sequences set forth in Table 1. In embodiments in which a CRISPR-Cas system is used to modify the gene encoding the suppressor protein, the guide RNA comprises a sequence that is complementary to a portion of the gene sequence encoding the suppressor protein, such as any of the sequences set forth in Table 1. Methods of designing guide RNAs to modify a target gene sequence are known to those of skill in the art, such as the methods described in Mohr et al., The FEBS Journal, Vol. 283:3232-3238, 2016 and Brazelton et al., GM Crops & Food, Vol. 6:266-276, 2015. In certain embodiments, the inhibitor of the suppressor protein is a gene modifying agent comprising a Cas nuclease or vector/nucleic acid encoding the Cas nuclease and a guide RNA comprising a sequence that is complementary to a portion of the gene sequence encoding the suppressor protein. As described further herein, the gene modifying agent can be delivered into the cell using viral vectors encoding the nuclease or both the Cas nuclease and the guide RNA in the case of CRISPR-Cas system use as well as by lipid-based delivery vehicles in which the nuclease or a Cas nuclease-guide RNA complex can be packaged.

In some embodiments of the methods of the invention, the suppressor protein is RAB18 and the inhibitor of RAB18 is a gene modifying agent comprising a guide RNA having a sequence that is complementary to the sequence of SEQ ID NO: 11 or SEQ ID NO: 12. In related embodiments, the inhibitor of RAB18 is a gene modifying agent comprising a guide RNA comprising a sequence selected from SEQ ID NOs: 8 to 10 and 25 to 29. In any of the foregoing embodiments, the gene modifying agent may further comprise a Cas nuclease or a vector/nucleic acid encoding a Cas nuclease.

In certain embodiments, the present invention provides methods for enhancing the silencing activity of an oligonucleotide compound in a cell comprising inhibiting the expression or activity of a suppressor protein in the cell, for example, by contacting the cell with an inhibitor of the suppressor protein, such as any of the inhibitors described herein or known in the art, and contacting the cell with the oligonucleotide compound. Enhancing the silencing activity of an oligonucleotide compound, such as any of the oligonucleotide compounds described herein, means that the silencing activity in terms of level of reduction, duration of reduction, and/or potency of reduction in gene expression is increased in a cell relative to the silencing activity of the oligonucleotide compound in a cell in which the expression or activity of the suppressor protein is not inhibited or in a cell not contacted with an inhibitor of the suppressor protein. The silencing activity of an oligonucleotide compound may be enhanced by the methods of the invention by at least 2-fold, at least 4-fold, at least 8-fold, at least 10-fold, at least 15-fold, at least 20-fold, at least 25-fold, or at least 30-fold as compared to the silencing activity of the oligonucleotide compound in a cell in which the expression or activity of the suppressor protein is not inhibited or in a cell not contacted with an inhibitor of the suppressor protein. Silencing activity of an oligonucleotide compound can be assessed by measuring the amount of expression of the target gene in the presence of the oligonucleotide compound as compared to the amount of target gene expression in a cell in the absence of the oligonucleotide compound. Target gene expression can be assessed by measuring the amount or level of target mRNA, target protein, or another biomarker linked to expression of the target gene as further described below.

As described in the Examples herein, inhibition or elimination of the expression of suppressor proteins (e.g. RAB18) significantly increased the level of knockdown of a target gene mediated by a ligand-conjugated oligonucleotide compound, thereby making it possible to reduce expression of even highly abundant proteins, which are normally unable to be silenced using oligonucleotide compounds. Thus, the present invention also includes methods of reducing expression of a target gene in a cell comprising contacting the cell with an inhibitor of a suppressor protein as described herein and contacting the cell with an oligonucleotide compound, such as the oligonucleotide compounds described herein, wherein the oligonucleotide compound comprises a sequence that is substantially or fully complementary to a sequence of the target gene. The cell may be in vitro or in vivo. In some embodiments, the cell is in a subject (e.g. a human subject) in need of reduced expression of the target gene. The cell may be a cell that natively expresses the target gene or a cell or cell line that has been engineered to express the target gene. In some embodiments, the cell is a mammalian cell or cell line. The cell may be a cell from any tissue type that expresses the target gene including, but not limited to, an adipocyte, an epithelial cell, a neuron, a glial cell, a cardiomyocyte, a skeletal myocyte, a pancreatic beta cell, a macrophage, a B cell, a tumor cell, or a hepatocyte. In certain embodiments, the cell is a hepatocyte, such as a primary hepatocyte. In other embodiments, the cell is liver cell line, such as HepAD38 cells, HuH-6 cells, HuH-7 cells, HuH-5-2 cells, BNLCL2 cells, Hep3B cells, or HepG2 cells. In one embodiment, the cell is a Hep3B cell.

The reduction of target gene expression in cells or animals contacted with an oligonucleotide compound according to the methods of the invention can be determined relative to the target gene expression in cells or animals not contacted with the oligonucleotide compound or contacted with a control oligonucleotide compound. For instance, in some embodiments, reduction of target gene expression is assessed by (a) measuring the amount of level of target mRNA in cells contacted with an oligonucleotide compound according to the methods of the invention, (b) measuring the amount or level of target mRNA in cells contacted with a control oligonucleotide compound (e.g. an oligonucleotide compound directed to a RNA molecule not expressed in the cells or an oligonucleotide compound having a nonsense or scrambled sequence) or no compound, and (c) comparing the measured target mRNA levels from treated cells in (a) to the measured target mRNA levels from control cells in (b). The target mRNA levels in the treated cells and controls cells can be normalized to RNA levels for a control gene (e.g. 18S ribosomal RNA or housekeeping gene) prior to comparison. Target mRNA levels can be measured by a variety of methods, including Northern blot analysis, nuclease protection assays, fluorescence in situ hybridization (FISH), reverse-transcriptase (RT)-PCR, real-time RT-PCR, quantitative PCR, droplet digital PCR, and the like.

In other embodiments, reduction of target gene expression is assessed by (a) measuring the amount or level of target protein in cells contacted with an oligonucleotide compound according to the methods of the invention, (b) measuring the amount or level of target protein in cells contacted with a control oligonucleotide compound (e.g. an oligonucleotide compound directed to a RNA molecule not expressed in the cells or an oligonucleotide compound having a nonsense or scrambled sequence) or no compound, and (c) comparing the measured target protein levels from treated cells in (a) to the measured target protein levels from control cells in (b). Methods of measuring target protein levels are known to those of skill in the art, and include Western Blots, immunoassays (e.g. ELISA), and flow cytometry.

The present invention also provides methods for reducing expression of a target gene in a subject in need thereof comprising administering to the subject: an inhibitor of a suppressor protein as described herein and an oligonucleotide compound, such as the oligonucleotide compounds described herein, wherein the oligonucleotide compound comprises a sequence that is substantially or fully complementary to a sequence of the target gene. In some embodiments of the methods of the invention, expression of the target gene is associated with a disease or disorder, for example, where overexpression of a gene product or expression of a protein variant or isoform (e.g. mutant form of the gene product) causes a pathological phenotype. In certain embodiments of the methods of the invention, the target gene is a human gene. Exemplary target genes include, but are not limited to, LPA, PNPLA3, ASGR1, F7, F12, FXI, APOCIII, APOB, APOL1, TTR, PCSK9, HSD17B13, HPRT1, PPIB, EPAS1, DUX4, DMPK, XDH, SCNN1A, SCAP, KRAS, CD274, PDCD1, C3, C5, CFB, ALAS1, GYS1, HAO1, LDHA, ANGPTL3, SERPINA1, ALDH2, AGT, HAMP, LECT2, EGFR, VEGF, KIF11, AT3, CTNNB1, HMGB1, HIF1A, and STAT3. Target genes may also include viral genes, such as hepatitis B and hepatitis C viral genes, human immunodeficiency viral genes, herpes viral genes, etc. In some embodiments of the methods of the invention, the target gene is a gene that encodes a human micro RNA (miRNA). In certain embodiments of the methods of the invention, the target gene is a gene expressed in the liver.

In some embodiments of the methods of the invention, expression of the target gene is reduced in cells or a subject by at least 50%. In other embodiments of the methods of the invention, expression of the target gene is reduced in cells or a subject by at least 60%, at least 65%, at least 70%, at least 75%, at least 80%, or at least 85%. In still other embodiments, the expression of a target gene is reduced in cells or a subject by about 90% or more, e.g., 91%, 92%, 93%, 94%, 95%, 96%, 97%, 98%, 99%, or more. The percent reduction of target gene expression can be measured by any of the methods described herein as well as others known in the art.

In certain embodiments of the methods of the invention, the methods comprise contacting a cell with an inhibitor of a suppressor protein and a first oligonucleotide compound comprising a sequence that is substantially or fully complementary to the sequence of a target gene, wherein the first oligonucleotide compound is covalently attached to a ligand of a receptor expressed on the surface of the cell, and wherein the inhibitor of a suppressor protein is a second oligonucleotide compound comprising a sequence that is substantially complementary or fully complementary to a mRNA sequence encoding the suppressor protein. In some embodiments, the first oligonucleotide compound is a single-stranded antisense oligonucleotide. In other embodiments, the first oligonucleotide compound is an siRNA. In any of the foregoing embodiments, the second oligonucleotide compound is a single-stranded antisense oligonucleotide or an siRNA.

In certain other embodiments of the methods of the invention, the methods comprise administering to a subject an inhibitor of a suppressor protein and a first oligonucleotide compound comprising a sequence that is substantially or fully complementary to the sequence of a target gene, wherein the first oligonucleotide compound is covalently attached to a first ligand, and wherein the inhibitor of a suppressor protein is a second oligonucleotide compound comprising a sequence that is substantially complementary or fully complementary to a mRNA sequence encoding the suppressor protein. In some embodiments, the first oligonucleotide compound is a single-stranded antisense oligonucleotide. In other embodiments, the first oligonucleotide compound is an siRNA. In any of the foregoing embodiments, the second oligonucleotide compound is a single-stranded antisense oligonucleotide or an siRNA. In some embodiments, the second oligonucleotide compound is covalently attached to a second ligand. The first ligand, the second ligand, or both the first and second ligands may be any of the ligands described herein. For instance, in some embodiments, the first ligand, the second ligand, or both the first and second ligands comprise a cholesterol moiety, a vitamin, a steroid, a bile acid, a folate moiety, a fatty acid, a carbohydrate, a glycoside, or antibody or antigen-binding fragment thereof. In other embodiments, the first ligand, the second ligand, or both the first and second ligands comprise galactose, galactosamine, or N-acetyl-galactosamine. In one embodiment, the first ligand, the second ligand, or both the first and second ligands comprise a multivalent galactose moiety or multivalent N-acetyl-galactosamine moiety. In any of the foregoing embodiments, the first ligand covalently attached to the first oligonucleotide compound is the same as the second ligand covalently attached to the second oligonucleotide compound. In some such embodiments, the first and second ligands are ligands of a receptor expressed on the surface of a liver cell, such as the ASGPR. In other embodiments of the methods of the invention, the first ligand is different than the second ligand, but both the first and second ligands are ligands of a receptor(s) expressed on the same cell type. By way of example, the first ligand may comprise a multivalent N-acetyl-galactosamine moiety, which is a ligand of ASGPR expressed in hepatocytes, and the second ligand may comprise a cholesterol moiety, a ligand of LDL receptor also expressed in hepatocytes.

The oligonucleotide compounds and gene modifying agents described herein can be delivered into a cell by various methods including by transfection, viral transduction, lipid-based particles, and conjugation to ligands as further described herein. In some embodiments the oligonucleotide compounds and/or gene modifying agents are expressed from a vector. A “vector” refers to any molecule or entity (e.g., nucleic acid, plasmid, bacteriophage or virus) used to transfer genetic material into a host cell. Examples of vectors include, but are not limited to, plasmids, viral vectors, non-episomal mammalian vectors and expression vectors, for example, recombinant expression vectors. Suitable viral vectors, which are preferred in some embodiments, include adenoviral vectors, adeno-associated viral vectors, and retroviral vectors, such as lentiviral vectors. The term “expression vector” or “expression construct” as used herein refers to a recombinant nucleic acid molecule containing a desired target sequence and appropriate nucleic acid control sequences necessary for the expression of the operably linked target sequence in a particular cell. An expression vector can include, but is not limited to, sequences that affect or control transcription, translation, and, if introns are present, affect RNA splicing of a coding region operably linked thereto. Nucleic acid sequences necessary for expression in eukaryotic cells include a promoter, optionally an enhancer sequence, and termination and polyadenylation signals.

In some embodiments in which the inhibitor of a suppressor protein is an oligonucleotide compound as described herein, the inhibitor of the suppressor protein is delivered into a cell using a vector (e.g. viral vector) comprising the oligonucleotide operably linked to a promoter (e.g. RNA pol III promoter) such that the oligonucleotide compound is expressed in the cell. In such embodiments, the oligonucleotide sequence operably linked to the promoter may be an antisense oligonucleotide or shRNA as described above. The term “operably linked” as used herein refers to the linkage of two or more nucleic acid sequences in such a manner that a nucleic acid molecule capable of directing the transcription of a given gene and/or the synthesis of a desired protein molecule is produced. For example, a control sequence in a vector that is “operably linked” to a nucleotide sequence is ligated thereto so that expression of the nucleotide sequence is achieved under conditions compatible with the transcriptional activity of the control sequences. A large number of promoters, recognized by a variety of potential host cells, are well known to those of skill in the art. For example, suitable promoters for use with mammalian host cells include those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus, and Simian Virus 40 (SV40). In embodiments in which the desired product is an oligonucleotide compound or guide RNA, the promoter may be an RNA pol III promoter, such as a U6 promoter.

In other embodiments in which the inhibitor of a suppressor protein is a gene modifying agent comprising a nuclease, such as a Cas nuclease, ZFN, or TALEN, the nuclease can be delivered to the cell using a vector (e.g. viral vector) comprising a nucleotide sequence encoding the nuclease operably linked to a suitable promoter for expression in the cell of interest. In embodiments in which the gene modifying agent further comprises a guide RNA (e.g. when a CRISPR-Cas genome editing method is employed), the vector may further comprise a guide RNA expression cassette comprising the guide RNA sequence operably linked to an RNA pol III promoter, such a U6 promoter. In alternative embodiments, the cell can be contacted with a second vector comprising the guide RNA expression cassette simultaneously or after contact with the first vector encoding the nuclease.

In other embodiments of the methods of the invention, the oligonucleotide compounds and gene modifying agents described herein can be delivered into a cell using lipid-based delivery methods. For example, colloidal dispersion systems, such as macromolecule complexes, nanocapsules, microspheres, beads, and lipid-based systems, including oil-in-water emulsions, micelles, mixed micelles, and liposomes, may be used as delivery vehicles for the oligonucleotide compounds and gene modifying agents. Commercially available fat emulsions that are suitable for delivering nucleic acids include Intralipid® (Baxter International Inc.), Liposyn® (Abbott Pharmaceuticals), Liposyn®II (Hospira), Liposyn®III (Hospira), Nutrilipid (B. Braun Medical Inc.), and other similar lipid emulsions. A preferred colloidal system for use as a delivery vehicle in vivo is a liposome (i.e., an artificial membrane vesicle). The oligonucleotide compounds and/or gene modifying agents may be encapsulated within liposomes or may form complexes thereto, in particular to cationic liposomes. Alternatively, oligonucleotide compounds and/or gene modifying agents may be complexed to lipids, in particular to cationic lipids. Suitable lipids and liposomes include neutral (e.g., dioleoylphosphatidyl ethanolamine (DOPE), dimyristoylphosphatidyl choline (DMPC), and dipalmitoyl phosphatidylcholine (DPPC)), distearolyphosphatidyl choline), negative (e.g., dimyristoylphosphatidyl glycerol (DMPG)), and cationic (e.g., dioleoyltetramethylaminopropyl (DOTAP) and dioleoylphosphatidyl ethanolamine (DOTMA)). The preparation and use of such colloidal dispersion systems are well known in the art. Exemplary formulations are also disclosed in U.S. Pat. Nos. 5,981,505; 6,217,900; 6,383,512; 5,783,565; 7,202,227; 6,379,965; 6,127,170; 5,837,533; 6,747,014; and WIPO Publication No. WO 03/093449.

In some embodiments, the oligonucleotide compounds and/or gene modifying agents are fully encapsulated in a lipid formulation, e.g., to form a SNALP or other nucleic acid-lipid particle. As used herein, the term “SNALP” refers to a stable nucleic acid-lipid particle SNALPs typically contain a cationic lipid, a non-cationic lipid, and a lipid that prevents aggregation of the particle (e.g., a PEG-lipid conjugate). SNALPs are exceptionally useful for systemic applications, as they exhibit extended circulation lifetimes following intravenous injection and accumulate at distal sites (e.g., sites physically separated from the administration site). The nucleic acid-lipid particles typically have a mean diameter of about 50 nm to about 150 nm, about 60 nm to about 130 nm, about 70 nm to about 110 nm, or about 70 nm to about 90 nm, and are substantially nontoxic. In addition, the nucleic acids when present in the nucleic acid-lipid particles are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; and WIPO Publication No. WO 96/40964. Thus, in some embodiments in which the inhibitor of a suppressor protein is an oligonucleotide compound as described herein, the oligonucleotide compound may be encapsulated into a SNALP or other type of liposome. Similarly, in some embodiments in which the inhibitor of a suppressor protein is a gene modifying agent comprising a nuclease (e.g. Cas nuclease, ZFN, or TALEN), a mRNA encoding the nuclease may be incorporated into a SNALP or other liposome optionally with a guide RNA (e.g., when a CRISPR-Cas system is used).

In certain preferred embodiments of the methods of the invention, the oligonucleotide compounds targeting a gene of interest are delivered to a cell in vitro or in vivo by conjugation to a ligand of a receptor expressed on the surface of the cell as described in detail above. Thus, in such embodiments, the oligonucleotide compound may be formulated in a pharmaceutical composition comprising one or more pharmaceutically acceptable excipients. Such compositions are useful for reducing expression of a target gene in a subject in need thereof. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

The phrases “pharmaceutically acceptable” or “pharmacologically acceptable” refer to molecular entities and compositions that do not produce adverse, allergic, or other untoward reactions when administered to an animal or a human. As used herein, “pharmaceutically acceptable excipient” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the oligonucleotide compounds, its use in therapeutic compositions is contemplated. Supplementary active ingredients also can be incorporated into the compositions, provided they do not inactivate the oligonucleotide compounds of the compositions.

Compositions and methods for the formulation of pharmaceutical compositions depend on a number of criteria, including, but not limited to, route of administration, type and extent of disease or disorder to be treated, or dose to be administered. In some embodiments, the pharmaceutical compositions are formulated based on the intended route of delivery. For instance, in certain embodiments, the pharmaceutical compositions are formulated for parenteral delivery. Parenteral forms of delivery include intravenous, intraarterial, subcutaneous, intrathecal, intraperitoneal or intramuscular injection or infusion. In one embodiment, the pharmaceutical composition is formulated for intravenous delivery. In another embodiment, the pharmaceutical composition is formulated for subcutaneous delivery.

In some embodiments, the pharmaceutical compositions comprise an effective amount of an oligonucleotide compound described herein. An “effective amount” is an amount sufficient to produce a beneficial or desired clinical result. In some embodiments, an effective amount is an amount sufficient to reduce target gene expression in a particular tissue or cell-type (e.g. liver or hepatocytes) of a subject.

Administration of the pharmaceutical compositions may be via any common route so long as the target tissue is available via that route. Such routes include, but are not limited to, parenteral (e.g., subcutaneous, intramuscular, intraperitoneal or intravenous), oral, nasal, buccal, intradermal, transdermal, and sublingual routes, or by direct injection into the target tissue (e.g. liver tissue) or delivery through the hepatic portal vein. In some embodiments, the pharmaceutical composition is administered parenterally. For instance, in certain embodiments, the pharmaceutical composition is administered intravenously. In other embodiments, the pharmaceutical composition is administered subcutaneously.

The pharmaceutical compositions suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present invention generally may be formulated in a neutral or salt form. Pharmaceutically-acceptable salts include, for example, acid addition salts (formed with free amino groups) derived from inorganic acids (e.g., hydrochloric or phosphoric acids), or from organic acids (e.g., acetic, oxalic, tartaric, mandelic, and the like). Salts formed with the free carboxyl groups can also be derived from inorganic bases (e.g., sodium, potassium, ammonium, calcium, or ferric hydroxides) or from organic bases (e.g., isopropylamine, trimethylamine, histidine, procaine and the like).

For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). For human administration, preparations should meet sterility, pyrogenicity, general safety and purity standards as required by FDA standards. In certain embodiments, a pharmaceutical composition comprises or consists of a sterile saline solution and an oligonucleotide compound described herein. In other embodiments, a pharmaceutical composition of the invention comprises or consists of an oligonucleotide compound described herein and sterile water (e.g. water for injection, WFI). In still other embodiments, a pharmaceutical composition of the invention comprises or consists of an oligonucleotide compound described herein and phosphate-buffered saline (PBS).

The following examples, including the experiments conducted and the results achieved, are provided for illustrative purposes only and are not to be construed as limiting the scope of the appended claims.

### EXAMPLES

### Example 1. Identification of Proteins Regulating siRNA-Mediated Gene Silencing

To reveal cellular factors limiting intracellular delivery of siRNA therapeutic molecules, a pooled, genome wide loss-of-function screen was performed utilizing the delivery of an N-acetylgalactosamine (GalNAc) moiety-conjugated siRNA targeting the HPRT1 gene in the human hepatocellular carcinoma Hep3B cell line. The Hep3B cell line was chosen for the screen because of its proliferative potential and high expression levels of the asialoglycoprotein receptor (ASGPR). In addition, Hep3B cells displayed robust knockdown of target genes through GalNAc moiety-conjugated siRNA-induced silencing (data not shown).

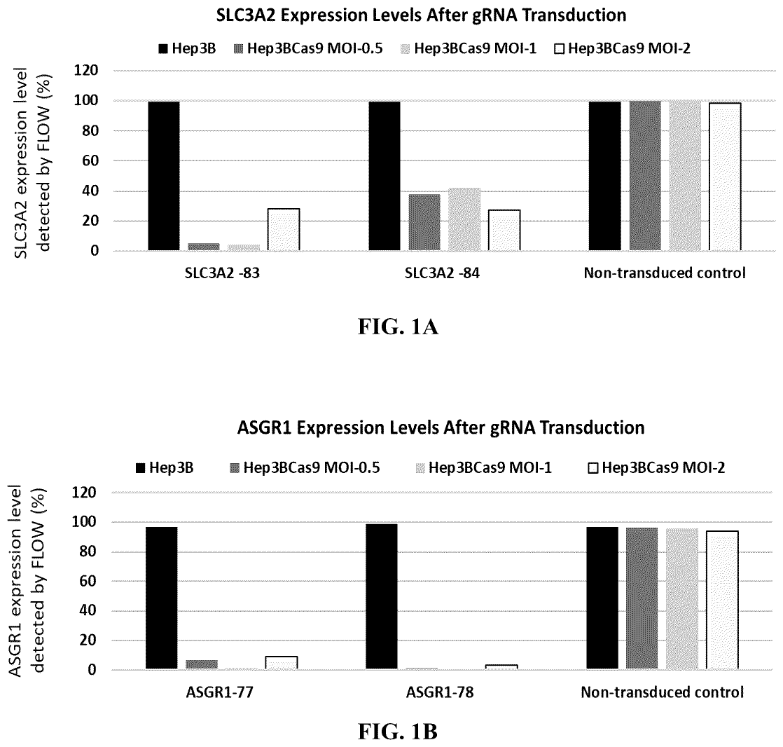

Hep3B cells stably expressing CRISPR-Associated Protein 9 (Cas9) were generated by transducing the cells with a TransEDIT CRISPR Cas9 nuclease expression lentivirus (pCLIP-Cas9-Nuclease-EFS-Blast; TransOMIC technologies, Huntsville, AL, Cat #NC0956087) at three different multiplicities of infection (MOI; 0.5, 1, and 2). All cells were selected and maintained with 10 μg/mL blasticidin after transduction. No toxicities were observed in any of the Cas9 stable expression Hep3B pools. The editing capability of the Cas9 stable Hep3B cells was assayed through validating the editing efficacy on two target genes, SLC3A2 and ASGR1. Two different guide RNA (gRNA) lentivirus vectors targeting the SLC342 gene (SLC3A2-83 and SLC3A2-84) or the ASGR1 gene (ASGR1-77 and ASGR1-78) were transduced individually into both parental Hep3B cell lines and each of the Cas9 stable Hep3B pools. These gRNA lentivirus vectors are described below in Table 2. The SLC3A2 and ASGR1 expression levels before and after gRNA lentivirus transduction were measured through antibody staining followed by flow cytometry analysis. Compared to the parental Hep3B cell line, both target genes were successfully knocked out in all Cas9 stable Hep3B pools (FIGS. 1A and 1B), demonstrating the Cas9 stable Hep3BCas9 cells were fully equipped with editing function. Since the editing effects were similar in all three Cas9 stable Hep3B pools, to minimize potential Cas9 toxicity the one with lowest MOI (0.5, referred to as Hep3BCas9) was chosen to perform the Clustered Regularly Interspaced Short Palindromic Repeats (CRISPR) screen to identify possible regulators of GalNAc moiety-conjugated siRNA-induced silencing.

TABLE 2
Lentiviral gRNA vectors for validating Cas9 editing
function in Hep3BCas9 stable cells

| | Sanger | Target | | SEQ ID |
|---|---|---|---|---|
| gRNA Name | Clone ID | Gene | DNA Target Sequence | NO. |
| SLC3A2-83 | HS5000011883 | SLC3A2 | GCATGACTGGAGCCTCCATAGG | 1 |
| SLC3A2-84 | HS5000011884 | SLC3A2 | CCAGCTTCCCTGACATCCCAGG | 2 |
| ASGR1-77 | HS5000003177 | ASGR1 | TTCACGTGGAGCAGCAGGCTGG | 3 |
| ASGR1-78 | HS5000003178 | ASGR1 | CGGAGCCTGAGCTGTCAGATGG | 4 |

An HPRT1-6TG based live/dead selection system was used in the CRISPR knockout screen to identify potential regulators of GalNAc moiety-conjugated siRNA efficacy. 6-thioguanine (6TG), a purine analog, is incorporated into DNA and RNA after being phosphorylated by hypoxanthine phosphoribosyl transferase (HPRT) encoded in humans by the HPRT1 gene, resulting in cell death (Liao et al., Nucleic Acids Res, Vol. 43 (20): e134, doi: 10.1093/nar/gkv675, 2015). Knocking down or knocking out HPRT1 expression in cells provides resistance to 6TG and allows those cells to survive. A GalNAc moiety-conjugated siRNA targeting human HPRT1 incorporating 2′-fluoro and 2′-methoxy (OMe) modifications (duplex no. 8172) was designed and validated. The sense and antisense sequences for duplex no. 8172 are set forth in Table 5 below. If the GalNAc moiety-conjugated HPRT1 siRNA can enter the cells and induce HPRT1 gene silencing, these cells would be able to survive in the presence of 6TG. Otherwise, the cells would be killed by 6TG selection. Under the CRISPR knockout condition, if a gene is normally required for siRNA activity, knocking out this gene would diminish or abolish siRNA function and cause the cells to be eliminated by 6TG selection. Alternatively, if a gene normally functions to inhibit or block siRNA activity, knocking out this gene would improve siRNA potency and enable the cells to survive 6TG selection. Therefore, when sequencing gRNAs in surviving cells, the enriched gRNAs reflect genes that may normally inhibit siRNA activity, while gRNAs targeting genes essential for siRNA function would be depleted. However, other gRNAs targeting genes that impact cell viability through non-siRNA related mechanisms would also be depleted from live cell population, making it difficult to identify siRNA essential genes from the depleted gRNA population. Therefore, the analysis from the CRISPR knockout screen was focused on the enriched gRNAs from surviving cells to enable the identification of genes that inhibit GalNAc moiety-conjugated siRNA induced silencing.

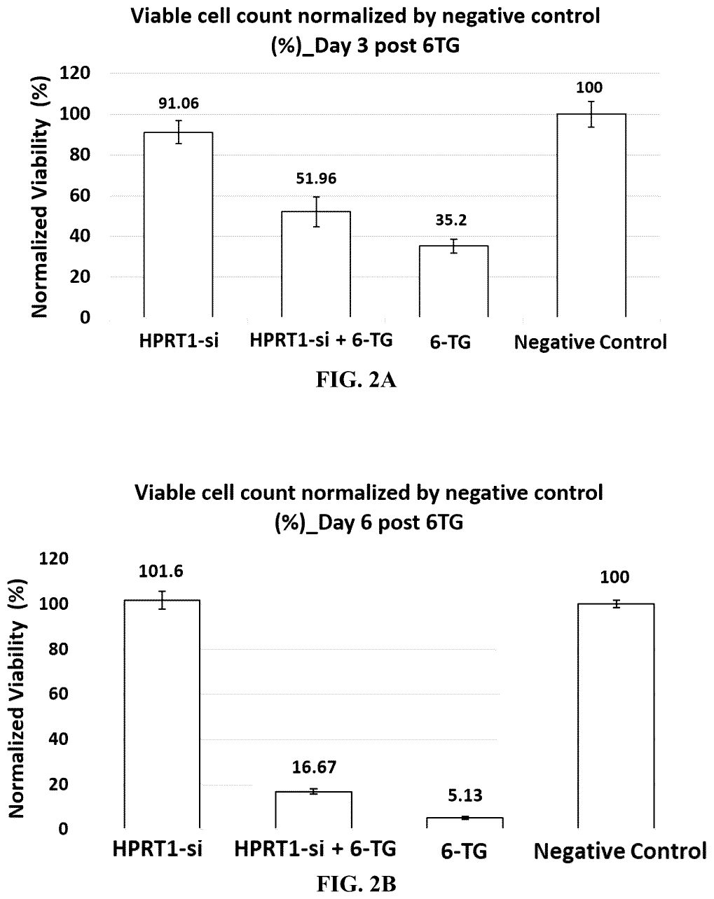

First, the baseline 6TG kill curve in Hep3BCas9 cells without siRNA treatment was established. To avoid both insufficient and excessive killing caused by 6TG, a small-scale pilot run using 100 μM 6TG (˜IC70) and 20 μM 6TG (˜IC50) was performed. Hep3BCas9 cells were transduced with an 80K genome wide gRNA lentivirus library (CRISPR KOHGW 80K (lot #17050301), Cellecta, Mountain View, CA) to generate a genome wide knockout pool. The cells were first equally divided into four groups (0.6E+06 cells/group): 1) siRNA only, 2) siRNA with 6TG treatment, 3) 6TG only, and 4) negative control. To obtain sufficient but not excessive siRNA effect, 750 nM (about IC60) GalNAc moiety-conjugated HPRT1 siRNA (duplex no. 8172) was added to group 1 and 2 on day 0 of experiment. On day 3 of experiment, the tissue culture media was removed from each group and then 100 μM 6TG or 20 μM 6TG was added to group 2 and 3, while non-selection full growth media was added to group 1 and 4. The cells were incubated for 3 days after 6TG treatment. Then cells were split and the 6TG media was replaced with full growth media without 6TG and cultured for additional 3 days. The cell count readings (measured by ViCell) were recorded on day 3 post-6TG treatment and day 6 post-6TG treatment (FIGS. 2A and 2B). As illustrated in FIG. 2A, on day 3 post-6TG treatment, the 6TG only group had 35% viable cells while the HPRT1-si+6TG group had 52% viable cells. On day 6 post-6TG treatment, the 6TG only group had only 5% viable cells while the HPRT1-si+6TG group had 17% viable cells (FIG. 2B). These results indicate that GalNAc moiety conjugated-HPRT1 siRNA treatment was partially protective. This provides a screening phenotype well-suited for detecting gene knockouts that enhance RNA interference (RNAi) activity. Based on the findings from this initial screen, the 6-day 100 μM 6TG treatment was selected as the condition for the large-scale genome wide knockout screen.

To test the impact of siRNA dosage, the large-scale screen was performed with two different concentrations of the GalNAc moiety conjugated-HPRT1 siRNA (duplex no. 8172)-150 nM siRNA conjugate (low dose group) and 750 nM conjugate (high dose group). Hep3BCas9 cells were transduced with a gRNA lentivirus library, which expresses gRNA under a wild-type U6 promoter and TagRFP and Puro resistance genes under a human ubiquitin C promoter. This library covers approximately 19,000 genes with 4 gRNA for each gene. The gRNA lentivirus library was transduced into 9.2E+07 Hep3BCas9 cells. The actual library transduction efficiency as reflected by RFP positive cell population (61%) was checked through flow cytometry analysis on day 4 post-transduction. Based on calculation, the actual gRNA lentivirus library transduction MOI was about 0.9, and the actual coverage was 1035. The transduced cells were then selected with puromycin and blasticidin for 14 days. On day 14 post-selection, 87% of the cells were RFP positive (indicating 87% of the cells had an integrated gRNA) by flow cytometry. On day 14 post-selection, 1E+08 cells were collected and frozen as baseline sample. The rest of cells were equally divided into three groups (2.4E+08 cells/group): group 1 was treated with 150 nM GalNAc moiety conjugated-HPRT1 siRNA (duplex no. 8172) as low dose group, group 2 was treated with 750 nM GalNAc moiety conjugated-HPRT1 siRNA as high dose group, and group 3 was set as no siRNA control. On day 3 post-siRNA treatment, 2E+08 cells were collected and frozen from each group as before 6TG treatment samples, then the rest of the cells in each group were further divided into two subgroups: a) no 6TG group and b) 6TG group. The cell culture medium with siRNA was removed from each flask and fresh medium containing 100 μM 6TG was added into each flask of 6TG groups and fresh medium without 6TG was added to each flask in no 6TG groups. All cells were incubated for another 3 days then all cells were split into fresh medium without 6TG. After a final 3-day incubation, all cells were harvested. The genomic DNA samples were extracted from all samples collected using Gentra Puregene Cell Kit (QIAGEN INC, Cat #158767) following manufacturer's instructions and sent for next generation sequencing (NGS) barcode sequencing. The NGS sequencing results were analyzed by OGA algorithm (Meisen et al., Mol Ther Methods Clin Dev, Vol. 17:601-611, 2020). False discovery rate (FDR)<0.2 was used as cutoff line. All samples maintained good representation of gRNA library—roughly 77,000 gRNAs present with similar overall distribution. In addition, gRNAs that target HPRT1 were successfully enriched by about 2-fold in 6TG treated vs. no 6TG group (data not shown).

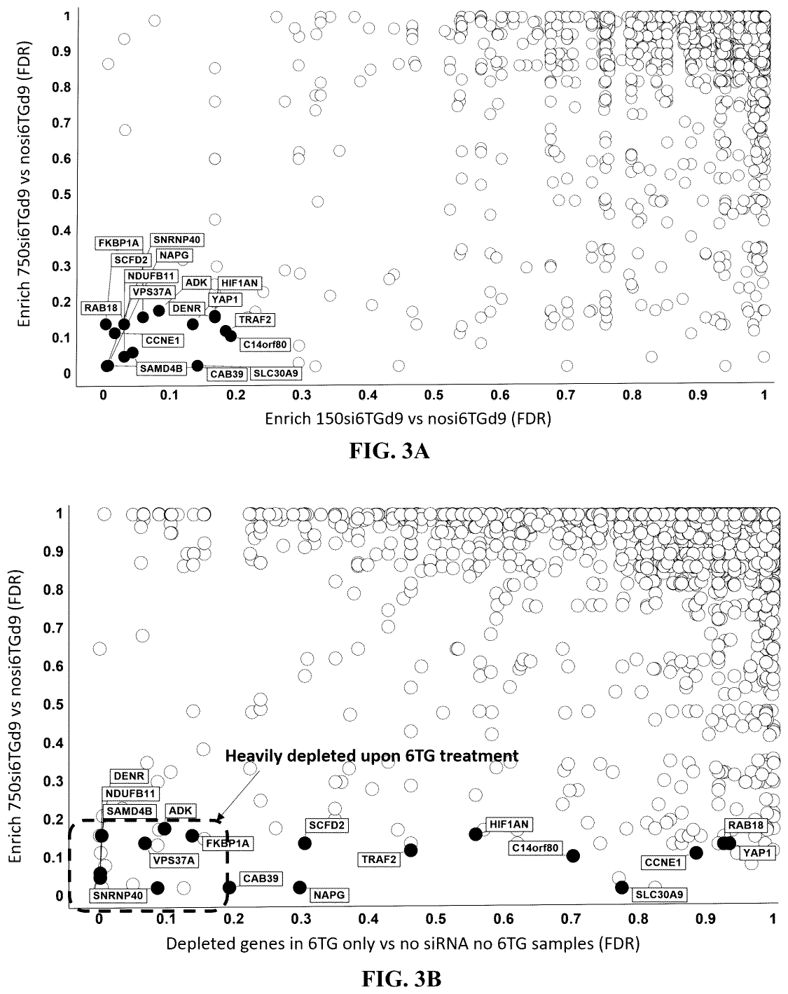

In order to identify genes that when knocked out can improve GalNAc moiety-conjugated siRNA internalization, trafficking or RNAi activity, the analysis was focused on gRNAs that were enriched in samples treated with both siRNA and 6TG but were not enriched in the 6TG only treated control group. These hits include genes that when knocked out could: 1) enhance HPRT1 siRNA silencing potency, 2) increase sensitivity to 6TG in the absence of siRNA, or 3) enhance cell viability in the presence of 6TG. To identify the genes with the most potent effects, gene hits that were significantly (FDR<0.2) enriched in both high dose (750 nM) and low dose (150 nM) GalNAc moiety-conjugated HPRT1 siRNA and 6TG treated groups, compared to the 6TG only treatment group, were selected (FIG. 3A). This analysis identified the following 17 genes: ADK, C14orf80 (also known as TEDC1), CAB39, CCNE1, DENR, FKBP1A, HIF1AN, NAPG, NDUFB11, RAB18, SAMD4B, SCFD2, SLC30A9, SNRNP40, TRAF2, VPS37A, and YAP1 (FIG. 3A). To understand whether any of these 17 genes have an impact on the sensitivity of the cells to 6TG treatment in the absence of siRNA treatment, these genes were plotted with the genes depleted in the 6TG alone treated group (no siRNA) vs. samples from cells that were not treated with siRNA or 6TG (no siRNA no 6TG samples) (FIG. 3B). In FIG. 3B, the horizontal axis indicates the sensitivity to 6TG. The genes that enhance the sensitivity to 6TG when their expression is knocked out and lead to strong cell death upon 6TG treatment are enriched on the horizontal axis with smaller FDR. When FDR<0.2 was set as the cutoff, 8 genes were identified as promoting sensitivity to 6TG treatment (FIG. 3B). The remaining 9 genes when knocked out had no impact on 6TG sensitivity as shown by the larger FDR on horizontal axis. The enrichment of these 9 genes (RAB18, YAP1, CCNE1, SLC30A9, C14orf80 (also known as TEDC1), HIF1AN, TRAF2, NAPG and SCFD2) was most likely to be directly related to a role in siRNA delivery and activity. Accordingly, inhibition of expression of these genes or the activity of the proteins encoded by these genes may enhance silencing activity of ligand-conjugated oligonucleotide compounds, such as GalNAc moiety-conjugated siRNA molecules.

### Example 2. Validation of Protein Regulators of siRNA Silencing Activity

Independent validation of candidates identified using the HPRT1-6TG selection method with a different assay system is necessary. To validate the hits identified in the genome wide loss of function screen described in Example 1, a secondary screen using a multiplexed synthetic gRNA system (Synthego Corporation, Redwood City, CA), was used. In this multi-guide strategy, three gRNAs designed in close proximity to one another are delivered together to Cas9+ cells to induce a large deletion in the target gene and more efficient target gene knockout than a single gRNA.

The multiplexed synthetic gRNAs for 58 selected genes, including genes identified in the initial screen described in Example 1 (RAB18, CCNE1, SLC30A9, NAPG, SCFD2, VPS37A, SAMD4B and CAB39) along with some control genes (AGO2, ASGR1, and ASGR2) were transfected into Hep3BCas9 stable cells in a 96-well plate format using Lipofectamine CRISPRMAX Cas9 Transfection Reagent (Invitrogen, Cat #CMAX00008). 1.5 μL of 0.3 μM multiplexed synthesized gRNA was first mixed with 8.5 μL Opti-MEM medium in each well. 0.2 μL of CRISPRMAX reagent diluted in 5 μL of Opti-MEM medium was then added to each well and incubated at room temperature for 5 to 10 minutes. After incubation, 85 μL (15,000 cells per well) of Hep3BCas9 stable cells were added to each well. The plate was allowed to sit for 20 minutes prior to placing it in 37° C. tissue culture incubator, and transfection medium was replaced with EMEM containing 10% FBS and 1% AA (Antibiotic Antimycotic Solution) at ˜6 hours after transfection. The cells were split at 1:6 ratio on day 3 post-incubation. The cells were incubated for a total of 6 days after CRISPRMAX transfection to allow protein knockdown. On day 6 post-transfection, the cells were treated with GalNAc moiety conjugated-HPRT1 siRNA (duplex no. 8172), HPRT1 siRNA conjugated to an anti-ASGR1 antibody (duplex no. 6709), or HPRT1 siRNA conjugated to cholesterol (duplex no. 17102). The structure of these HPRT1 siRNA conjugates is described in Table 5 below. HPRT1 siRNA conjugates were added to each well at the desired concentrations (500 nM, 100 nM and 20 nM) followed by 4-day incubation period in 37° C. tissue culture incubator. The total RNA of each sample was extracted by using KingFisher Flex System (Thermo Fisher Scientific) and MagMAX mirVana Total RNA Isolation Kit (Applied Biosystems, Cat #A27828) as per manufacturer instructions. The cDNA was then synthesized from total RNA sample using the Applied Biosystems High Capacity Reverse Transcription Kit (Cat #4368813) and used to quantify siRNA activity by ddPCR (Droplet Digital Polymerase Chain Reaction). ddPCR reactions were assembled using BioRad's ddPCR Supermix for Probes (Cat #1863010) as per the user manual. Droplets were then generated by QX200 Automated Droplet Generator (BioRad, Cat #1864101). Thermal cycling reactions were then performed on C1000 Touch Thermal Cycler with 96-Deep Well Reaction Module (BioRad, Cat #1851197) (BioRad, Cat #1851197). The reactions were then read by QX200 Droplet Reader (BioRad, Cat #1864003) and analyzed by using BioRad's QuantaSoft software package. The predesigned primer/probe for ddPCR assays were obtained from Integrated DNA Technologies (Coralville, IA) with 3.6:1 primer to probe ratio. The assay ID of primer/probe used for quantifying HPRT1 gene and the housekeeping TBP (TATA-Box Binding Protein) gene were Hs.PT.39a.22214821 and Hs.PT.58.19489510, respectively. The ddPCR copy number readings (copies/20 μL) of both target gene (HPRT1) and housekeeping TBP gene were recorded for each well. The normalized target gene mRNA level was calculated by dividing the ddPCR reading of the target gene by the ddPCR reading of TBP taken from the same well. The resulting number of siRNA treated sample was further divided by the number of no siRNA treatment sample to obtain the percentage reading of the target gene mRNA level.

HPRT1 siRNA silencing efficacy as measured by ddPCR (normalized to no siRNA control) in cells with select genes knocked out is shown in Table 3 below. As expected, when AGO2 is knocked out by multiplexed synthetic gRNA, the HPRT1 siRNA silencing activity is abolished with all tested siRNA conjugates. Because ASGR1 is a critical component of ASGPR, ASGR1 CRISPR-KO leads to loss of response to GalNAc moiety conjugated-HPRT1 siRNA as well as to anti-ASGR1 antibody conjugated HPRT1 siRNA. However, knocking out ASGR1 had no impact on the function of cholesterol conjugated HPRT1 siRNA. These results indicate that the multiplexed synthetic gRNA system was working as expected. As shown in Table 3, the CRISPR screen hits RAB18, SCFD2, NAPG, and SAMD4B when knocked out by multiplexed synthetic gRNA enhanced the efficacy of the siRNA conjugates to different degrees. VPS37A specifically enhanced the efficacy of cholesterol-conjugated siRNA. Other screen hits, CAB39, CCNE1 and SLC30A9, could not be validated by the multiplexed synthetic gRNA approach. Proteins encoded by ZW10 and STX18 have been shown to interact with RAB18 protein (Xu et al., J Cell Biol, Vol. 217:975-995, 2018; Li et al., Cell Rep, Vol. 27:343-358 e345, 2019). Knocking out ZW10 and STX18 by multiplexed synthetic gRNA also enhanced siRNA silencing efficacy (Table 3). The results of the experiments in this Example show that several of the genes identified as potential regulators of siRNA silencing activity, including RAB18, SCFD2, NAPG, VPS37A, and SAMD4B, were validated through a secondary arrayed CRISPR screen system by using multiplexed synthetic gRNA.

TABLE 3
Impact of select gene knock outs on HPRT1 mRNA levels
in Hep3B cells treated with different siRNA conjugates¹

| | Treatment | | | | |
|---|---|---|---|---|---|
| | | | | | 6709 |
| | 8172 | 8172 | 8172 | 17102 | (ASGR1 |
| | (GalNAc | (GalNAc | (GalNAc | (Cholesterol | antibody |
| Gene | conjugate) | conjugate) | conjugate) | conjugate) | conjugate) |
| Knockout | 500 nM | 100 nM | 20 nM | 100 nM | 100 nM |
| SCFD2 | 13.7 | 21.0 | 41.2 | 23.9 | 21.9 |
| ZW10 | 15.7 | 26.1 | 45.9 | 27.6 | 31.5 |
| RAB18 | 19.4 | 28.3 | 50.9 | 32.2 | 31.0 |
| STX18 | 24.9 | 43.2 | 70.6 | 25.9 | 45.6 |
| NAPG | 24.9 | 33.8 | 70.3 | 30.0 | 28.4 |
| VPS37A | 25.8 | 52.0 | 89.9 | 18.7 | 62.3 |
| SAMD4B | 27.9 | 44.4 | 69.4 | 34.9 | 47.6 |
| Hep3BCas9_No | 28.5 | 48.5 | 84.9 | 41.7 | 48.9 |
| gRNA | | | | | |
| CAB39 | 30.8 | 50.1 | 82.4 | 42.2 | 51.8 |
| SLC30A9 | 36.1 | 48.2 | 80.0 | 39.9 | 59.5 |
| CCNE1 | 48.9 | 57.2 | 95.0 | 39.0 | 50.6 |
| ASGR1 | 72.5 | 109.1 | 114.4 | 46.8 | 96.3 |
| ASGR2 | 83.3 | 85.6 | 140.2 | 40.2 | 78.6 |
| AGO2 | 93.5 | 108.0 | 132.3 | 104.3 | 91.2 |
| No gRNA_No | 100.0 | 100.0 | 100.0 | 100.0 | 100.0 |
| siRNA | | | | | |

¹HPRT1 mRNA levels are presented as percentage of HPRT1/TBP mRNA signals detected through ddPCR and normalized to no siRNA control

### Example 3. Inhibition of RAB18 Expression Enhances the Silencing Effect of Multiple siRNA Conjugates

Because RAB18 was the only RAB family member detected in the loss-of-function screen, and because the RAB family is important in regulating intracellular vesicle trafficking, further experiments were performed to understand the mechanisms by which RAB18 regulates siRNA activity in Hep3B cells. To study the function of RAB18, three different siRNA molecules targeting the human RAB18 gene (siRAB18-1, siRAB18-2, and siRAB18-3) were obtained from Ambion (Austin, TX; catalog #4390824, siRNA ID nos. s22703, s22704, and s22705) and were validated for their silencing potency of RAB18 in Hep3B cells. The nucleobase sequences of the sense and antisense strands for each of the RAB18-targeted siRNA molecules is provided below in Table 4. Each siRNA molecule had a 19 base pair duplex region with a 2-nucleotide overhang at both the 3′ end of the sense strand and the 3′ end of the antisense strand. A non-targeting siRNA (siNTC, Invitrogen; catalog #4390843) was used as a negative control.

TABLE 4
Sequences for RAB18-targeted siRNA molecules

| | | SEQ | | SEQ |
|---|---|---|---|---|
| Molecule | Sense Sequence | ID | Antisense Sequence | ID |
| ID | (5′-3′) | NO: | (5′-3′) | NO: |
| siRAB18-1 | GGAUGGAAAUAAGGCUAAATT | 5 | UUUAGCCUUAUUUCCAUCCAC | 8 |
| (s22703) | | | | |
| siRAB18-2 | GGUUCACAGAUGAUACGUUTT | 6 | AACGUAUCAUCUGUGAACCTC | 9 |
| (s22704) | | | | |
| siRAB18-3 | GGAAAAUCGUGAAGUCGAUTT | 7 | AUCGACUUCACGAUUUUCCTT | 10 |
| (s22705) | | | | |

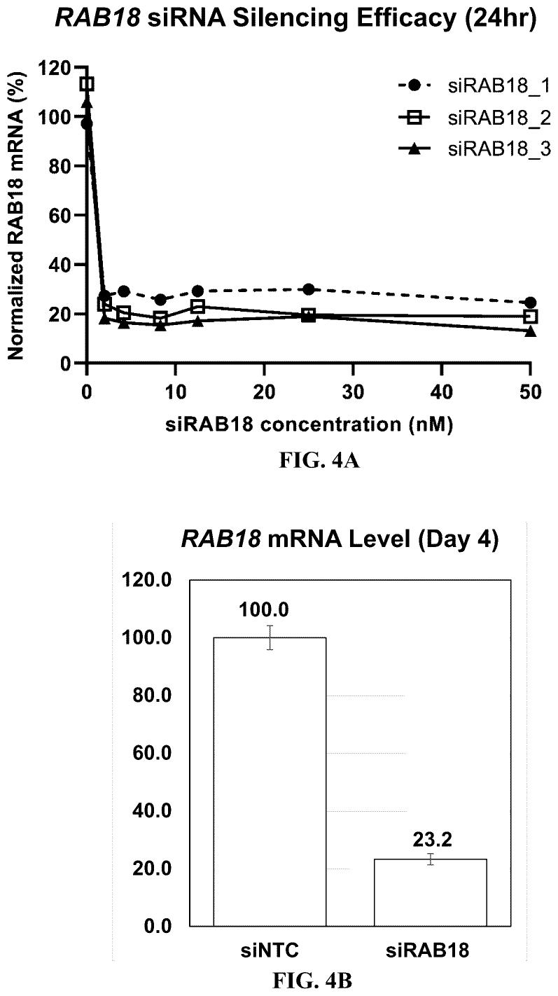

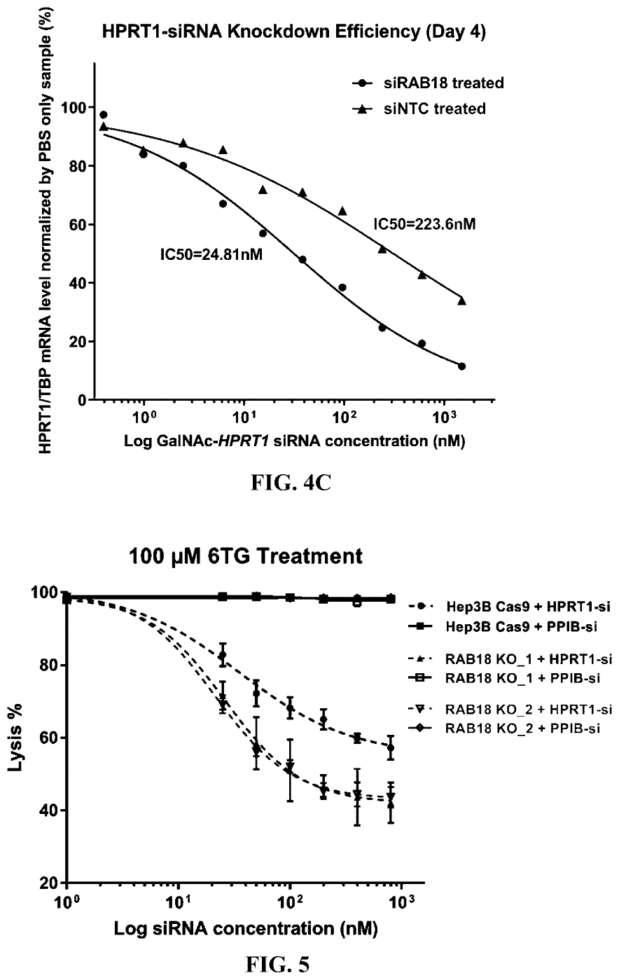

To test the efficacy of the RAB18-targeted siRNA molecules, several concentrations of each of three siRNA molecules (0.24 nM to 50 nM) or sterile water (negative control) was individually reverse transfected in duplicate into Hep3B cells using lipofectamine RNAiMAX (Invitrogen, cat #13778075). 24 hours post-transfection, cells were lysed and harvested for RNA using MagMAX mirVana Total RNA Isolation kit (Applied Biosystems, Cat #A27828) and reverse transcribed for ddPCR analysis using the Applied Biosystems High Capacity Reverse Transcription Kit (Cat #4368813), according to manufacturer instructions. The RAB18 ddPCR readings normalized by housekeeping gene TBP were used to calculate a normalized percentage of RAB18 mRNA level. Results are shown in FIG. 4A. Among the three tested RAB18-targeted siRNA molecules, siRAB18-3 exhibited the highest potency in reducing expression of RAB18 (FIG. 4A) and thus was selected for further experiments to study the function of RAB18 on the silencing activity of a GalNAc moiety-conjugated siRNA molecule targeting HPRT1.

For analysis of the effect of RAB18 knockdown on GalNAc moiety-conjugated HPRT1 siRNA efficacy, a non-targeting control siRNA molecule (siNTC) (50 nM) or siRAB18-3 (50 nM) was reverse transfected into Hep3B cells. 24 hours post-transfection, cells were trypsinized and washed twice in EMEM to remove residual transfection reagent, then plated into 96-well plates containing either PBS or multiple concentrations of a GalNAc moiety-conjugated HPRT1 siRNA (duplex no. 8172). On day 4 post GalNAc-HPRT1 siRNA conjugate treatment, the cells were lysed for RNA isolation and cDNA synthesis as described above. As illustrated in FIG. 4B, RAB18 mRNA measured by ddPCR was maintained at a low level (23.2%) in cells transfected with siRNA18-3 on day 4 post treatment with duplex no. 8172 as compared to RAB18 mRNA levels in cells transfected with siNTC. The level of HPRT1 mRNA was also measured by ddPCR on day 4 post treatment with duplex no. 8172. As shown in FIG. 4C, the efficacy of the GalNAc moiety-conjugated HPRT1 siRNA in reducing HPRT1 expression was greater in Hep3B cells transfected with siRAB18-3 as compared to that in cells transfected with siNTC. In particular, the IC50 for the GalNAc-HPRT1 siRNA conjugate in siRAB18-3 transfected cells was 24.8 nM versus 223.6 nM in siNTC transfected cells (FIG. 4C), which was a 10-fold improvement in potency.

Next, to completely abolish the function of RAB18, two RAB18 knockout pools (RAB18_KO_1 and RAB18_KO_2) were created by transducing two lentiviral gRNA vectors targeting RAB18 (SIGMA vector: U6-gRNA: PGK-puro-2A-tagBFP) into Hep3BCas9 cells. The structural features for the RAB18 gRNA lentivirus vectors were:
RAB18 gRNA lentiviral vector #1: Sanger clone ID: HS5000033611; DNA target sequence: TAACTCCCAGCTATTATAGAGG (SEQ ID NO: 11)
RAB18 gRNA lentiviral vector #2: Sanger clone ID: HS5000033612; DNA target sequence: GCTATTATAGAGGTGCACAGGG (SEQ ID NO: 12)

The RAB18 knockout efficiency was verified by Amplicon-EZ sequencing (data not shown). Knocking out the RAB18 gene did not alter cell viability in Hep3BCas9 cells (data not shown). Because RAB18 was identified through HPRT1-6TG selection screen, the same HPRT1-6TG screening assay was repeated in RAB18 knockout cells. Specifically, Hep3BCas9 cells and the two RAB18 knockout cells were treated with various concentrations of either a GalNAc moiety-conjugated HPRT1 siRNA (duplex no. 8172) or a GalNAc moiety-conjugated PPIB (peptidyl-prolyl cis-trans isomerase B) siRNA (duplex no. 8714; see Table 5 for sequences) on day 0 of the experiment. On day 3 of the experiment, the tissue culture media with the siRNA conjugates was removed and then 100 μM 6TG was added to the cells. The cells were incubated for 4 days after 6TG treatment. Then cells were split, the media removed, and fresh 6TG added on day 7 and cultured for additional 2 days. Cell lysis rate was measured on day 6 post-6TG treatment using CellTiter-Glo reagents (Promega, Madison, WI). As shown in FIG. 5, compared with the parental Hep3BCas9 cells, when treated with the GalNAc moiety-conjugated HPRT1 siRNA approximately 15% more RAB18 knockout cells were able to survive under 6TG selection (58% in RAB18 knockout cells compared to 43% in Hep3BCas9 cells at the highest siRNA dose tested), indicating that HPRT1 siRNA induced greater gene silencing in RAB18 knockout cells than in Hep3BCas9 parental cells. Neither Hep3BCas9 cells nor RAB18 knockout cells treated with GalNAc moiety-conjugated siRNA targeting PPIB gene as a non-relevant siRNA control showed enhanced resistance to 6TG treatment (FIG. 5).

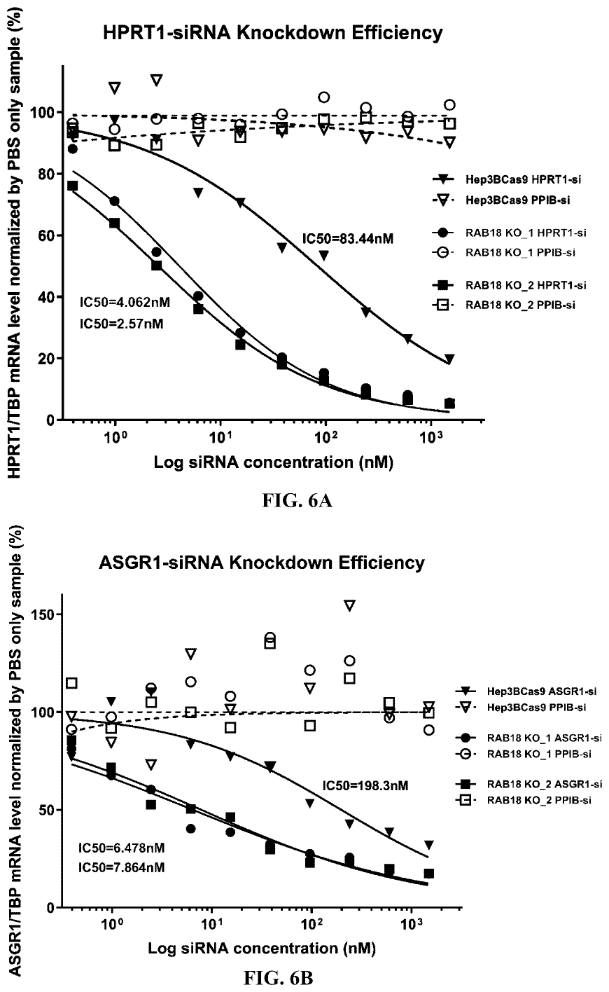

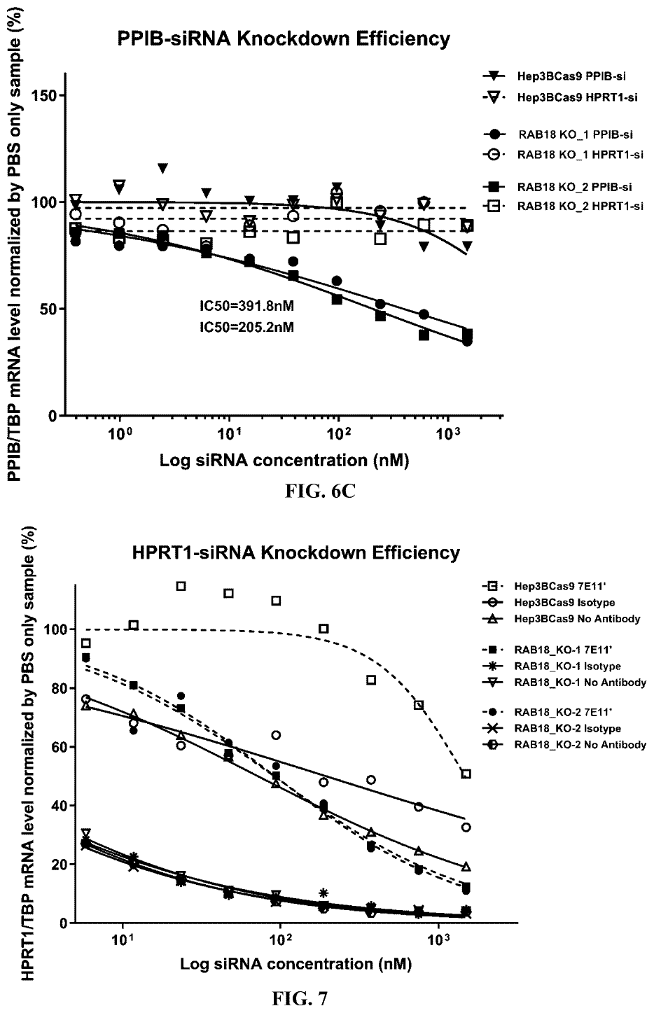

Next, the effect of knocking out RAB18 on the silencing efficacy of three different GalNAc moiety-conjugated siRNA molecules was evaluated. Hep3BCas9 cells and RAB18 knockout cells were treated with three GalNAc moiety-conjugated siRNAs: HPRT1 siRNA (duplex no. 8172), ASGR1 siRNA (duplex no. 16084), and PPIB siRNA (duplex no. 8714) for four days. Sequences for each of the siRNA molecules are described in Table 5 below. The cells were lysed and RNA was extracted and reverse transcribed for ddPCR analysis as described above. The assay ID of primer/probe used for quantifying HPRT1 gene and the housekeeping TBP gene were the same as those described in Example 2. The assay ID of primer/probe used for quantifying ASGR1 gene and the PPIB gene were Hs.PT.56a.24725395 and Hs.PT.58.40006718, respectively. The ddPCR readings for each target gene (HPRT1, ASGR1, or PPIB) were normalized by the ddPCR readings for housekeeping TBP gene and expressed as a percentage of the corresponding mRNA levels in the PBS (phosphate buffered saline)-treated control cells (i.e. cells that were not treated with the GalNAc-siRNA conjugate molecules) For all three tested GalNAc-siRNA conjugates, the target gene knockdown was greater in RAB18 knockout cells compared to Hep3BCas9 parental cells (FIGS. 6A, 6B, and 6C). The IC50 for the GalNAc-HPRT1 siRNA conjugate in Hep3BCas9 was 83.4 nM compared to 2.6 nM and 4.1 nM in the two RAB18 knockout lines, a 20˜30-fold change (FIG. 6A). A similar increase in siRNA silencing potency was observed for the GalNAc-ASGR1 siRNA conjugate in the RAB18 knockout cell lines. For the GalNAc-ASGR1 siRNA conjugate, the IC50 was 198.3 nM in Hep3BCas9 cells and 7.9 nM or 6.5 nM in the two RAB18 knockout cells (FIG. 6B). Compared to HPRT1 and ASGR1, PPIB is a highly expressed gene in Hep3B cells that could not be effectively silenced by the GalNAc moiety-conjugated PPIB siRNA in Hep3BCas9 cells (FIG. 6C). However, the same GalNAc-PPIB siRNA conjugate was able to silence PPIB expression in the two RAB18 knockout pools (IC50=205.2 nM or 391.8 nM) (FIG. 6C), indicating that suppression of RAB18 can enhance the silencing potency of GalNAc-siRNA conjugate molecules. The siRNA silencing efficacy for all three GalNAc-siRNA conjugate molecules was also evaluated at day 11. In this set of experiments, cells were treated with the GalNAc-siRNA conjugate molecules for 4 days and then maintained in media without the GalNAc-siRNA conjugate molecules for another 7 days, at which time the cells were lysed and RNA was extracted and reverse transcribed for ddPCR analysis. Although the silencing effect declined as the cells proliferated over time, the silencing potency was greater in RAB18 knockout cells than in Hep3BCas9 cells. For example, when treated with GalNAc-HPRT1 siRNA conjugate, the IC50 at day 11 was 363.6 nM in Hep3BCas9 cells compared to 41.3 nM and 58.3 nM in the two RAB18 knockout pools. These results demonstrate that inhibition of RAB18 expression enhances the silencing potency of GalNAc moiety-conjugated siRNA molecules regardless of the gene targeted by the siRNA molecule.

To further explore the mechanism by which RAB18 may regulate efficacy of ligand-conjugated siRNA molecules, it was first tested whether ASGR1 was required for GalNAc-siRNA conjugates to function in RAB18 knockout cells using an antibody blocking test. The Hep3BCas9 cells and RAB18 knockout cells were first pre-incubated with an anti-ASGR1 antibody (clone #7E11 described in WO 2017/058944), isotype control antibody, or no antibody for half an hour, followed by addition of the GalNAc moiety-conjugated HPRT1 siRNA (duplex no. 8172) at different concentrations. The final antibody concentration was 50 μg/mL and 2,000 cells were seeded in each well. After incubating in 37° C. tissue culture incubator for 4 days, the cells were lysed and RNA was extracted, reverse transcribed and subject to ddPCR analysis as described above. Pre-treatment of the cells with the 7E11 anti-ASGR1 antibody reduced the efficacy of the GalNAc-HPRT1 siRNA conjugate in silencing the HPRT1 gene in Hep3BCas9 and RAB18 knockout cells (FIG. 7). Similar results were obtained when the same experiment was performed using a GalNAc-ASGR1 siRNA conjugate and a GalNAc-PPIB siRNA conjugate to silence ASGR1 and PPIB genes, respectively (data not shown). The results of this set of experiments show that ASGR1 is required for delivery of GalNAc-siRNA conjugates into Hep3B cells and RAB18 knockout cells.

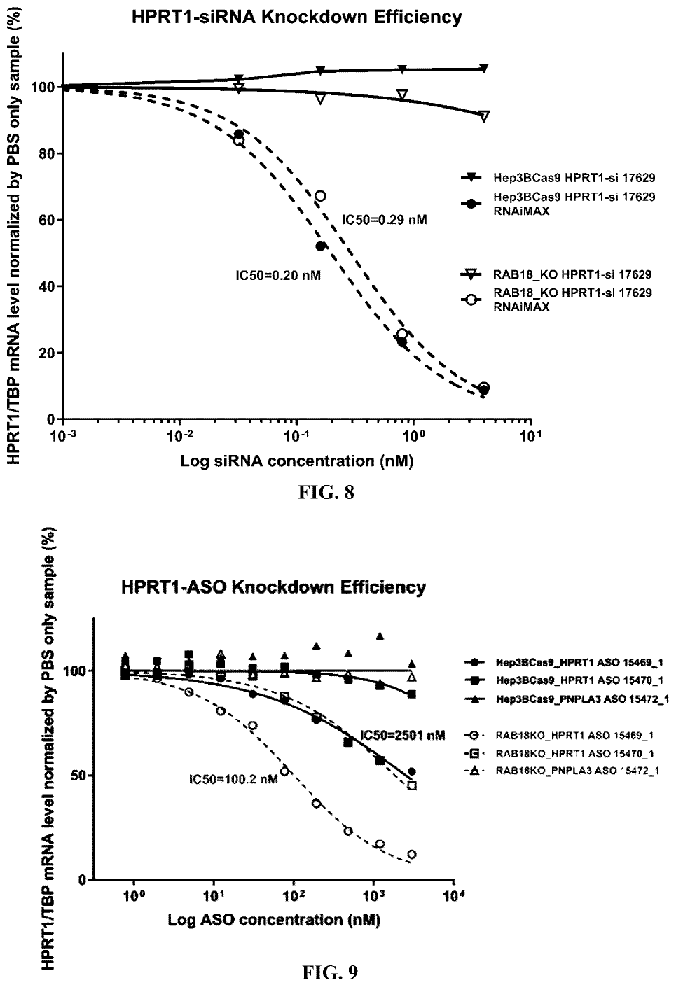

After confirming that knocking out RAB18 enhances siRNA potency of GalNAc moiety-conjugated siRNA molecules delivered through the ASGPR, we asked if knocking out RAB18 could enhance siRNA potency of siRNA molecules delivered through lipofectamine-mediated transfection. To address this question, Hep3BCas9 and RAB18 knockout cells were treated with various concentrations of an unconjugated HPRT1 siRNA (duplex no. 17629; sequences for which are set forth in Table 5) with or without lipofectamine RNAiMAX reagent (Invitrogen, Waltham, MA). As shown in FIG. 8, when the HPRT1 siRNA molecule was delivered to cells using lipofectamine-mediated transfection, the potency of the siRNA molecule in reducing HPRT1 expression was similar between Hep3BCas9 cells (IC50=0.2 nM) and RAB18 knockout cells (IC50=0.3 nM). This finding indicates that suppression of RAB18 activity does not enhance activity of siRNA molecules delivered via lipofectamine-mediated transfection.

Taken together, the results of the experiments described in this example demonstrate that inhibiting RAB18 expression significantly enhances by at least 20-fold the silencing potency of siRNA molecules delivered to cells via a cell-surface receptor, like ASGPR. RAB18 has been implicated in various physiological processes, including regulation of lipid droplet (LD) formation (Xu et al., J Cell Biol, Vol. 217:975-995, 2018; Martin et al., J Biol Chem, Vol. 280:42325-42335, 2005), inhibition of COPI independent retrograde trafficking from Golgi to endoplasmic reticulum (ER) (Dejgaard et al., J Cell Sci, Vol. 121:2768-2781, 2008), regulation of secretory granules and peroxisomes (Vazquez-Martinez et al., Traffic, Vol. 8:867-882, 2007; Gronemeyer et al., FEBS Lett, Vol. 587:328-338, 2013), promotion of hepatitis C virus (HCV) assembly on the LD membrane (Salloum et al., PLOS Pathog, Vol. 9, e1003513, 2013), and regulation of normal ER structure (Gerondopoulos et al., J Cell Biol, Vol. 205:707-720, 2014). Although the mechanism by which RAB18 may regulate silencing activity of oligonucleotide compounds is not year clear, it may be related to RAB18's function in regulation of ER-LD tethering. As described in Example 2 and shown in Table 3 above, knocking out the genes ZW10 and STX18 (encoding Syntaxin18) by multiplexed synthetic gRNA enhanced siRNA silencing efficacy, indicating that genes interacting with RAB18 to regulate ER-LD tethering have the same suppressive effect on siRNA silencing activity. The ER has been reported to be a central nucleation site of siRNA-mediated silencing and an ER membrane resident protein (CLIMP-63) has been proven to interact with and stabilize Dicer (Stalder et al., EMBO J, Vol. 32:1115-1127, 2013, Pepin et al., Nucleic Acids Res, Vol. 40:11603-11617, 2012). Suppressing RAB18 function may enhance retrograde transport of endosomes, which would contain siRNA or other oligonucleotide compounds internalized by receptor-mediated endocytosis, to the ER, a potential subcellular silencing site for siRNA molecules. RAB18 is a universally expressed gene across multiple tissue types and is highly conserved across species. Thus, suppressing RAB18 expression or activity in cells and tissues other than the liver may also enhance the potency of ligand-conjugated siRNA molecules.

### SiRNA Molecules

Table 5 below lists the sense and antisense sequences and the type of ligand conjugated to the siRNA molecule for each of the ligand-conjugated siRNA molecules employed in the experiments described in Examples 1-3. The nucleotide sequences in Table 5 are listed according to the following notations: a, u, g, and c=corresponding 2′-O-methyl ribonucleotide; Af, Uf, Gf, and Cf=corresponding 2′-deoxy-2′-fluoro (“2′-fluoro”) ribonucleotide; and invAb=inverted abasic nucleotide (i.e. abasic nucleotide linked to adjacent nucleotide via a substituent at its 3′ position (a 3′-3′ linkage) when on the 3′ end of a strand or linked to adjacent nucleotide via a substituent at its 5′ position (a 5′-5′ internucleotide linkage) when on the 5′ end of a strand). Insertion of an “s” in the sequence indicates that the two adjacent nucleotides are connected by a phosphorothiodiester group (e.g. a phosphorothioate internucleotide linkage). Unless indicated otherwise, all other nucleotides are connected by 3′-5′ phosphodiester groups. For molecules conjugated to a GalNAc ligand, a GalNAc moiety having a structure as shown in Formula VII (supra) was conjugated to the 5′ end of the sense strand of the indicated siRNA molecule via a phosphorothioate linkage. Duplex No. 17102 was conjugated to cholesterol through the 5′ end of the sense strand, whereas duplex no. 6709 was conjugated to the anti-ASGR1 antibody through the 3′ end of the sense strand.

TABLE 5
Structure of exemplary siRNA-ligand conjugates

| | | | | SEQ | | SEQ |
|---|---|---|---|---|---|---|
| Duplex | Gene | Type of | | ID | | ID |
| No. | Target | ligand | Sense Sequence (5′-3′) | NO | Antisense Sequence (5′-3′) | NO |
| 8172 | HPRT1 | GalINAc | uccuaugaCfuGfUfAfGfauuuuas[invAb] | 13 | asUfsaaaaUfcuacAfgUfcauaggasusu | 19 |
| 6709 | HPRT1 | Anti- | UfcCfuAfuGfaCfuGfuAfgAfuUfuUfaUf | 14 | aUfaAfaAfuCfuAfcAfgUfcAfuAfgGfa | 20 |
| | | ASGR1 | | | sAfsu | |
| | | Antibody | | | | |
| 17102 | HPRT1 | Cholesterol | uscscuaugaCfuGfUfAfGfauuuuas[invAb] | 15 | asUfsaaaaUfcuacAfgUfcauaggasusu | 21 |
| 17629 | HPRT1 | None | usccuaugaCfuGfUfAfGfauuuuas[invAb] | 16 | asUfsaaaaUfcuacAfgUfcauaggasusu | 22 |
| 8714 | PPIB | GaINAc | uuggaaagAfcUfGfUfUfccaaaas[invAb] | 17 | usUfsuuugGfaacaGfuCfuuuccaasusu | 23 |
| 16084 | ASGR1 | GaINAc | gggaagaaAfgAfUfGfAfagucgcs[invAb] | 18 | asGfscgacUfucauCfuUfucuucccsusu | 24 |

### Example 4. Inhibition of RAB18 Expression Enhances the Silencing Effect of GalNAc-Conjugated Antisense Oligonucleotides

Single-stranded antisense oligonucleotides (ASO) are another type of oligonucleotide compound that is widely used to silence gene expression. To understand if RAB18 also regulates the efficacy of ASO-mediated gene silencing, the impact of knocking out RAB18 on ASO silencing efficacy was studied. Hep3BCas9 parental cells and RAB18 knockout cells described in Example 3 were treated for four days with either PBS or multiple concentrations of one of two GalNAc moiety conjugated ASO molecules targeting the HPRT1 gene (compound nos. 15469 and 15470) or a control GalNAc moiety conjugated ASO molecule targeting the PNPLA3 gene (compound no. 15472). Sequences for each of the three GalNAc moiety conjugated ASO molecules are described in Table 6 below. Cell suspension at 4E+05 cells/ml concentration was made in EMEM+10% FBS medium for Hep3BCas9 parental cells and RAB18 knockout cells. The cell solution was plated in a 96-well plate at the amount of 50 μl/well. Immediately after plating the cells, 50 μl of GalNAc-ASO conjugate diluted in EMEM+10% FBS medium at various concentrations was added to each well. The 96-well plate was then incubated at 37° C. in a tissue culture incubator for 4 days. On day 4 post-treatment with the GalNAc moiety conjugated ASO molecules, the cells were lysed and the RNA sample from each well was extracted and reverse transcribed into cDNA. The silencing efficacy of each of the different GalNAc moiety conjugated ASO molecules on HPRT1 mRNA expression was then measured by ddPCR analysis. The ddPCR readings for the HPRT1 gene were normalized by the ddPCR readings for housekeeping TBP gene and expressed as a percentage of the corresponding mRNA levels in the PBS-treated control cells (i.e. cells that were not treated with the GalNAc-ASO conjugate molecules).

As demonstrated in FIG. 9, treatment with the control GalNAc-conjugated ASO molecule targeting PNPLA3 gene (compound no. 15472) had no effect on HPRT1 expression level in either the Hep3BCas9 cells or the RAB18 knockout cells. Enhanced silencing effects on HPRT1 expression were observed in RAB18 knockout cells when compared with Hep3BCas9 parental cells in the groups treated with GalNAc-conjugated ASO molecules targeting HPRT1 (FIG. 9). For example, the IC50 value for compound no. 15469 in Hep3BCas9 parental cells was 2501 nM, whereas the IC50 value for the same compound in RAB18 knockout cells was 100 nM, a 25-fold enhancement of silencing efficacy. These results show that RAB18 regulates an intracellular step similarly used by both GalNAc-conjugated siRNA molecules and GalNAc-conjugated ASO molecules. Therefore, inhibition of RAB18 expression is a viable approach to enhancing the silencing activity of ligand-conjugated ASO molecules as well as ligand-conjugated siRNA molecules.

### ASO Molecules

The nucleotide sequences for the single-stranded antisense oligonucleotide (ASO) compounds in Table 6 are listed according to the following notations: dA, dT, dG, and dC=corresponding deoxyribonucleotide; A, T, G, and C (underlined and bolded)=corresponding β-D-Methyleneoxy (4′-CH₂—O-2′) nucleotide (“locked nucleic acid” or LNA); and C*=nucleotide with 5-methylcytosine base. C*=LNA with 5-methylcytosine base. Insertion of an “s” in the sequence indicates that the two adjacent nucleotides are connected by a phosphorothiodiester group (e.g. a phosphorothioate internucleotide linkage). Unless indicated otherwise, all other nucleotides are connected by 3′-5′ phosphodiester groups. All three single-stranded ASO molecules were conjugated at the 5′ end via a phosphorothioate linkage to a GalNAc moiety having a structure as shown in Formula VII (supra).

TABLE 6
Structure of GalNAc-conjugated antisense oligonucleotides

| | | Unmodified | SEQ | | SEQ |
|---|---|---|---|---|---|
| Compound | Gene | Nucleotide Sequence | ID | Modified Nucleotide Sequence | ID |
| No. | Target | (5′-3′) | NO: | (5′-3′) | NO: |
| 15469 | HPRT1 | AAAATCTACAGTCATAGGA | 30 | dAdAdAAsTsC*sdTsdAsdCsdAsdGsdTsdCsdAsdTsdAsGsGsA | 33 |
| 15470 | HPRT1 | ATAAAATCTACAGTCATAG | 31 | dAdTdAAsAsAsdTsdCsdTsdAsdCsdAsdGsdTsdCsdAsTsAsG | 34 |
| 15472 | PNPLA3 | GGGCATATCTGGAAGCCAT | 32 | dGdGdGC*sAsTsdAsdTsdCsdTsdGsdGsdAsdAsdGsdCsC*sAsT | 35 |

All publications, patents, and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the appended claims.

Those skilled in the art will recognize, or be able to ascertain using no more than routine experimentation, many equivalents to the specific embodiments of the invention described herein. Such equivalents are intended to be encompassed by the following claims.

What is claimed:

1. A method for enhancing the silencing activity of a first oligonucleotide compound in a cell comprising:
inhibiting the expression or activity of a suppressor protein in the cell, wherein the suppressor protein is RAB18, ZW10, STX18, SCFD2, NAPG, SAMD4B, or VPS37A; and
contacting the cell with the first oligonucleotide compound, said oligonucleotide compound comprising a sequence that is substantially complementary to a sequence of a target gene, wherein the oligonucleotide compound is covalently attached to a ligand of a receptor expressed on the surface of the cell.

2. The method of claim 1, wherein the suppressor protein is RAB18, ZW10 or STX18.

3. The method of claim 1, wherein the suppressor protein is RAB18.

4. The method of any one of claims 1 to 3, wherein inhibiting the expression or activity of a suppressor protein comprises contacting the cell with a second oligonucleotide compound comprising a sequence that is substantially complementary to an mRNA sequence encoding the suppressor protein.

5. The method of claim 4, wherein the second oligonucleotide compound is single-stranded.

6. The method of claim 4, wherein the second oligonucleotide compound is double-stranded.

7. The method of any one of claims 4 to 6, wherein the second oligonucleotide compound comprises at least one modified nucleotide.

8. The method of claim 7, wherein the modified nucleotide is a 2′-modified nucleotide.

9. The method of claim 7, wherein the modified nucleotide is a 2′-fluoro modified nucleotide, a 2′-O-methyl modified nucleotide, a 2′-O-methoxyethyl modified nucleotide, 2′-O-alkyl modified nucleotide, a 2′-O-allyl modified nucleotide, a bicyclic nucleic acid (BNA), a deoxyribonucleotide, or combinations thereof.

10. The method of claim 7, wherein all of the nucleotides in the second oligonucleotide compound are modified nucleotides.

11. The method of any one of claims 4 to 10, wherein the second oligonucleotide compound comprises one or more phosphorothioate internucleotide linkages.

12. A method for reducing expression of a target gene in a cell comprising:
contacting the cell with an inhibitor of a suppressor protein, wherein the suppressor protein is RAB18, ZW10, STX18, SCFD2, NAPG, SAMD4B, or VPS37A; and
contacting the cell with a first oligonucleotide compound comprising a sequence that is substantially complementary to a sequence of the target gene, wherein the oligonucleotide compound is covalently attached to a ligand of a receptor expressed on the surface of the cell.

13. The method of claim 12, wherein the suppressor protein is RAB18, ZW10 or STX18.

14. The method of claim 12, wherein the suppressor protein is RAB18.

15. The method of any one of claims 12 to 14, wherein the inhibitor of the suppressor protein is a second oligonucleotide compound comprising a sequence that is substantially complementary to an mRNA sequence encoding the suppressor protein.

16. The method of claim 15, wherein the second oligonucleotide compound is single-stranded.

17. The method of claim 15, wherein the second oligonucleotide compound is double-stranded.

18. The method of any one of claims 15 to 17, wherein the second oligonucleotide compound comprises at least one modified nucleotide.

19. The method of claim 18, wherein the modified nucleotide is a 2′-modified nucleotide.

20. The method of claim 18, wherein the modified nucleotide is a 2′-fluoro modified nucleotide, a 2′-O-methyl modified nucleotide, a 2′-O-methoxyethyl modified nucleotide, 2′-O-alkyl modified nucleotide, a 2′-O-allyl modified nucleotide, a BNA, a deoxyribonucleotide, or combinations thereof.

21. The method of claim 18, wherein all of the nucleotides in the second oligonucleotide compound are modified nucleotides.

22. The method of any one of claims 15 to 21, wherein the second oligonucleotide compound comprises one or more phosphorothioate internucleotide linkages.

23. The method of any one of claims 1 to 22, wherein the target gene is a human gene.

24. The method of any one of claims 1 to 23, wherein expression of the target gene is associated with a disease or disorder.

25. The method of any one of claims 1 to 24, wherein the first oligonucleotide compound is a single-stranded antisense oligonucleotide comprising a sequence that is substantially complementary to the sequence of the target gene.

26. The method of claim 25, wherein the antisense oligonucleotide is about 15 to about 30 nucleotides in length.

27. The method of any one of claims 1 to 24, wherein the first oligonucleotide compound is an siRNA comprising a sense strand and an antisense strand, and wherein the antisense strand comprises a sequence that is substantially complementary to the sequence of the target gene.

28. The method of claim 27, wherein the sense strand comprises a sequence that is sufficiently complementary to the sequence of the antisense strand to form a duplex region of about 15 to about 30 base pairs in length.

29. The method of claim 27 or 28, wherein the sense strand and the antisense strand are each independently about 19 to about 30 nucleotides in length.

30. The method of any one of claims 27 to 29, wherein the sense strand and the antisense strand are each independently about 19 to about 23 nucleotides in length.

31. The method of any one of claims 1 to 30, wherein the first oligonucleotide compound comprises at least one modified nucleotide.

32. The method of claim 31, wherein the modified nucleotide is a 2′-modified nucleotide.

33. The method of claim 31, wherein the modified nucleotide is a 2′-fluoro modified nucleotide, a 2′-O-methyl modified nucleotide, a 2′-O-methoxyethyl modified nucleotide, 2′-O-alkyl modified nucleotide, a 2′-O-allyl modified nucleotide, a BNA, a deoxyribonucleotide, or combinations thereof.

34. The method of claim 31, wherein all of the nucleotides in the first oligonucleotide compound are modified nucleotides.

35. The method of any one of claims 1 to 30, wherein the first oligonucleotide compound comprises one or more phosphorothioate internucleotide linkages.

36. The method of any one of claims 1 to 35, wherein the ligand comprises a cholesterol moiety, a vitamin, a steroid, a bile acid, a folate moiety, a fatty acid, a carbohydrate, a glycoside, or antibody or antigen-binding fragment thereof.

37. The method of any one of claims 1 to 35, wherein the ligand comprises galactose, galactosamine, or N-acetyl-galactosamine.

38. The method of claim 37, wherein the ligand comprises a multivalent galactose moiety or multivalent N-acetyl-galactosamine moiety.

39. The method of claim 38, wherein the multivalent galactose moiety or multivalent N-acetyl-galactosamine moiety is trivalent or tetravalent.

40. The method of any one of claims 1 to 39, wherein the ligand is a ligand of a receptor expressed on the surface of a liver cell.

41. The method of claim 40, wherein the receptor is an asialoglycoprotein receptor.

42. The method of any one of claims 1 to 41, wherein the cell is in vitro.

43. The method of any one of claims 1 to 41, wherein the cell is in vivo.

44. The method of claim 43, wherein the cell is in a subject in need of reduced expression of the target gene.

45. The method of any one of claims 1 to 44, wherein the cell is a hepatocyte.

46. A method for reducing expression of a target gene in a subject comprising administering to the subject:
an inhibitor of a suppressor protein, wherein the suppressor protein is RAB18, ZW10, STX18, SCFD2, NAPG, SAMD4B, or VPS37A; and
a first oligonucleotide compound comprising a sequence that is substantially complementary to a sequence of the target gene, wherein the first oligonucleotide compound is covalently attached to a first ligand.

47. The method of claim 46, wherein the suppressor protein is RAB18, ZW10 or STX18.

48. The method of claim 46, wherein the suppressor protein is RAB18.

49. The method of any one of claims 46 to 48, wherein the inhibitor of the suppressor protein is a second oligonucleotide compound comprising a sequence that is substantially complementary to an mRNA sequence encoding the suppressor protein.

50. The method of claim 49, wherein the second oligonucleotide compound is single-stranded.

51. The method of claim 49, wherein the second oligonucleotide compound is double-stranded.

52. The method of any one of claims 49 to 51, wherein the second oligonucleotide is covalently attached to a second ligand.

53. The method of claim 52, wherein the second ligand is the same as the first ligand.

54. The method of any one of claims 46 to 53, wherein the target gene is a human gene.

55. The method of any one of claims 46 to 54, wherein expression of the target gene is associated with a disease or disorder in the subject.

56. The method of any one of claims 46 to 55, wherein the target gene is a gene expressed in the liver.

57. The method of any one of claims 46 to 56, wherein the first oligonucleotide compound is a single-stranded antisense oligonucleotide comprising a sequence that is substantially complementary to a sequence of the target gene.

58. The method of claim 57, wherein the antisense oligonucleotide is about 15 to about 30 nucleotides in length.

59. The method of any one of claims 46 to 56, wherein the first oligonucleotide compound is an siRNA comprising a sense strand and an antisense strand, and wherein the antisense strand comprises a sequence that is substantially complementary to a sequence of the target gene.

60. The method of claim 59, wherein the sense strand comprises a sequence that is sufficiently complementary to the sequence of the antisense strand to form a duplex region of about 15 to about 30 base pairs in length.

61. The method of claim 59 or 60, wherein the sense strand and the antisense strand are each independently about 19 to about 30 nucleotides in length.

62. The method of any one of claims 59 to 61, wherein the sense strand and the antisense strand are each independently about 19 to about 23 nucleotides in length.

63. The method of any one of claims 49 to 62, wherein the first oligonucleotide compound, the second oligonucleotide compound, or both the first and second oligonucleotide compounds comprise at least one modified nucleotide.

64. The method of claim 63, wherein the modified nucleotide is a 2′-modified nucleotide.

65. The method of claim 63, wherein the modified nucleotide is a 2′-fluoro modified nucleotide, a 2′-O-methyl modified nucleotide, a 2′-O-methoxyethyl modified nucleotide, 2′-O-alkyl modified nucleotide, a 2′-O-allyl modified nucleotide, a BNA, a deoxyribonucleotide, or combinations thereof.

66. The method of claim 63, wherein all of the nucleotides in the first oligonucleotide compound, the second oligonucleotide compound, or both the first and second oligonucleotide compounds are modified nucleotides.

67. The method of any one of claims 49 to 66, wherein the first oligonucleotide compound, the second oligonucleotide compound, or both the first and second oligonucleotide compounds comprise one or more phosphorothioate internucleotide linkages.

68. The method of any one of claims 46 to 67, wherein the first ligand, the second ligand, or both the first and second ligands comprise a cholesterol moiety, a vitamin, a steroid, a bile acid, a folate moiety, a fatty acid, a carbohydrate, a glycoside, or antibody or antigen-binding fragment thereof.

69. The method of any one of claims 46 to 67, wherein the first ligand, the second ligand, or both the first and second ligands comprise galactose, galactosamine, or N-acetyl-galactosamine.

70. The method of claim 69, wherein the first ligand, the second ligand, or both the first and second ligands comprise a multivalent galactose moiety or multivalent N-acetyl-galactosamine moiety.

71. The method of claim 70, wherein the multivalent galactose moiety or multivalent N-acetyl-galactosamine moiety is trivalent or tetravalent.

72. The method of any one of claims 46 to 71, wherein the first ligand, the second ligand, or both the first and second ligands are a ligand of a receptor expressed on the surface of a liver cell.

73. The method of claim 72, wherein the receptor is an asialoglycoprotein receptor.
